(19) United States
(12) Patent Application Publication
(10) Pub. No.: US 2008/0063603 A1
(43) Pub. Date: Mar. 13, 2008

(54) ULTRASOUND CONTRAST AGENTS AND METHODS OF MAKING AND USING THEM

(75) Inventors: Michel Schneider, Troinex (CH); Feng Yan, Grand-Lancy (CH); David Lazarus, Saint-Julien-en-Genevois (FR); Jean Brochot, Feigeres (FR); Jerome Puginier, Via Fiume (CH)

Correspondence Address: KRAMER LEVIN NAFTALIS & FRANKEL LLP, INTELLECTUAL PROPERTY DEPARTMENT, 1177 AVENUE OF THE AMERICAS, NEW YORK, NY, 10036, US

(73) Assignee: Bracco International B.V., Amsterdam (NL)

(21) Appl. No.: 11/851,769

(22) Filed: Sep. 7, 2007

Related U.S. Application Data
(63) Continuation-in-part of application No. 11/085,169, filed on Mar. 22, 2005.
Application No. 11/085,169 is a continuation of application No. 10/226,244, filed on Aug. 23, 2002, now Pat. No. 6,896,875.
Application No. 10/226,244 is a continuation of application No. 09/630,537, filed on Aug. 1, 2000, now Pat. No. 6,485,705.
Application No. 09/630,537 is a division of application No. 09/021,150, filed on Feb. 10, 1998, now Pat. No. 6,136,293.
Application No. 09/021,150 is a division of application No. 08/853,936, filed on May 9, 1997, now Pat. No. 6,110,443.
Application No. 08/853,936 is a division of application No. 08/456,385, filed on Jun. 1, 1995, now Pat. No. 5,658,551.
Application No. 08/456,385 is a division of application No. 08/315,347, filed on Sep. 30, 1994, now Pat. No. 5,531,980.
Application No. 08/315,347 is a division of application No. 08/128,540, filed on Sep. 29, 1993, now Pat. No. 5,380,519.
Application No. 08/128,540 is a division of application No. 07/775,989, filed on Nov. 20, 1991, now Pat. No. 5,271,928.
(63) Continuation-in-part of application No. 10/725,777, filed on Dec. 3, 2003.
Application No. 10/725,777 is a continuation of application No. 09/706,778, filed on Nov. 7, 2000, now abandoned.
Application No. 09/706,778 is a division of application No. 08/910,152, filed on Aug. 13, 1997, now Pat. No. 6,200,548.
Application No. 08/910,152 is a division of application No. 08/288,550, filed on Aug. 10, 1994, now Pat. No. 5,711,933.
Application No. 08/288,550 is a division of application No. 08/033,435, filed on Mar. 18, 1993, now abandoned.
Application No. 08/033,435 is a division of application No. 07/695,343, filed on May 3, 1991, now abandoned.
(63) Continuation-in-part of application No. 09/002,710, filed on Jan. 5, 1998.
Application No. 09/002,710 is a division of application No. 08/740,653, filed on Oct. 31, 1996, now Pat. No. 6,585,955.
Application No. 08/740,653 is a division of application No. 08/380,588, filed on Jan. 30, 1995, now Pat. No. 5,578,292.
Application No. 08/380,588 is a division of application No. 07/991,237, filed on Dec. 16, 1992, now Pat. No. 5,413,774.
(63) Continuation-in-part of application No. 10/355,052, filed on Jan. 31, 2003, now abandoned.
Application No. 10/355,052 is a division of application No. 09/736,361, filed on Dec. 15, 2000, now abandoned.
Application No. 09/736,361 is a division of application No. 09/151,651, filed on Sep. 11, 1998, now Pat. No. 6,187,288.
Application No. 09/151,651 is a division of application No. 08/883,592, filed on Jun. 26, 1997, now Pat. No. 5,908,610.
Application No. 08/883,592 is a division of application No. 08/420,677, filed on Apr. 12, 1995, now Pat. No. 5,686,060.
Application No. 08/420,677 is a division of application No. 08/134,671, filed on Oct. 12, 1993, now Pat. No. 5,445,813.
(63) Continuation-in-part of application No. 10/831,165, filed on Apr. 26, 2004.
Application No. 10/831,165 is a continuation of application No. 09/694,011, filed on Oct. 23, 2000, now abandoned.
Application No. 09/694,011 is a division of application No. 09/021,367, filed on Feb. 10, 1998, now Pat. No. 6,183,725.
Application No. 09/021,367 is a division of application No. 08/848,912, filed on May 1, 1997, now Pat. No. 5,846,518.
Application No. 08/848,912 is a division of application No. 08/637,346, filed on Apr. 25, 1996, now abandoned.
Application No. 08/637,346 is a division of application No. 08/352,108, filed on Nov. 30, 1994, now Pat. No. 5,556,610.
Application No. 10/831,165 is a continuation-in-part of application No. 10/061,299, filed on Feb. 4, 2002, now Pat. No. 6,881,397.
(63) Continuation-in-part of application No. 10/781,825, filed on Feb. 20, 2004, now abandoned.
Application No. 10/781,825 is a continuation of application No. 09/770,216, filed on Jan. 29, 2001, now abandoned.
Application No. 09/770,216 is a continuation-in-part of application No. 09/151,651, filed on Sep. 11, 1998, now Pat. No. 6,187,288.
Application No. 09/151,651 is a division of application No. 08/883,592, filed on Jun. 26, 1997, now Pat. No. 5,908,610.
Application No. 08/883,592 is a division of application No. 08/420,677, filed on Apr. 12, 1995, now Pat. No. 5,686,060.
Application No. 08/420,677 is a division of application No. 08/134,671, filed on Oct. 12, 1993, now Pat. No. 5,445,813.

(30) Foreign Application Priority Data
Apr. 2, 1990 (EP) EP 90810262.7
May 18, 1990 (EP) EP 90810367.4
Apr. 2, 1991 (EP) PCT/EP91/00620
Dec. 15, 1993 (EP) EP 93810885.9
Jan. 24, 1992 (EP) EP92810046.0
Nov. 2, 1992 (EP) EP92810837.2
Nov. 2, 1992 (EP) EP92810837
Nov. 2, 1992 (EP) 92810837.2
Dec. 15, 1993 (EP) 93810885.9
Jan. 24, 1992 (EP) 92810046.0
May 18, 1990 (EP) 90810367.4
Apr. 2, 1990 (EP) 90810262.7
Nov. 2, 1992 (EP) 92810837

(51) Int. Cl. A61K 49/00

(52) U.S. Cl. 424/9.52

(57) ABSTRACT

The invention is directed to injectable suspensions of gas-filled microvesicles, as well as methods of preparing and using the same, especially as ultrasound contrast agents.

### CROSS REFERENCE TO RELATED APPLICATIONS

This application is a continuation-in-part of U.S. Ser. No. 11/085,169, filed Mar. 22, 2005, which is a continuation of U.S. Ser. No. 10/226,244, filed Aug. 23, 2002, now U.S. Pat. No. 6,896,875, which is a continuation of U.S. Ser. No. 09/630,537, filed Aug. 1, 2000, now U.S. Pat. No. 6,485,705, which is a divisional of U.S. Ser. No. 09/021,150, filed Feb. 10, 1998, now U.S. Pat. No. 6,136,293, which is a divisional of U.S. Ser. No. 08/853,936 filed May 9, 1997, now U.S. Pat. No. 6,110,443, which is a divisional of U.S. Ser. No. 08/456,385, filed Jun. 1, 1995, now U.S. Pat. No. 5,658,551, which is a divisional of U.S. Ser. No. 08/315,347, filed Sep. 30, 1994, now U.S. Pat. No. 5,531,980, which is a divisional of U.S. Ser. No. 08/128,540, filed Sep. 29, 1993, now U.S. Pat. No. 5,380,519, which is a divisional of U.S. Ser. No. 07/775,989, filed Nov. 20, 1991, now U.S. Pat. No. 5,271,928, which is the national stage application of PCT/EP91/00620, filed Apr. 2, 1991, which claims the benefit of European Patent Application No. 90810262.7, filed Apr. 2, 1990, now abandoned.

This application is also a continuation-in-part of U.S. Ser. No. 10/725,777, filed Dec. 3, 2003, which is a continuation of U.S. Ser. No. 09/706,778, filed Nov. 7, 2000, now abandoned, which in turn is a divisional of U.S. Ser. No. 08/910,152 filed Aug. 13, 1997, now U.S. Pat. No. 6,200,548, which is a divisional of U.S. Ser. No. 08/288,550 filed Aug. 10, 1994, now U.S. Pat. No. 5,711,933, which is a divisional of U.S. Ser. No. 08/033,435 filed Mar. 18, 1993, now abandoned, which is a divisional of U.S. Ser. No. 07/695,343 filed May 3, 1991, now abandoned, which claims the benefit of European Patent Application No. 90810367.4, filed May 18, 1990, now abandoned.

This application is also a continuation-in-part of U.S. Ser. No. 09/002,710, filed Jan. 5, 1998, which is a divisional of U.S. Ser. No. 08/740,653, filed Oct. 31, 1996, now U.S. Pat. No. 6,585,955, which is a divisional of U.S. Ser. No. 08/380,588, filed Jan. 30, 1995, now U.S. Pat. No. 5,578,292 which is a divisional of U.S. Ser. No. 07/991,237, filed Dec. 16, 1992, now U.S. Pat. No. 5,413,774, which claims the benefit of EP 92810046.0, filed Jan. 24, 1992, now abandoned, is a continuation-in-part of U.S. Ser. No. 07/695,343, filed May 3, 1991, now abandoned, which claims the benefit of EP 90810367.4, filed May 18, 1990, now abandoned, and is a continuation-in-part of U.S. Ser. No. 07/775,989, filed Nov. 20, 1991, now U.S. Pat. No. 5,271,928, which claims the benefit of International Patent Application No. PCT/EP91/00620, filed Apr. 2, 1991, and European Patent Application No. 90810262.7, filed Apr. 2, 1990, now abandoned.

This application is also a continuation-in-part of U.S. Ser. No. 10/355,052, filed Jan. 31, 2003, which is a divisional of U.S. Ser. No. 09/736,361, filed Dec. 15, 2000, now abandoned, which is a divisional of U.S. Ser. No. 09/151,651, filed Sep. 11, 1998, now U.S. Pat. No. 6,187,288, which is a divisional of U.S. Ser. No. 08/883,592, filed Jun. 26, 1997, now U.S. Pat. No. 5,908,610, which is a divisional of U.S. Ser. No. 08/420,677, filed Apr. 12, 1995, now U.S. Pat. No. 5,686,060, which is a divisional of U.S. Ser. No. 08/134,671, filed Oct. 12, 1993, now U.S. Pat. No. 5,445,813, which claims the benefit of European Patent Application No. 92810837.2, filed Nov. 2, 1992, now abandoned.

This application is also a continuation-in-part of U.S. Ser. No. 10/831,165, filed Apr. 26, 2004, which is a continuation of U.S. Ser. No. 09/694,011, filed Oct. 23, 2000, now abandoned, which is a divisional of U.S. Ser. No. 09/021,367, filed Feb. 10, 1998, now U.S. Pat. No. 6,183,725, which is a divisional of U.S. Ser. No. 08/848,912, filed May 1, 1997, now U.S. Pat. No. 5,846,518, which is a divisional of U.S. Ser. No. 08/637,346, filed Apr. 25, 1996, now abandoned, which is a divisional of U.S. Ser. No. 08/352,108, filed Nov. 30, 1994, now U.S. Pat. No. 5,556,610, which claims the benefit of European Patent Application No. 93810885.9, filed Dec. 15, 1993, now abandoned and is a continuation-in-part of U.S. Ser. No. 07/991,237, filed Dec. 16, 1992, now U.S. Pat. No. 5,413,774.

This application is also a continuation-in-part of U.S. Ser. No. 10/781,825, filed Feb. 20, 2004, which is continuation of U.S. Ser. No. 09/770,216, filed Jan. 29, 2001, now abandoned, which is a continuation-in-part of U.S. Ser. No. 09/151,651, filed Sep. 11, 1998, now U.S. Pat. No. 6,187,288, which is a divisional of U.S. Ser. No. 08/883,592, filed Jun. 26, 1997, now U.S. Pat. No. 5,908,610, which is in turn a divisional of U.S. Ser. No. 08/420,677, filed Apr. 12, 1995, now U.S. Pat. No. 5,686,060, which is in turn a divisional of U.S. Ser. No. 08/134,671, filed Oct. 12, 1993, now U.S. Pat. No. 5,445,813, which in turn claims the benefit of European Patent Application No. 92810837, filed Nov. 2, 1992, now abandoned.

All of the aforementioned applications are hereby incorporated by reference herein in their entirety.

### BACKGROUND

It is well known that microbodies of air or a gas (defined here as microvesicles), e.g., microbubbles or microballoons, suspended in a liquid are exceptionally efficient ultrasound reflectors for echography. In this disclosure, the term “microbubble” specifically designates air or gas globules in suspension in a liquid which generally results from the introduction therein of air or a gas in divided form, the liquid preferably also containing surfactants or tensides to control the surface properties thereof and the stability of the bubbles. More specifically, one may consider that the internal volume of the microbubbles is limited by the gas/liquid interface, or in other words, the microbubbles are only bounded by a rather evanescent envelope involving the molecules of the liquid and surfactant loosely bound at the gas to liquid junction boundary.

The term “microcapsule” or “microballoon” designates preferably air or gas bodies with a material boundary or envelope formed of molecules other than that of the liquid of suspension, e.g., a polymer membrane wall. Both microbubbles and microballoons are useful as ultrasonic contrast agents. For instance, injecting into the blood-stream of living bodies suspensions of gas microbubbles or microballoons (in the range of 0.5 to 10 μm) in a carrier liquid will strongly reinforce ultrasonic echography imaging, thus aiding in the visualization of internal organs. Imaging of vessels and internal organs can strongly help in medical diagnosis, for instance for the detection of cardiovascular and other diseases.

The formation of suspensions of microbubbles in an injectable liquid carrier suitable for echography can follow various routes, such as by the release of a gas dissolved under pressure in this liquid, or by a chemical reaction generating gaseous products, or by admixing with the liquid soluble or insoluble solids containing air or gas trapped or adsorbed therein. For instance in DE-A-3529195 (Max-Planck Gesell.), there is disclosed a technique for generating 0.5-50 μm bubbles in which an aqueous emulsified mixture containing a water soluble polymer, an oil and mineral salts is forced back and forth, together with a small amount of air, from one syringe into another through a small opening. Here, mechanical forces are responsible for the formation of bubbles in the liquid.

M. W. Keller et al. (J. Ultrasound Med. 5 (1986), 439-8) have reported subjecting to ultrasonic cavitation under atmospheric pressure solutions containing high concentrations of solutes such as dextrose, Renografin-76, Iopamidol (an X-ray contrast agent), and the like. There the air is driven into the solution by the energy of cavitation.

Other techniques rely on the shaking of a carrier liquid in which air containing microparticles have been incorporated, said carrier liquid usually containing, as stabilizers, viscosity enhancing agents, e.g., water soluble polypeptides or carbohydrates and/or surfactants. It is effectively admitted that the stability of the microbubbles against decay or escape to the atmosphere is controlled by the viscosity and surface properties of the carrier liquid. The air or gas in the microparticles can consist of inter-particle or intra-crystalline entrapped gas, as well as surface adsorbed gas, or gas produced by reactions with the carrier liquid, usually aqueous. All this is fully described for instance in EP-A-0052575 (Ultra Med. Inc.) in which there are used aggregates of 1-50 μm particles of carbohydrates (e.g., galactose, maltose, sorbitol, gluconic acid, sucrose, glucose and the like) in aqueous solutions of glycols or polyglycols, or other water soluble polymers.

Also, in EP-A-0123235 and EP-A-0122624 (Schering, see also EP-A-0320433) use is made of air trapped in solids. For instance, EP-A-0122624 claims a liquid carrier contrast composition for ultrasonic echography containing microparticles of a solid surfactant, the latter being optionally combined with microparticles of a non-surfactant. As explained in this latter document, the formation of air bubbles in the solution results from the release of the air adsorbed on the surface of the particles, or trapped within the particle lattice, or caught between individual particles, this being so when the particles are agitated with the liquid carrier.

EP-A-013 1540 (Schering) also discloses the preparation of microbubbles suspensions in which a stabilized injectable carrier liquid, e.g., a physiological aqueous solution of salt, or a solution of a sugar like maltose, dextrose, lactose or galactose, without viscosity enhancer, is mixed with microparticles (in the 0.1 to 1 μm range) of the same sugars containing entrapped air. In order that the suspension of bubbles can develop within the liquid carrier, the foregoing documents recommend that both liquid and solid components be violently agitated together under sterile conditions; the agitation of both components together is performed for a few seconds and, once made, the suspension must then be used immediately, i.e., it should be injected within 5-10 minutes for echographic measurements; this indicates that the bubbles in the suspensions are not longlived and one practical problem with the use of microbubbles suspensions for injection is lack of stability with time. The present invention fully remedies this drawback.

In an attempt to cure the evanescence problem, microballoons, i.e., microvesicles with a material wall, have been developed. As said before, while the microbubbles only have an immaterial or evanescent envelope, i.e., they are only surrounded by a wall of liquid whose surface tension is being modified by the presence of a surfactant, the microballoons or microcapsules have a tangible envelope made of substantive material, e.g., a polymeric membrane with definite mechanical strength. In other terms, they are microvesicles of material in which the air or gas is more or less tightly encapsulated.

In U.S. Pat. No. 4,466,442 (Schering), there is disclosed a series of different techniques for producing suspensions of gas microbubbles in a liquid carrier liquid carrier using (a) a solution of a tenside (surfactant) in a carrier liquid (aqueous) and (b) a solution of a viscosity enhancer as stabilizer. For generating the bubbles, the techniques used there include forcing at high velocity a mixture of (a), (b) and air through a small aperture; or injecting (a) into (b) shortly before use together with a physiologically acceptable gas; or adding an acid to (a) and a carbonate to (b), both components being mixed together just before use and the acid reacting with the carbonate to generate CO₂ bubbles; or adding an over-pressurized gas to a mixture of (a) and (b) under storage, said gas being released into microbubbles at the time when the mixture is used for injection.

The tensides used in component (a) of U.S. Pat. No. 4,466,442 comprise lecithins; esters and ethers of fatty acids and fatty alcohols with polyoxyethylene and polyoxyethylated polyols like sorbitol, glycols and glycerol, cholesterol; and polyoxy-ethylene-polyoxypropylene polymers. The viscosity raising and stabilizing compounds include for instance mono- and polysaccharides (glucose, lactose, sucrose, dextran, sorbitol); polyols, e.g., glycerol, polyglycols; and polypeptides like proteins, gelatin, oxypolygelatin, plasma protein and the like.

In a typical preferred example of this latter document, equivalent volumes of (a) a 0.5% by weight aqueous solution of Pluronic® F-68 (a polyoxypropylene-polyoxyethylene polymer) and (b) a 10% lactose solution are vigorously shaken together under sterile conditions (closed vials) to provide a suspension of microbubbles ready for use as an ultrasonic contrast agent and lasting for at least 2 minutes. About 50% of the bubbles had a size below 50 μm.

Although the achievements of the prior art have merit, they suffer from several drawbacks which strongly limit their practical use by doctors and hospitals, namely their relatively short life-span (which makes test reproducibility difficult), relative low initial bubble concentration (the number of bubbles rarely exceeds 10⁴-10⁵ bubbles/ml and the count decreases rapidly with time) and poor reproducibility of the initial bubble count from test to test (which also makes comparisons difficult). Also it is admitted that for efficiently imaging certain organs, e.g., the left heart, bubbles smaller than 50 μm, preferably in the range of 0.5-10 μm, are required; with larger bubbles, there are risks of clots and consecutive emboly. Furthermore, the compulsory presence of solid microparticles or high concentrations of electrolytes and other relatively inert solutes in the carrier liquid may be undesirable physiologically in some cases.

The present invention concerns media adapted for injection into living bodies, e.g., for the purpose of ultrasonic echography and, more particularly, injectable liquid compositions comprising microbubbles or microballoons of air or physiologically acceptable gases as stable dispersions or suspensions in an aqueous liquid carrier. These compositions are mostly usable as contrast agents in ultrasonic echography to image the inside of blood-stream vessels and other cavities of living beings, e.g., human patients and animals. Other uses however are also contemplated as disclosed hereafter.

The invention also comprises dry compositions which, upon admixing with an aqueous carrier liquid, will generate the foregoing sterile suspension of microbubbles or microballoons thereafter usable as contrast agenta for ultrasonic echography and other purposes. The present invention also concerns stable dispersions or compositions of gas filled microvesicles in aqueous carrier liquids. These dispersions are generally usable for most kinds of applications requiring gases homogeneously dispersed in liquids. One notable application for such dispersions is to be injected into living beings, for instance for ultrasonic echography and other medical applications. The invention also concerns the methods for making the foregoing compositions including some materials involved in the preparations, for instance pressure-resistant gas-filled microbubbles, microcapsules and microballoons.

Stabilized Microbubble Compositions of the Invention

The term “lamellar form” defining the condition of at least a portion of the surfactant or surfactants of the present composition indicates that the surfactants, in strong contrast with the microparticles of the prior art (for instance EP-A-0123235), are in the form of thin films involving one or more molecular layers (in laminate form). Converting film forming surfactants into lamellar form can easily be done for instance by high pressure homogenization or by sonication under acoustical or ultrasonic frequencies. In this connection, it should be pointed out that the existence of liposomes is a well known and useful illustration of cases in which surfactants, more particularly lipids, are in lamellar form.

Liposome solutions are aqueous suspensions of microscopic vesicles, generally spherically shaped, which hold substances encapsulated therein. These vesicles are usually formed of one or more concentrically arranged layers (lamellae) of amphipathic compounds, i.e., compounds having a lipophobic hydrophilic moiety and a lipophilic hydrophobic moiety. See for instance “Liposome Methodology”, Ed. L. D. Leserman et al, Inserm 136, 2-8 (May 1982). Many surfactants or tensides, including lipids, particularly phospholipids, can be laminarized to correspond to this kind of structure. In this invention, one preferably uses the lipids commonly used for making liposomes, for instance the lecithins and other tensides disclosed in more detail hereafter, but this does in no way preclude the use of other surfactants provided they can be formed into layers or films.

It is important to note that no confusion should be made between the microbubbles of this invention and the disclosure of Ryan (U.S. Pat. No. 4,900,540) reporting the use of air or gas filled liposomes for echography. In this method Ryan encapsulates air or a gas within liposomic vesicles; in embodiments of the present invention microbubbles of air or a gas are formed in a suspension of liposomes (i.e., liquid filled liposomes) and the liposomes apparently stabilize the microbubbles. In Ryan, the air is inside the liposomes, which means that within the bounds of the presently used terminology, the air filled liposomes of Ryan belong to the class of microballoons and not to that of the microbubbles.

Practically, to achieve the suspensions of microbubbles according to the invention, one may start with liposomes suspensions or solutions prepared by any technique reported in the prior art, with the obvious difference that in the present case the liposomic vesicles are preferably “unloaded”, i.e., they do not need to keep encapsulated therein any foreign material other than the liquid of suspension as is normally the object of classic liposomes. Hence, preferably, the liposomes of the present invention will contain an aqueous phase identical or similar to the aqueous phase of the solution itself. Then air or a gas is introduced into the liposome solution so that a suspension of microbubbles will form, said suspension being stabilized by the presence of the surfactants in lamellar form. Notwithstanding, the material making the liposome walls can be modified within the scope of the present invention, for instance by covalently grafting thereon foreign molecules designed for specific purposes as will be explained later.

The preparation of liposome solutions has been abundantly discussed in many publications, e.g., U.S. Pat. No. 4,224,179 and WO-A-88/09165 and all citations mentioned therein. This prior art is used here as reference for exemplifying the various methods suitable for converting film forming tensides into lamellar form. Another basic reference by M. C. Woodle and D. Papahadjopoulos is found in “Methods in Enzymology” 171 (1989), 193.

For instance, in a method disclosed in D. A. Tyrrell et al, Biochimica & Biophysica Acta 457 (1976), 259-302, a mixture of a lipid and an aqueous liquid carrier is subjected to violent agitation and thereafter sonicated at acoustic or ultrasonic frequencies at room or elevated temperature. In the present invention, it has been found that sonication without agitation is convenient. Also, an apparatus for making liposomes, a high pressure homogenizer such as the Microfluidizer®, which can be purchased from Microfluidics Corp., Newton, Mass. 02164 USA, can be used advantageously. Large volumes of liposome solutions can be prepared with this apparatus under pressures which can reach 600-1200 bar.

In another method, according to the teaching of GB-A-2,134,869 (Squibb), microparticles (10 μm or less) of a hydrosoluble carrier solid (NaCl, sucrose, lactose and other carbohydrates) are coated with an amphipathic agent; the dissolution of the coated carrier in an aqueous phase will yield liposomic vesicles. In GB-A-2,135,647 insoluble particles, e.g., glass or resin microbeads are coated by moistening in a solution of a lipid in an organic solvent followed by removal of the solvent by evaporation. The lipid coated microbeads are thereafter contacted with an aqueous carrier phase, whereby liposomic vesicles will form in that carrier phase.

The introduction of air or gas into a liposome solution in order to form therein a suspension of microbubbles can be effected by usual means, inter alia by injection, that is, forcing said air or gas through tiny orifices into the liposome solution, or simply dissolving the gas in the solution by applying pressure and thereafter suddenly releasing the pressure. Another way is to agitate or sonicate the liposome solution in the presence of air or an entrappable gas. Also one can generate the formation of a gas within the solution of liposomes itself, for instance by a gas releasing chemical reaction, e.g., decomposing a dissolved carbonate or bicarbonate by acid. The same effect can be obtained by dissolving under pressure a low boiling liquid, for instance butane, in the aqueous phase and thereafter allowing said liquid to boil by suddenly releasing the pressure.

Notwithstanding, an advantageous method is to contact the dry surfactant in lamellar or thin film form with air or an adsorbable or entrappable gas before introducing said surfactant into the liquid carrier phase. In this regard, the method can be derived from the technique disclosed in GB-A-2,135,647, i.e., solid microparticles or beads are dipped in a solution of a film forming surfactant (or mixture of surfactants) in a volatile solvent, after which the solvent is evaporated and the beads are left in contact with air (or an adsorbable gas) for a time sufficient for that air to become superficially bound to the surfactant layer. Thereafter, the beads coated with air filled surfactant are put into a carrier liquid, usually water with or without additives, whereby air bubbles will develop within the liquid by gentle mixing, violent agitation being entirely unnecessary. Then the solid beads can be separated, for instance by filtration, from the microbubble suspension which is remarkably stable with time.

Needless to say that, instead of insoluble beads or spheres, one may use as supporting particles water soluble materials like that disclosed in GB-A-2,134,869 (carbohydrates or hydrophilic polymers), whereby said supporting particles will eventually dissolve and final separation of a solid becomes unnecessary. Furthermore in this case, the material of the particles can be selected to eventually act as stabilizer or viscosity enhancer wherever desired.

In a variant of the method, one may also start with dehydrated liposomes, i.e., liposomes which have been prepared normally by means of conventional techniques in the form of aqueous solutions and thereafter dehydrated by usual means, e.g., such as disclosed in U.S. Pat. No. 4,229,360 also incorporated herein by reference. One of the methods for dehydrating liposomes recommended in this reference is freeze-drying (lyophilization), i.e., the liposome solution is frozen and dried by evaporation (sublimation) under reduced pressure. Prior to effecting freeze-drying, a hydrophilic stabilizer compound is dissolved in the solution, for instance a carbohydrate like lactose or sucrose or a hydrophilic polymer like dextran, starch, PVP, PVA and the like. This is useful in the present invention since such hydrophilic compounds also aid in homogenizing the microbubbles size distribution and enhance stability under storage. Actually making very dilute aqueous solutions (0. 1 -10% by weight) of freeze-dried liposomes stabilized with, for instance, a 5:1 to 10:1 weight ratio of lactose to lipid enables to produce aqueous microbubbles suspensions counting 10⁸-10⁹ microbubbles/ml (size distribution mainly 0.5-10 μm) which are stable for at least a month (and probably much longer) without significant observable change. And this is obtained by simple dissolution of the air-stored dried liposomes without shaking or any violent agitation. Furthermore, the freeze-drying technique under reduced pressure is very useful because it permits, after drying, to restore the pressure above the dried liposomes with any entrappable gas, i.e., nitrogen, CO₂, argon, methane, freon, etc., whereby after dissolution of the liposomes processed under such conditions suspensions of microbubbles containing the above gases are obtained.

Microbubbles suspensions formed by applying gas pressure on a dilute solution of laminated lipids in water (0.1-10% by weight) and thereafter suddenly releasing the pressure have an even higher bubble concentration, e.g., in the order of 10¹⁰-10¹¹ bubbles/ml. However, the average bubble size is somewhat above 10 μm, e.g., in the 10-50 μm range. In this case, bubble size distribution can be narrowed by centrifugation and layer decantation.

The tensides or surfactants which are convenient in this invention can be selected from all amphipathic compounds capable of forming stable films in the presence of water and gases. The preferred surfactants which can be laminarized include the lecithins (phosphatidyl-choline) and other phospholipids, inter alia phosphatidic acid (PA), phosphatidylinositol, phosphatidylethanolamine (PE), phosphatidylserine (PS), phosphatidylglycerol (PG), cardiolipin (CL), sphingomyelins, the plasmogens, the cerebrosides, etc. Examples of suitable lipids are the phospholipids in general, for example, natural lecithins, such as egg lecithin or soya bean lecithin, or synthetic lecithins such as saturated synthetic lecithins, for example, dimyristoylphosphatidylcholine, dipalmitoylphosphatidylcholine or distearoylphosphatidylcholine or unsaturated synthetic lecithins, such as dioleylphosphatidylcholine or dilinoleylphosphatidylcholine, with egg lecithin or soya bean lecithin being preferred. Additives like cholesterol and other substances (see below) can be added to one or more of the foregoing lipids in proportions ranging from zero to 50% by weight.

Such additives may include other surfactants that can be used in admixture with the film forming surfactants and most of which are recited in the prior art discussed in the introduction of this specification. For instance, one may cite free fatty acids, esters of fatty acids with polyoxyalkylene compounds like polyoxypropylene glycol and polyoxyalkylene glycol; ethers of fatty alcohols with polyoxyalkylene glycols; esters of fatty acids with polyoxyalklated sorbitan; soaps; glycerol-polyalkylene stearate; glycerol-polyoxyethylene ricinoleate; homo- and copolymers of polyalkylene glycols; polyethoxylated soya-oil and castor oil as well as hydrogenated derivatives; ethers and esters of sucrose or other carbohydrates with fatty acids, fatty alcohols, these being optionally polyoxyalkylated; mono- di and triglycerides of saturated or unsaturated fatty acids; glycerides of soya-oil and sucrose. The amount of the non-film forming tensides or surfactants can be up to 50% by weight of the total amount of surfactants in the composition but is preferably between zero and 30%.

The total amount of surfactants relative to the aqueous carrier liquid is best in the range of 0.01 to 25% by weight but quantities in the range 0.5-5% are advantageous because one always tries to keep the amount of active substances in an injectable solution as low as possible, this being to minimize the introduction of foreign materials into living beings even when they are harmless and physiologically compatible.

Further optional additives to the surfactants include:

a) substances which are known to provide a negative charge on liposomes, for example, phosphatidic acid, phosphatidyl-glycerol or dicetyl phosphate;

b) substances known to provide a positive charge, for example, stearyl amine, or stearyl amine acetate;

c) substances known to affect the physical properties of the lipid films in a more desirable way; for example, capro-lactam and/or sterols such as cholesterol, ergosterol, phytosterol, sitosterol, sitosterol pyroglutamate, 7-dehydro-cholesterol or lanosterol, may affect lipid films rigidity;

d) substances known to have antioxidant properties to improve the chemical stability of the components in the suspensions, such as tocopherol, propyl gallate, ascorbyl palmitate, or butylated hydroxy toluene.

The aqueous carrier in this invention is mostly water with possibly small quantities of physiologically compatible liquids such as isopropanol, glycerol, hexanol and the like (see for instance EP-A-052575). In general the amount of the organic hydrosoluble liquids will not exceed 5-10% by weight.

The present composition may also contain dissolved or suspended therein hydrophilic compounds and polymers defined generally under the name of viscosity enhancers or stabilizers. Although the presence of such compounds is not compulsory for ensuring stability to the air or gas bubbles with time in the present dispersions, they are advantageous to give some kind of “body” to the solutions. When desired, the upper concentrations of such additives when totally innocuous can be very high, for instance up to 80-90% by weight of solution with Iopamidol and other iodinated X-ray contrast agents. However, with the viscosity enhancers like for instance sugars, e.g., lactose, sucrose, maltose, galactose, glucose, etc. or hydrophilic polymers like starch, dextran, polyvinyl alcohol, polyvinyl-pyrrolidone, dextrin, xanthan or partly hydrolyzed cellulose oligomers, as well as proteins and polypeptides, the concentrations are best between about 1 and 40% by weight, a range of about 5-20% being preferred.

Like in the prior art, the injectable compositions of this invention can also contain physiologically acceptable electrolytes; an example is an isotonic solution of salt.

The present invention naturally also includes dry storable pulverulent blends which can generate the present microbubble containing dispersions upon simple admixing with water or an aqueous carrier phase. Preferably such dry blends or formulations will contain all solid ingredients necessary to provide the desired microbubbles suspensions upon the simple addition of water, i.e., principally the surfactants in lamellar form containing trapped or adsorbed therein the air or gas required for microbubble formation, and accessorily the other non-film forming surfactants, the viscosity enhancers and stabilizers and possibly other optional additives. As said before, the air or gas entrapment by the laminated surfactants occurs by simply exposing said surfactants to the air (or gas) at room or superatmospheric pressure for a time sufficient to cause said air or gas to become entrapped within the surfactant. This period of time can be very short, e.g., in the order of a few seconds to a few minutes although over-exposure, i.e., storage under air or under a gaseous atmosphere is in no way harmful. What is important is that air can well contact as much as possible of the available surface of the laminated surfactant, i.e., the dry material should preferably be in a “fluffy” light flowing condition. This is precisely this condition which results from the freeze-drying of an aqueous solution of liposomes and hydrophilic agent as disclosed in U.S. Pat. No. 4,229,360.

In general, the weight ratio of surfactants to hydrophilic viscosity enhancer in the dry formulations will be in the order of 0.1:10 to 10: 1, the further optional ingredients, if any, being present in a ratio not exceeding 50% relative to the total of surfactants plus viscosity enhancers.

The dry blend formulations of this invention can be prepared by very simple methods. As seen before, one preferred method is to first prepare an aqueous solution in which the film forming lipids are laminarized, for instance by sonication, or using any conventional technique commonly used in the liposome field, this solution also containing the other desired additives, i.e., viscosity enhancers, non-film forming surfactants, electrolyte, etc., and thereafter freeze drying to a free flowable powder which is then stored in the presence of air or an entrappable gas.

The dry blend can be kept for any period of time in the dry state and sold as such. For putting it into use, i.e., for preparing a gas or air microbubble suspension for ultrasonic imaging, one simply dissolves a known weight of the dry pulverulent formulation in a sterile aqueous phase, e.g., water or a physiologically acceptable medium. The amount of powder will depend on the desired concentration of bubbles in the injectable product, a count of about 10⁸-10⁹ bubbles/ml being generally that from making a 5-20% by weight solution of the powder in water. But naturally this figure is only indicative, the amount of bubbles being essentially dependent on the amount of air or gas trapped during manufacture of the dry powder. The manufacturing steps being under control, the dissolution of the dry formulations will provide microbubble suspensions with well reproducible counts.

The resulting microbubble suspensions (bubble in the 0.5-10 μm range) are extraordinarily stable with time, the count originally measured at start staying unchanged or only little changed for weeks and even months; the only observable change is a kind of segregation, the larger bubbles (around 10 μm) tending to rise faster than the small ones.

It has also been found that the microbubbles suspensions of this invention can be diluted with very little loss in the number of microbubbles to be expected from dilution, i.e., even in the case of high dilution ratios, e.g., 1/10² to 1/10⁴, the microbubble count reduction accurately matches with the dilution ratio. This indicates that the stability of the bubbles depends on the surfactant in lamellar form rather than on the presence of stabilizers or viscosity enhancers like in the prior art. This property is advantageous in regard to imaging test reproducibility as the bubbles are not affected by dilution with blood upon injection into a patient.

Another advantage of the bubbles of this invention versus the microbubbles of the prior art surrounded by a rigid but breakable membrane which may irreversibly fracture under stress is that when the present suspensions are subject to sudden pressure changes, the present bubbles will momentarily contract elastically and then resume their original shape when the pressure is released. This is important in clinical practice when the microbubbles are pumped through the heart and therefore are exposed to alternating pressure pulses.

The reasons why the microbubbles in this invention are so stable are not clearly understood. Since to prevent bubble escape the buoyancy forces should equilibrate with the retaining forces due to friction, i.e., to viscosity, it is theorized that the bubbles are probably surrounded by the laminated surfactant. Whether this laminar surfactant is in the form of a continuous or discontinuous membrane, or even as closed spheres attached to the microbubbles, is for the moment unknown but under investigation. However the lack of a detailed knowledge of the phenomena presently involved does not prelude full industrial operability of the present invention.

The bubble suspensions of the present invention are also useful in other medical/diagnostic applications where it is desirable to target the stabilized microbubbles to specific sites in the body following their injection, for instance to thrombi present in blood vessels, to atherosclerotic lesions (plaques) in arteries, to tumor cells, as well as for the diagnosis of altered surfaces of body cavities, e.g., ulceration sites in the stomach or tumors of the bladder. For this, one can bind monoclonal antibodies tailored by genetic engineering, antibody fragments or polypeptides designed to mimic antibodies, bioadhesive polymers, lectins and other site-recognizing molecules to the surfactant layer stabilizing the microbubbles. Thus monoclonal antibodies can be bound to phospholipid bilayers by the method described by L. D. Leserman, P. Machy and J. Barbet (“Liposome Technology vol. III” p. 29 ed. by G. Gregoriadis, CRC Press 1984). In another approach a palmitoyl antibody is first synthesized and then incorporated in phospholipid bilayers following L. Huang, A. Huang and S. J. Kennel (“Liposome Technology vol. III” p. 51 ed. by G. Gregoriadis, CRC Press 1984). Alternatively, some of the phospholipids used in the present invention can be carefully selected in order to obtain preferential uptake in organs or tissues or increased half-life in blood. Thus GM1 gangliosides- or phosphatidylinositol-containing liposomes, preferably in addition to cholesterol, will lead to increased, half-lifes in blood after intravenous administration in analogy with A. Gabizon, D. Papahadjopoulos, Proc. Natl Acad. Sci USA 85 (1988) 6949.

The gases in the microbubbles of the present invention can include, in addition to current innocuous physiologically acceptable gases like CO₂, nitrogen, N₂O, methane, butane, freon and mixtures thereof, radioactive gases such as ¹³³Xe or ⁸¹Kr are of particular interest in nuclear medicine for blood circulation measurements, for lung scintigraphy etc.

The invention described up until this point can be further elucidated by the description of the following representative (but not limiting) embodiments, numbered 1-27:

1. A composition adapted for injection into the bloodstream and body cavities of living beings, e.g., for the purpose of ultrasonic echography consisting of a suspension of air or gas microbubbles in a physiologically acceptable aqueous carrier phase comprising from about 0.01 to about 20% by weight of one or more dissolved or dispersed surfactants, characterized in that at least one of the surfactants is a film forming surfactant present in the composition at least partially in lamellar or laminar form.

2. The composition of embodiment 1, characterized in that the lamellar surfactant is in the form of mono- or pluri-molecular membrane layers.

3. The composition of embodiment 1, characterized in that the lamellar surfactant is in the form of liposome vesicles.

4. The composition of embodiment 1, characterized in that it essentially consists of a liposome solution containing air or gas microbubbles developed therein.

5. The composition of embodiment 4, characterized in that the size of most of both liposomes and microbubbles is below 50 μm, preferably below 10 μm.

6. The composition of embodiment 1, containing about 10⁸-10⁹ bubbles of 0.5-10 μm size/ml, said concentration showing little or substantially no variability under storage for at least a month.

7. The composition of embodiment 1, characterized in that the surfactants are selected from phospholipids including the lecithins such as phosphatidic acid, phosphatidylcholine, phosphatidylethanolamine, phosphatidylserine, phosphatidylglycerol, phosphatidylinositol, cardiolipin and sphyngomyelin.

8. The composition of embodiment 7, characterized in further containing substances affecting the properties of liposomes selected form phosphatidylglycerol, dicetylphosphate, cholesterol, ergosterol, phytosterol, sitosterol, lanosterol, tocopterol, propyl gallate, ascorbyl palmitate and butylated hydroxytoluene.

9. The composition of embodiment 1, further containing dissolved viscosity enhancers or stabilizers selected from linear and cross-linked poly- and oligo-saccharides, sugars, hydrophilic polymers and iodinated compounds such as Iopamidol in a weight ratio to the surfactants comprised between about 1:5 to 100:1.

10. The composition of embodiment 1, in which the surfactants comprise up to 50% by weight of non-laminar surfactants selected from fatty acids, esters, and ethers of fatty acids and alcohols with polyols such as polyalkylene glycols, polyalkylenated sugars and other carbohydrates, and polyalkylenated glycerol.

11. A method for the preparation of the suspensions of embodiment 1, characterized by the following steps:

(a) selecting at least one film forming surfactant and converting it into lamellar form;

(b) contacting the surfactant in lamellar form with air or an adsorbable or entrappable gas for a time sufficient for that air or gas to become bound by said surfactant; and

(c) admixing the surfactant in lamellar form with an aqueous liquid carrier, whereby a stable dispersion of air or gas microbubbles in said liquid carrier will result.

12. The method of embodiment 11, in which step (c) is brought about before step (b), the latter being effected by introducing pressurized air or gas into the liquid carrier and thereafter releasing the pressure.

13. The method of embodiment 11, in which step (c) is brought about by gentle mixing of the components, no shaking being necessary, whereby the air or gas bound to the lamellar surfactant in step (b) will develop into a suspension of stable microbubbles.

14. The method of embodiments 11 or 12, in which the liquid carrier contains dissolved therein stabilizer compounds selected from hydrosoluble proteins, polypeptides, sugars, poly- and oligo-saccharides and hydrophilic polymers.

15. The method of embodiment 11, in which the conversion of step (a) is effected by coating the surfactant onto particles of soluble or insoluble materials; step (b) is effected by letting the coated particles stand for a while under air or a gas; and step (c) is effected by admixing the coated particles with an aqueous liquid carrier.

16. The method of embodiment 11, in which the conversion of step (a) is effected by sonicating or homogenizing under high pressure an aqueous solution of film forming lipids, this operation leading, at least partly, to the formation of liposomes.

17. The method of embodiment 16, in which step (b) is effected by freeze-drying the liposome containing solution, the latter optionally containing hydrophilic stabilizers and contacting the resulting freeze-dried product with air or gas for a period of time.

18. The method of embodiments 16 and 17, in which the water solution of film forming lipids also contains viscosity enhancers or stabilizers selected from hydrophilic polymers and carbohydrates in weight ratio relative to the lipids comprised between 1:5 and 100:1.

19. A dry pulverulent formulation which, upon dissolution in water, will form an aqueous suspension of microbubbles for ultrasonic echography, characterized in containing one or more film forming surfactants in laminar form and hydrosoluble stabilizers.

20. The dry formulation of embodiment 19, in which the surfactants in laminar form are in the form of fine layers deposited on the surface of soluble or insoluble solid particulate material.

21. The dry formulation of embodiment 20, in which the insoluble solid particles are glass or polymer beads.

22. The dry formulation of embodiment 20, in which the soluble particles are made of hydrosoluble carbohydrates, polysaccharides, synthetic polymers, albumin, gelatin or Iopamidol.

23. The dry formulation of embodiment 19, which comprises freeze-dried liposomes.

24. The use of the injectable composition of embodiment 1 for ultrasonic echography.

25. The use of the injectable composition of embodiments 1-10 for transporting in the blood-stream or body cavities bubbles of foreign gases active therapeutically or diagnostically.

26. The composition of embodiment 4, in which the surfactant comprises, bound thereto, bioactive species designed for specific targeting purposes, e.g., for immobilizing the bubbles in specifically defined sites in the circulatory system, or in organs, or in tissues.

27. The composition of embodiment 4, in which the surfactant comprises, bound thereto, bioactive species selected from monoclonal antibodies, antibody fragments or polypeptides designed to mimic antibodies, bioadhesive polymers, lectins and other receptor recognizing molecules.

The following Examples further illustrate the invention from a practical standpoint.

Echogenic Measurements

Echogenicity measurements were performed in a pulse-echo system made of a plexiglas specimen holder (diameter 30 mm) and a transducer holder immersed in a constant temperature water bath, a pulser-receiver (Accutron M3010S) with for the receiving part an external pre-amplifier with a fixed gain of 40 dB and an internal amplifier with adjustable gain from −40 to +40 dB. A 10 MHz low-pass filter was inserted in the receiving part to improve the signal to noise ratio. The A/D board in the IBM PC was a Sonotek STR 832. Measurements were carried out at 2.25, 3.5, 5 and 7.5 MHz.

### EXAMPLE 1

A liposome solution (50 mg lipids per ml) was prepared in distilled water by the REV method (see F. Szoka Jr. and D. Papahadjopoulos, Proc. Natl. Acad. Sci. USA 75 (1978) 4194) using hydrogenated soya lecithin (NC 95 H, Nattermann Chemie, Koln, W. Germany) and dicetylphosphate in a molar ratio 9/1. This liposome preparation was extruded at 65° C. (to calibrate the vesicle size) through a 1 μm polycarbonate filter (Nucleopore). Two ml of this solution were admixed with 5 ml of a 75% iopamidol solution in water and 0.4 ml of air and the mixture was forced back and forth through a two syringe system as disclosed in DE-A-3529195, while maintaining continuously a slight over-pressure. This resulted in the formation of a suspension of microbubbles of air in the liquid (10⁵-10⁶ bubbles per ml, bubble size 1-20 μm as estimated by light microscopy) which was stable for several hours at room temperature. This suspension gave a strong echo signal when tested by ultrasonic echography at 7.5, 5, 3.5 and 2.25 MHz.

### EXAMPLE 2

A distilled water solution (100 ml) containing by weight 2% of hydrogenated soya lecithin and dicetylphosphate in a 9/1 molar ratio was sonicated for 15 min at 60°-65° C. with a Branson probe sonifier (Type 250). After cooling, the solution was centrifuged for 15 min at 10,000 g and the supernatant was recovered and lactose added to make a 7.5% b.w. solution. The solution was placed in a tight container in which a pressure of 4 bar of nitrogen was established for a few minutes while shaking the container. Afterwards, the pressure was released suddenly whereby a highly concentrated bubble suspension was obtained (10¹⁰-10¹¹ bubbles/ml). The size distribution of the bubbles was however wider than in Example 1, i.e., from about 1 to 50 μm. The suspension was very stable but after a few days a segregation occurred in the standing phase, the larger bubbles tending to concentrate in the upper layers of the suspension.

### EXAMPLE 3

Twenty g of glass beads (diameter about 1 mm) were immersed into a solution of 100 mg of dipalmitoylphosphatidylcholine (Fluka A. G. Buchs) in 10 ml of chloroform. The beads were rotated under reduced pressure in a rotating evaporator until all CHCl³ had escaped. Then the beads were further rotated under atmospheric pressure for a few minutes and 10 ml of distilled water were added. The beads were removed and a suspension of air microbubbles was obtained which was shown to contain about 10⁶ bubbles/ml after examination under the microscope. The average size of the bubbles was about 3-5 μm. The suspension was stable for several days at least.

### EXAMPLE 4

A hydrogenated soya lecithin/dicetylphosphate suspension in water was laminarized using the REV technique as described in Example 1. Two ml of the liposome preparation were added to 8 ml of 15% maltose solution in distilled water. The resulting solution was frozen at −30° C., then lyophilized under 0.1 Torr. Complete sublimation of the ice was obtained in a few hours. Thereafter, air pressure was restored in the evacuated container so that the lyophilized powder became saturated with air in a few minutes.

The dry powder was then dissolved in 10 ml of sterile water under gentle mixing, whereby a microbubble suspension (10⁸-10⁹ microbubbles per ml, dynamic viscosity <20 mPa.s) was obtained. This suspension containing mostly bubbles in the 1-5 μm range was stable for a very long period, as numerous bubbles could still be detected after 2 months standing. This microbubble suspension gave a strong response in ultrasonic echography. If in this example the solution is frozen by spraying in air at −30° to −70° C. to obtain a frozen snow instead of a monolithic block and the snow is then evaporated under vacuum, excellent results are obtained.

### EXAMPLE 5

Two ml samples of the liposome solution obtained as described in Example 4 were mixed with 10 ml of an 5% aqueous solution of gelatin (sample 5A), human albumin (sample 5B), dextran (sample 5C) and iopamidol (sample 5D). All samples were lyophilized. After lyophilization and introduction of air, the various samples were gently mixed with 20 ml of sterile water. In all cases, the bubble concentration was above 10⁸ bubbles per ml and almost all bubbles were below 10 μm. The procedure of the foregoing Example was repeated with 9 ml of the liposome preparation (450 mg of lipids) and only one ml of a 5% human albumin solution. After lyophilization, exposure to air and addition of sterile water (20 ml), the resulting solution contained 2×10⁸ bubbles per ml, most of the them below 10 μm.

### EXAMPLE 6

Lactose (500 mg), finely milled to a particle size of 1-3 μm, was moistened with a chloroform (5 ml) solution of 100 mg of dimyristoylphosphatidylcholine/cholesterol/dipalmitoylphosphatidic acid (from Fluka) in a molar ratio of 4:1:1 and thereafter evaporated under vacuum in a rotating evaporator. The resulting free flowing white powder was rotated a few minutes under nitrogen at normal pressure and thereafter dissolved in 20 ml of sterile water. A microbubble suspension was obtained with about 10⁵-10⁶ microbubbles per ml in the 1-10 μm size range as ascertained by observation under the microscope. In this Example, the weight ratio of coated surfactant to water-soluble carrier was 1:5. Excellent results ( 10⁷-10⁸ microbubbles/ml) are also obtained when reducing this ratio to lower values, i.e., down to 1:20, which will actually increases the surfactant efficiency for the intake of air, that is, this will decrease the weight of surfactant necessary for producing the same bubble count.

### EXAMPLE 7

An aqueous solution containing 2% of hydrogenated soya lecithin and 0.4% of Pluronic® F68 (a non ionic polyoxyethylenepolyoxypropylene copolymer surfactant) was sonicated as described in Example 2. After cooling and centrifugation, 5 ml of this solution were added to 5 ml of a 15% maltose solution in water. The resulting solution was frozen at −30° C. and evaporated under 0.1 Torr. Then air pressure was restored in the vessel containing the dry powder. This was left to stand in air for a few seconds, after which it was used to make a 10% by weight aqueous solution which showed under the microscope to be a suspension of very tiny bubbles (below 10 μm); the bubble concentration was in the range of 10⁷ bubbles per ml. This preparation gave a very strong response in ultrasonic echography at 2.25, 3.5, 5 and 7.5 MHz.

### EXAMPLE 8

Two-dimensional echocardiography was performed in an experimental dog following peripheral vein injection of 0.1-2 ml of the preparation obtained in Example 4. Opacification of the left heart with clear outlining of the endocardium was observed, thereby confirming that the microbubbles (or at least a significant part of them) were able to cross the pulmonary capillary circulation.

### EXAMPLE 9

A phospholipid/maltose lyophilized powder was prepared as described in Example 4. However, at the end of the lyophilization step, a ¹³³Xe containing gas mixture was introduced in the evacuated container instead of air. A few minutes later, sterile water was introduced and after gentle mixing a microbubble suspension containing ¹³³Xe in the gas phase was produced. This microbubble suspension was injected into living bodies to undertake investigations requiring use of ¹³³Xe as tracer. Excellent results were obtained.

### EXAMPLE 10 (COMPARATIVE)

In U.S. Pat. No. 4,900,540, Ryan et al disclose gas filled liposomes for ultrasonic investigations. According to the citation, liposomes are formed by conventional means but with the addition of a gas or gas precursor in the aqueous composition forming the liposome core (col. 2, lines 15-27).

Using a gas precursor (bicarbonate) is detailed in Examples 1 and 2 of the reference. Using an aqueous carrier with an added gas for encapsulating the gas in the liposomes (not exemplified by Ryan et al) will require that the gas be in the form of very small bubbles, i.e., of size similar or smaller than the size of the liposome vesicles.

Aqueous media in which air can be entrapped in the form of very small bubbles (2.5-5 μm) are disclosed in M. W. Keller et al, J. Ultrasound Med. 5 (1986), 413-498.

A quantity of 126 mg of egg lecithin and 27 mg of cholesterol were dissolved in 9 ml of chloroform in a 200 ml round bottom flask. The solution of lipids was evaporated to dryness on a Rotavapor whereby a film of the lipids was formed on the walls of the flask. A 10 ml of a 50% by weight aqueous dextrose solution was sonicated for 5 min according to M. W. Keller et al (ibid) to generate air microbubbles therein and the sonicated solution was added to the flask containing the film of lipid, whereby hand agitation of the vessel resulted into hydration of the phospholipids and formation of multilamellar liposomes within the bubbles containing carrier liquid.

After standing for a while, the resulting liposome suspension was subjected to centrifugation under 5000 g for 15 min to remove from the carrier the air not entrapped in the vesicles. It was also expected that during centrifugation, the air filled liposomes would segregate to the surface by buoyancy.

After centrifugation the tubes were examined and showed a bottom residue consisting of agglomerated dextrose filled liposomes and a clear supernatant liquid with substantially no bubbles left. The quantity of air filled liposomes having risen by buoyancy was negligibly small and could not be ascertained.

### EXAMPLE 11 (COMPARATIVE)

An injectable contrast composition was prepared according to Ryan (U.S. Pat. No. 4,900,540, col. 3, Example 1). Egg lecithin (126 mg) and cholesterol (27 mg) were dissolved in 9 ml of diethylether. To the solution were added 3 ml of 0.2 molar aqueous bicarbonate and the resulting two phase systems was sonicated until becoming homogeneous. The mixture was evaporated in a Rotavapor apparatus and 3 ml of 0.2 molar aqueous bicarbonate were added.

A 1 ml portion of the liposome suspension was injected into the jugular vein of an experimental rabbit, the animal being under condition for heart ultrasonic imaging using an Acuson 128-XP5 ultrasonic imager (7.5 transducer probe for imaging the heart). The probe provided a cross-sectional image of the right and left ventricles (mid-papillary muscle). After injection, a light and transient (a few seconds) increase in the outline of the right ventricle was observed. The effect was however much inferior to the effect observed using the preparation of Example 4. No improvement of the imaging of the left ventricle was noted which probably indicates that the CO₂ loaded liposomes did not pass the pulmonary capillaries barrier.

### Stable Microballoon Compositions of the Invention

Desirable features have also now been achieved with the microballoons of the present invention which are of micronic or submicronic size bounded by a polymer membrane filled with air or a gas suitable, when in the form of suspensions in a liquid carrier, to be administered to human or animal patients for therapeutic or diagnostic applications, e.g., for the purpose of ultrasonic echography imaging. The polymer of the membrane is a deformable and resilient interfacially deposited polymer. The invention also includes air or gas filled microballoons bounded by an elastic interfacial polymeric membrane adapted to form with suitable physiologically acceptable aqueous carrier liquids suspensions to be taken orally, rectally and urethrally, or injectable into living organisms for therapeutic or diagnostic purposes. These microballoons are characterized as being non-coalescent, dry and instantly dispersible by admixing with a liquid carrier. Moreover, although the present microspheres can generally be made relatively short-lived, i.e., susceptible to biodegradation to cope with the foregoing metabolization problems by using selected types of polymers, this feature (which is actually controlled by the fabrication parameters) is not a commercial drawback because either the microballoons can be stored and shipped dry, a condition in which they are stable indefinitely, or the membrane can be made substantially impervious to the carrier liquid, degradation starting to occur only after injection. In the first case, the microballoons supplied in dry powder form are simply admixed with a proportion of an aqueous phase carrier before use, this proportion being selected depending on the needs. Note that this is an additional advantage over the prior art products because the concentration can be chosen at will and initial values far exceeding the aforementioned 10⁸/ml, i.e., in the range 10⁵ to 10¹⁰, are readily accessible. It should be noted that the method of the invention (to be disclosed hereafter) enables to control porosity to a wide extent; hence microballoons with a substantially impervious membrane can be made easily which are stable in the form of suspensions in aqueous liquids and which can be marketed as such also.

Microspheres with membranes of interfacially deposited polymers, although in the state where they are filled with liquid, are well known in the art. They may normally result from the emulsification into droplets (the size of which is controllable in function to the emulsification parameters) of a first aqueous phase in an organic solution of polymer followed by dispersion of this emulsion into a second water phase and subsequent evaporation of the organic solvent. During evaporation of the volatile solvent, the polymer deposits interfacially at the droplets boundary and forms a microporous membrane which efficiently bounds the encapsulated first aqueous phase from the surrounding second aqueous phase. This technique, although possible, is not preferred in the present invention.

Alternatively, one may emulsify with an emulsifier a hydrophobic phase in an aqueous phase (usually containing viscosity increasing agents as emulsion stabilizers) thus obtaining an oil-in-water type emulsion of droplets of the hydrophobic phase and thereafter adding thereto a membrane forming polymer dissolved in a volatile organic solvent not miscible with the aqueous phase.

If the polymer is insoluble in the hydrophobic phase, it will deposit interfacially at the boundary between the droplets and the aqueous phase. Otherwise, evaporation of the volatile solvent will lead to the formation of said interfacially deposited membrane around the droplets of the emulsified hydrophobic phase. Subsequent evaporation of the encapsulated volatile hydrophobic phase provides water filled microspheres surrounded by interfacially deposited polymer membranes. This technique which is advantageously used in the present invention is disclosed by K. Uno et al. in J. Microencapsulation 1 (1984), 3-8 and K. Makino et al., Chem. Pharm. Bull. 33 (1984), 1195-1201. As said before, the size of the droplets can be controlled by changing the emulsification parameters, i.e., nature of emulsifier (more effective the surfactant, i.e., the larger the hydrophilic to lipophilic balance, the smaller the droplets) and the stirring conditions (faster and more energetic the agitation, the smaller the droplets).

In another variant, the interfacial wall forming polymer is dissolved in the starting hydrophobic phase itself, the latter is emulsified into droplets in the aqueous phase and the membrane around the droplets will form upon subsequent evaporation of this encapsulated hydrophobic phase. An example of this is reported by J. R. Farnand et el., Powder Technology 22 (1978), 11-16 who emulsify a solution of polymer (e.d., polyethylene) in naphthalene in boiling water, then after cooling they recover the naphthalene in the form of a suspension of polymer bounded microbeads in cold water and, finally, they remove the naphthalene by subjecting the microbeads to sublimation, whereby 25 micron microballoons are produced. Other examples exist, in which a polymer is dissolved in a mixed hydrophobic phase comprising a volatile hydrophobic organic solvent and a water-soluble organic solvent, then this polymer solution is emulsified in a water phase containing an emulsifier, whereby the water-soluble solvent disperses into the water phase, thus aiding in the formation of the emulsion of microdroplets of the hydrophobic phase and causing the polymer to precipitate at the interface; this is disclosed in EP-A-274,961 (H. Fessi).

The aforementioned techniques can be adapted to the preparation of air or gas filled microballoons suited for ultrasonic imaging provided that appropriate conditions are found to control sphere size in the desired ranges, cell-wall permeability or imperviousness and replacement of the encapsulated liquid phase by air or a selected gas. Control of overall sphere size is obviously important to adapt the microballoons to use purposes, i.e., injection or oral intake. The size conditions for injection (about 0.5-10 μm average size) have been discussed previously. For oral application, the range can be much wider, being considered that echogenicity increases with size; hence microballoons in several size ranges between say 1 and 1000 microns can be used depending on the needs and provided the membrane is elastic enough not to break during transit in the stomach and intestine. Control of cell-wall permeability is important to ensure that infiltration by the injectable aqueous carrier phase is absent or slow enough not to impair the echographic measurements but, in cases, still substantial to ensure relatively fast after-test biodegradability, i.e., ready metabolization of the suspension by the organism. Also the microporous structure of the microballoons envelope (pores of a few nm to a few hundreds of nm or more for microballoons envelopes of thickness ranging from 50-500 nm) is a factor of resiliency, i.e., the microspheres can readily accept pressure variations without breaking. The preferred range of pore sizes is about 50-2000 nm.

The conditions for achieving these results are met by using the method including the steps of (1) emulsifying a hydrophobic organic phase into a water phase so as to obtain droplets of the hydrophobic phase as an oil-in-water emulsion in the water phase; (2) adding to said emulsion a solution of at least one polymer in a volatile solvent insoluble in the water phase, so that a layer of said polymer will form around said droplets; then (3) evaporating the volatile solvent so that the polymer will deposit by interfacial precipitation around the droplets which then form beads with a core of the hydrophobic phase encapsulated by a membrane of the polymer, the beads being in suspension in the water phase; and finally (4) subjecting the suspension to reduced pressure under conditions such that the encapsulated hydrophobic phase can be removed by evaporation.

The hydrophobic phase selected in step (4) so it evaporates substantially simultaneously with the water phase and is replaced by air or gas, whereby dry, free flowing, readily dispersible microballoons are obtained.

One factor which enables to control the permeability of the microballoons membrane is the rate of evaporation of the hydrophobic phase relative to that of water in step (4) of the method, e.g., under conditions of freeze drying. For instance if the evaporation in is carried out between about −40° and 0° C., and hexane is used as the hydrophobic phase, polystyrene being the interfacially deposited polymer, beads with relatively large pores are obtained; this is so because the vapour pressure of the hydrocarbon in the chosen temperature range is significantly greater than that of water, which means that the pressure difference between the inside and outside of the spheres will tend to increase the size of the pores in the spheres membrane through which the inside material will be evaporated. In contrast, using cyclooctane as the hydrophobic phase (at −17° C. the vapour pressure is the same as that of water) will provide beads with very tiny pores because the difference of pressures between the inside and outside of the spheres during evaporation is minimized.

Depending on degree of porosity the microballoons of this invention can be made stable in an aqueous carrier from several hours to several months and give reproducible echographic signals for a long period of time. Actually, depending on the polymer selected, the membrane of the microballoons can be made substantially impervious when suspended in carrier liquids of appropriate osmotic properties, i.e., containing solutes in appropriate concentrations. It should be noted that the existence of micropores in the envelope of the microballoons of the present invention appears to be also related with the echographic response, i.e.,, all other factors being constant, microporous vesicles provide more efficient echographic signal than corresponding non-porous vesicles. The reason is not known but it can be postulated that when a gas is in resonance in a closed structure, the damping properties of the latter may be different if it is porous or non-porous.

Other non water soluble organic solvents which have a vapour pressure of the same order of magnitude between about −40° C. and 0° C. are convenient as hydrophobic solvents in this invention. These include hydrocarbons such as for instance n-octane, cyclooctane, the dimethylcyclohexanes, ethyl-cyclohexane, 2-, 3- and 4-methyl-heptane, 3-ethyl-hexane, toluene, xylene, 2-methyl-2-heptane, 2,2,3,3-tetramethylbutane and the like. Esters such as propyl and isopropyl butyrate and isobutyrate, butyl-formate and the like, are also convenient in this range. Another advantage of freeze drying is to operate under reduced pressure of a gas instead of air, whereby gas filled microballoons will result. Physiologically acceptable gases such as CO₂, N₂O, methane, Freon, helium and other rare gases are possible. Gases with radioactive tracer activity can be contemplated.

As the volatile solvent insoluble in water to be used for dissolving the polymer to be precipitated interfacially, one can cite halo-compounds such as CCl₄, CH₃Br, CH₂Cl₂, chloroform, Freon, low boiling esters such as methyl, ethyl and propyl acetate as well as lower ethers and ketones of low water solubility. When solvents not totally insoluble in water are used, e.g., diethyl-ether, it is advantageous to use, as the aqueous phase, a water solution saturated with said solvent beforehand.

The aqueous phase in which the hydrophobic phase is emulsified as an oil-in-water emulsion preferably contains 1-20% by weight of water-soluble hydrophilic compounds like sugars and polymers as stabilizers, e.g., polyvinyl alcohol (PVA), polyvinyl pyrrolidone (PVP), polyethylene glycol (PEG), gelatin, polyglutamic acid, albumin, and polysaccharides such as starch, dextran, agar, xanthan and the like. Similar aqueous phases can be used as the carrier liquid in which the microballoons are suspended before use.

Part of this water-soluble polymer can remain in the envelope of the microballoons or it can be removed by washing the beads before subjecting them to final evaporation of the encapsulated hydrophobic core phase.

The emulsifiers to be used (0.1-5% by weight) to provide the oil-in-water emulsion of the hydrophobic phase in the aqueous phase include most physiologically acceptable emulsifiers, for instance egg lecithin or soya bean lecithin, or synthetic lecithins such as saturated synthetic lecithins, for example, dimyristoyl phosphatidyl choline, dipalmitoyl phosphatidyl choline or distearoyl phosphatidyl choline or unsaturated synthetic lecithins, such as dioleyl phosphatidyl choline or dilinoleyl phosphatidyl choline. Emulsifiers also include surfactants such as free fatty acids, esters of fatty acids with polyoxyalkylene compounds like polyoxypropylene glycol and polyoxyethylene glycol; ethers of fatty alcohols with polyoxyalkylene glycols; esters of fatty acids with polyoxyalkylated sorbitan; soaps: glycerol-polyalkylene stearate; glycerol-polyoxyethylene ricinoleate; homo- and copolymers of polyalkylene glycols; polyethoxylated soya-oil and castor oil as well as hydrogenated derivatives; ethers and esters of sucrose or other carbohydrates with fatty acids, fatty alcohols, these being optionally polyoxyalkylated; mono-, di- and triglycerides of saturated or unsaturated fatty acids; glycerides or soya-oil and sucrose.

The polymer which constitutes the envelope or bounding membrane of the injectable microballoons can be selected from most hydrophilic, biodegradable physiologically compatible polymers. Among such polymers one can cite polysaccharides of low water solubility, polylactides and polyglycolides and their copolymers, copolymers of lactides and lactones such as ε-caprolactone, δ-valerolactone, polypeptides, and proteins such as gelatin, collagen, globulins and albumins. The great versatility in the selection of synthetic polymers is another advantage of the present invention since, as with allergic patients, one may wish to avoid using microballoons made of natural proteins (albumin, gelatin) like in U.S. Pat. No. 4,276,885 or EP-A-324,938. Other suitable polymers include poly-(ortho)esters (see for instance U.S. Pat. No. 4,093,709; U.S. Pat. No. 4,131,648; U.S. Pat. No. 4,138,344; U.S. Pat. No. 4,180,646); polylactic and polyglycolic acid and their copolymers, for instance DEXON (see J. Heller, Biomaterials 1 (1980), 51; poly(DL-lactide-co-δ-caprolactone), poly(DL-lactide-co-δ-valerolactone), poly(DL-lactide-co-δ-butyrolactone), polyalkylcyanoacrylates; polyamides, polyhydroxybutyrate; polydioxanone; poly-β-aminoketones (Polymer 23 (1982), 1693); polyphosphazenes (Science 193 (1976), 1214); and polyanhydrides. References on biodegradable polymers can be found in R. Langer et al., Macromol. Chem. Phys. C23 (1983), 61-126. Polyamino-acids such as polyglutamic and polyaspartic acids can also be used as well as their derivatives, i.e., partial esters with lower alcohols or glycols. One useful example of such polymers is poly-(t.butyl-glutamate). Copolymers with other amino-acids such as methionine, leucine, valine, proline, glycine, alamine, etc. are also possible. Recently some novel derivatives of polyglutamic and polyaspartic acid with controlled biodegradability have been reported (see W087/03891; U.S. Pat. No. 4,888,398 and EP-130.935 incorporated here by reference). These polymers (and copolymers with other amino-acids) have formulae of the following type:
—(NH—CHA-CO)ₓ(NH—CHX—CO)_{y}
where X designates the side chain of an amino-acid residue and A is a group of formula —(CH₂)ₙCOOR¹R²—OCOR (II), with R¹ and R² being H or lower alkyls, and R being alkyl or aryl; or R and R¹ are connected together by a substituted or unsubstituted linking member to provide 5- or 6-membered rings.

A can also represent groups of formulae:
—(CH₂)ₙCOO—CHR¹COOR (I)
and
—(CH₂)ₙCO(NH—CHX—CO)ₘNH—CH(COOH)—(CH₂)ₚCOOH (III)
and corresponding anhydrides. In all these formulae n, m and p are lower integers (not exceeding 5) and x and y are also integers selected for having molecular weights not below 5000.

The aforementioned polymers are suitable for making the microballoons according to the invention and, depending on the nature of substituents R, R¹, R² and X, the properties of the membrane can be controlled, for instance, strength, elasticity and biodegradability. For instance X can be methyl(alanine), isopropyl(valine), isobutyl(leucine and isoleucine), benzyl(phenylalanine).

Additives can be incorporated into the polymer wall of the microballoons to modify the physical properties such as dispersibility, elasticity and water permeability. For incorporation in the polymer, the additives can be dissolved in the polymer carrying phase, e.g., the hydrophobic phase to be emulsified in the water phase, whereby they will co-precipitate with the polymer during inter-facial membrane formation.

Among the useful additives, one may cite compounds which can “hydrophobize” the microballoons membrane in order to decrease water permeability, such as fats, waxes and high molecular-weight hydrocarbons. Additives which improve dispersibility of the microballoons in the injectable liquid-carrier are amphipatic compounds like the phospholipids; they also increase water permeability and rate of biodegradability.

Non-biodegradable polymers for making microballoons to be used in the digestive tract can be selected from most water-insoluble, physiologically acceptable, bioresistant polymers including polyolefins(polystyrene), acrylic resins(polyacrylates, polyacrylonitrile), polyesters(polycarbonate), polyurethanes, polyurea and their copolymers. ABS (acryl-butadienestyrene) is a preferred copolymer.

Additives which increase membrane elasticity are the plasticisers like isopropyl myristate and the like. Also, very useful additives are constituted by polymers akin to that of the membrane itself but with relatively low molecular weight. For instance when using copolymers of polylactic/polyglycolic type as the membrane forming material, the properties of the membrane can be modified advantageously (enhanced softness and biodegradability) by incorporating, as additives, low molecular weight (1,000 to 15,000 Dalton) polyglycolides or polylactides. Also polyethylene glycol of moderate to low M[w](e.g., PEG 2000) is a useful softening additive.

Preferably the plasticizers include isopropyl myristate, glyceryl monostearate and the like to control flexibility, the amphipatic substances include surfactants and phospholipids like the lecithins to control permeability by increasing porosity while the hydrophobic compounds include high molecular weight hydrocarbon like the paraffin-waxes to reduce porosity.

The quantity of additives to be incorporated in the polymer forming the inter-facially deposited membrane of the present microballoons is extremely variable and depends on the needs. In some cases no additive is used at all; in other cases amounts of additives which may reach about 20% by weight of the polymer are possible.

The injectable microballoons of the present invention can be stored dry in the presence or in the absence of additives to improve conservation and prevent coalescence. As additives, one may select from 0.1 to 25% by weight of water-soluble physiologically acceptable compounds such as mannitol, galactose, lactose or sucrose or hydrophilic polymers like dextran, xanthan, agar, starch, PVP, polyglutamic acid, polyvinylalcohol (PVA), albumin and gelatin. The useful life-time of the microballoons in the injectable liquid carrier phase, i.e., the period during which useful echographic signals are observed, can be controlled to last from a few minutes to several months depending on the needs; this can be done by controlling the porosity of the membrane from substantial imperviousness toward carrier liquids to porosities having pores of a few nanometers to several hundreds of nanometers. This degree of porosity can be controlled, in addition to properly selecting the membrane forming polymer and polymer additives, by adjusting the evaporation rate and temperature in step (4) of the method and properly selecting the nature of the compound (or mixture of compounds) constituting the hydrophobic phase, i.e., the greater the differences in its partial pressure of evaporation with that of the water phase, the coarser the pores in the microballoons membrane will be. Of course, this control by selection of the hydrophobic phase can be further refined by the choice of stabilizers and by adjusting the concentration thereof in order to control the rate of water evaporation during the forming of the microballoons. All these changes can easily be made by skilled persons without exercizing inventiveness and need not be further discussed.

It should be remarked that although the microballoons of this invention can be marketed in the dry state, more particularly when they are designed with a limited life time after injection, it may be desirable to also sell ready preparations, i.e., suspensions of microballoons in an aqueous liquid carrier ready for injection or oral administration. This requires that the membrane of the microballoons be substantially impervious (at least for several months or more) to the carrier liquid. It has been shown in this description that such conditions can be easily achieved with the present method by properly selecting the nature of the polymer and the interfacial deposition parameters. Actually parameters have been found (for instance using the polyglutamic polymer (where A is the group of formula II) and cyclooctane as the hydrophobic phase) such that the porosity of the membrane after evaporation of the hydrophobic phase is so tenuous that the microballoons are substantially impervious to the aqueous carrier liquid in which they are suspended.

A preferred administrable preparation for diagnostic purposes comprises a suspension in buffered or unbuffered saline (0.9% aqueous NaCl; buffer 10 mM tris-HCl) containing 10⁸-10¹⁰ vesicles/ml. This can be prepared mainly according to the directions of the Examples below, preferably Examples 3 and 4, using poly-(DL-lactide) polymers from the Company Boehringer, Ingelheim, Germany.

The invention described up until this point can be further elucidated by the description of the following representative (but not limiting) embodiments, numbered 1-31:

1. Microcapsules or microballoons of micronic or submicronic size bounded by a polymer membrane filled with air or a gas suitable, when in the form of suspensions in a liquid carrier, to be administered to human or animal patients for therapeutic or diagnostic applications, e.g., for the purpose of ultrasonic echography imaging, characterized in that the polymer of the membrane is a deformable and resilient interfacially deposited polymer.

2. Air or gas filled microballoons bounded by an elastic interfacial polymeric membrane adapted to form with suitable physiologically acceptable aqueous carrier liquids suspensions to be taken orally, rectally and urethrally, or injectable into living organisms for therapeutic or diagnostic purposes, characterized in being non-coalescent dry and instantly dispersible by admixing with said liquid carrier.

3. The microballoons of embodiments 1 or 2 having size mostly in the 0.5-10 μm range suitable for injection into the bloodstream of living beings, characterized in that the membrane polymer is biodegradable and the membrane is either impervious or contains pores permeable to bioactive liquids for increasing the rate of biodegradation.

4. The microballoons of embodiment 3, in which the polymer membrane has a porosity ranging from a few nanometers to several hundreds or thousands of nanometers, preferable 50-2000 nm.

5. The microballoons of embodiment 3, in which the membrane is elastic, has a thickness of 50-500 nm, and resists pressure variations produced by heart beat pulsations in the bloodstream.

6. The microballoons of embodiment 3, in which the polymer of the membrane is a biodegradable polymer selected from polysaccharides, polyamino-acids, polylactides and polyglycolides and their copolymers, copolymers of lactides and lactones, polypeptides, poly-(ortho)esters, polydioxanone, poly-β-aminoketones, polyphosphazenes, polyanhydrides and poly(alkyl-cyano-acrylates).

7. The microballoons of embodiment 3, in which the membrane polymer is selected from polyglutamic or polyaspartic acid derivatives and their copolymers with other amino-acids.

8. The microballoons of embodiment 7, in which the polyglutamic and polyaspartic acid derivatives are selected from esters and amides involving the carboxylated side function thereof, said side functions having formulae
—(CH₂)ₙCOO—CHR¹COOR (I), or
—(CH₂)ₙCOOR¹R²—O—COR (II), or
—(CH₂)ₙCO(NH—CHX—CO)ₘNHCH(COOH)—(CH₂)ₚCOOH (III),
in which R is an alkyl or aryl substituent; R¹ and R² are H or lower alkyls, or R and R¹ are connected together by a substituted or unsubstituted linking member to form a 5- or 6-membered ring; n is 1 or 2; p is 1, 2 or 3; m is an integer from 1 to 5 and X is a side chain of an aminoacid residue.

9. The microballoons of embodiment 3, in which the membrane polymer contains additives to control the degree of elasticity, and the size and density of the pores for permeability control.

10. The microballoons of embodiment 9, in which said additives include plasticizers, amphipatic substances and hydrophobic compounds.

11. The microballoons of embodiment 10, in which the plasticizers include isopropyl myristate, glyceryl monostearate and the like to control flexibility, the amphipatic substances include surfactants and phospholipids like the lecithins to control permeability by increasing porosity and the hydrophobic compounds include high molecular weight hydrocarbon like the paraffin-waxes to reduce porosity.

12. The microballoons of embodiment 10, in which the additives include polymers of low molecular weight, e.g., in the range of 1,000 to 15,000, to control softness and resiliency of the microballoon membrane.

13. The microballoons of embodiment 12, in which the low molecular weight polymer additives are selected from polylactides, polyglycolides, polyalkylene glycols like polyethylene glycol and polypropylene glycol, and polyols like polyglycerol.

14. The microballoons of embodiments 1 or 2, having size up to about 1000 μm suitable for oral, rectal and urethral applications, characterized in that the membrane polymer is not biodegradable in the digestive tract and impervious to biological liquids.

15. The microballoons of embodiment 14, in which the polymer is selected from polylefins, polyacrylates, polyacrylonitrile, non-hydrolyzable polymesters, polyurethanes and polyureas.

16. Aqueous suspension of the microballoons according to embodiments 1 or 2 for administration to patients, characterized in containing a concentration of about 10⁶ to 10¹⁰ microballoons/ml, this being stable for period exceeding a month.

17. A method for making air or gas filled microballoons usable as suspensions in a carrier liquid for oral, rectal and urethral applications, or for injections into living organisms, this method comprising the steps of:

(a) emulsifying a hydrophobic organic phase into a water phase so as to obtain droplets of said hydrophobic phase as an oil-in-water emulsion in said water phase;

(b) adding to said emulsion a solution of at least one polymer in a volatile solvent insoluble in the water phase, so that a later of said polymer will form around said droplets;

(c) evaporating said volatile solvent so that the polymer will deposit by interfacial precipitation around the droplets which then form beads with a core of said hydrophobic phase encapsulated by a membrane of said polymer, said beads being in suspension in said water phase;

(d) subjecting said suspension to reduced pressure under conditions such that said encapsulated hydrophobic phase be removed by evaporation;

characterized in that said hydrophobic phase is selected so that in step (4) it evaporates substantially simultaneously with the water phase and is replaced by air or gas, whereby dry, free flowing, readily dispersible microballoons are obtained.

18. The method of embodiment 17, in which said polymer is dissolved in said hydrophobic phase, so that steps (2) and (3) can be omitted and the polymer membrane will form by interfacial precipitation during step (4).

19. The method of embodiment 17, characterized in that evaporation of said hydrophobic phase in step (4) is performed at a temperature where the partial vapour pressure of said hydrophobic phase is of the same order as that of water vapour.

20. The method of embodiment 17, in which said evaporation of step (4) is carried out under freeze-drying conditions.

21. The method of embodiment 20, in which freeze-drying is effected at temperatures of from −40° C. to 0° C.

22. The method of embodiments 17 or 19, in which the hydrophobic phase is selected from organic compounds having a vapour pressure of about 1 Torr at a temperature comprised in the interval of about −40° C. to 0° C.

23. The method of embodiments 17 or 18, in which the aqueous phase comprises, dissolved, from about 1 to 20% by weight of stabilizers comprising hydrophilic compound selected from sugars, PVA, PVP, gelatin, starch, dextran, polydextrose, albumin and the like.

24. The method of embodiment 18, in which additives to control the degree of permeability of the microballoons membrane are added to the hydrophobic phase, the rate of biodegradability of the polymer after injecting the microballoons into living organisms being a function of said degree of permeability.

25. The method of embodiment 24, in which the said additives include hydrophobic solids like fats, waxes and high molecular weight hydrocarbons, the presence of which in the membrane polymer of the microballoons will reduce permeability toward aqueous liquids.

26. The method of embodiment 24, in which the said additives include amphipatic compounds like the phospholids, or low molecular weight polymers, the presence of which in the membrane polymer will increase permeability of the microballoons to aqueous liquids.

27. The method of embodiment 18, in which the hydrophobic phase subjected to emulsification in said water phase also contains a water-soluble solvent which, upon being diluted into said water phase during emulsification, will aid in reducing the size of droplets and induce interfacial precipitation of the polymer before step (4) is carried out.

28. A method for making air or gas filled microballoons usable as suspensions in a carrier liquid for oral, rectal and urethral applications, or for injections into living organisms, this method comprising the steps of,

(a) emulsifying a hydrophobic organic phase into a water phase so as to obtain droplets of said hydrophobic organic phase as an oil-in-water emulsion in said water phase, said organic phase containing, dissolved therein, one or more water-insoluble polymers;

(b) subjecting said emulsion to reduced pressure under conditions such that said hydrophobic phase by removed by evaporation, whereby the polymer dissolved in the droplets will deposit interfacially and form a polymer bounding membrane, the droplets being simultaneously converted to microballoons,

characterized in that said hydrophobic phase is selected so that in step (2) it evaporates substantially simultaneously with the water phase and, upon evaporation, is replaced by air or gas, whereby the microballoons obtained are in dry, free flowing and readily dispersible form.

29. The method of embodiment 28, in which the hydrophobic polymer solution phase subjected to emulsification in said water phase also contains a water-soluble solvent which, upon being diluted into said water phase during emulsification, will aid in reducing the size of droplets and induce interfacial precipitations of the polymer before step (2) is carried out.

30. The method of embodiment 28, in which said organic hydrophobic phase emulsified in step (1) contains no polymer dissolved therein, and before carrying through step (2), the following additional steps are performed:

(a) adding to said emulsion a solution of at least one polymer in a volatile solvent insoluble in the water phase, so that a layer of said polymer will form around said droplets;

(b) evaporating said volatile solvent so that the polymer will deposit by interfacial precipitation around the droplets, thus forming microballoons or beads with a core of said hydrophobic phase encapsulated by a membrane of said polymer, said beads being in suspension in said water phase, whereby in step (2) evaporation of said hydrophobic phase takes place through said membrane and provides it with substantial microporosity.

31. An injectable aqueous suspension of microballoons containing 10⁸-10¹⁰ vesicles/ml bounded by a membrane of interfacially precipitated DL-lactide polymer defined by the commercial name of Resomer.

The following Examples illustrate the invention practically:

### EXAMPLE 12

One gram of polystyrene was dissolved in 19 g of liquid naphthalene at 100° C. This naphthalene solution was emulsified at 90°-95° C. into 200 ml of a water solution of polyvinyl alcohol (PVA) (4% by weight) containing 0.1% of Tween-40 emulsifier. The emulsifying head was a Polytron PT-3000 at about 10,000 rpm. Then the emulsion was diluted under agitation with 500 ml of the same aqueous phase at 15° C. whereby the naphthalene droplets solidified into beads of less than 50 microns as ascertained by passing through a 50 micron mesh screen. The suspension was centrifugated under 1000 g and the beads washed with water and recentrifugated. This step was repeated twice.

The beads were resuspended in 100 ml of water with 0.8 g of dissolved lactose and the suspension was frozen into a block at −30° C. The block was thereafter evaporated under about 0.5-2 Torr between about −20° and −10° C. Air filled microballoons of average size 5-10 microns and controlled porosity were thus obtained which gave an echographic signal at 2.25 and 7.5 MHz after being dispersed in water (3% dispersion by weight). The stability of the microballoons in the dry state was effective for an indefinite period of time; once suspended in an aqueous carrier liquid the useful life-time for echography was about 30 min or more. Polystyrene being non-biodegradable, this material was not favored for injection echography but was useful for digestive tract investigations. This Example clearly establishes the feasibility of the method of the invention.

### EXAMPLE 13

A 50:50 copolymer mixture (0.3 g) of DL-lactide and glycolide (Du Pont Medisorb) and 16 mg of egg-lecithin were dissolved in 7.5 ml of CHCl₃ to give solution (1).

A solution (2) containing 20 mg of paraffin-wax (M.P. 54°-56° C.) in 10 ml of cyclooctane (M.P. 10-13°) was prepared and emulsified in 150 ml of a water solution (0.13% by weight) of 0.13% by weight) of Pluronic F-108 (a block copolymer of ethylene oxide and propylene oxide) containing also 1.2 g of CHCl₃. Emulsification was carried out at room temperature for 1 min with a Polytron head at 7000 rpm. Then solution (1) was added under agitation (7000 rpm) and, after about 30-60 sec, the emulsifier head was replaced by a helical agitator (500 rpm) and stirring was continued for about 3 hrs at room temperature (22° C.). The suspension was passed through a 50 micron screen and frozen to a block which was subsequently evaporated between −20° and 0° C. under high-vacuum (catching trap −60° to −80° C.). There were thus obtained 0.264 g (88%) of air-filled microballoons stable in the dry state.

Suspensions of said microballoons in water (no stabilizers) gave a strong echographic signal for at least one hour. After injection in the organism, they biodegraded in a few days.

### EXAMPLE 14

A solution was made using 200 ml of tetrahydrofuran (THF), 0.8 g of a 50:50 DL-lactide/glycolide copolymer (Boehringer AG), 80 mg of egg-lecithin, 64 mg of paraffin-wax and 4 ml of octane. This solution was emulsified by adding slowly into 400 ml of a 0.1% aqueous solution of Pluronic F-108 under helical agitation (500 r.p.m.). After stirring for 15 min, the milky dispersion was evaporated under 10-12 Torr 25° C. in a rotavapor until its volume was reduced to about 400 ml. The dispersion was sieved on a 50 micron grating, then it was frozen to −40° C. and freeze-dried under about 1 Torr. The residue, 1.32 g of very fine powder, was taken with 40 ml of distilled water which provided, after 3 min of manual agitation, a very homogeneous dispersion of microballoons of average size 4.5 microns as measured using a particle analyzer (Mastersizer from Malvern). The concentration of microballoons (Coulter Counter) was about 2×10⁹/ml. This suspension gave strong echographic signals which persisted for about 1 hr.

If in the present example, the additives to the membrane polymer are omitted, i.e., there is used only 800 mg of the lactide/glycolide copolymer in the THF/octane solution, a dramatic decrease in cell-wall permeability is observed, the echographic signal of the dispersion in the aqueous carrier not being significantly attenuated after 3 days.

Using intermediate quantities of additives provided beads with controlled intermediate porosity and life-time.

### EXAMPLE 15

There was used in this Example a polymer of formula defined in embodiment 1 in which the side group has formula (II) where R¹ and R² are hydrogen and R is tert.butyl. The preparation of this polymer (defined as poly-POMEG) is described in U.S. Pat. No. 4,888,398.

The procedure was like in Example 14, using 0.1 g poly-POMEG, 70 ml of THF, 1 ml of cyclooctane and 100 ml of a 0.1% aqueous solution of Pluronic F-108. No lecithin or high-molecular weight hydrocarbon was added. The milky emulsion was evaporated at 27° C./10 Torr until the residue was about 100 ml, then it was screened on a 50 micron mesh and frozen. Evaporation of the frozen block was carried out (0.5-1 Torr) until dry. The yield was 0.18 g because of the presence of the surfactant. This was dispersed in 10 ml of distilled water and counted with a Coulter Counter. The measured concentration was found to be 1.43×10⁹ microcapsules/ml, average size 5.21 microns as determined with a particle analyzer (Mastersizer from Malvern). The dispersion was diluted 100×, i.e., to give about 1.5×10⁷ microspheres/ml and measured for echogenicity. The amplitude of the echo signal was 5 times greater at 7.5 MHz than at 2.25 MHz. These signals were reproducible for a long period of time.

Echogenicity measurements were performed with a pulse-echo system consisting of a plexiglas specimen holder (diameter 30 mm) with a 20 micron thick Mylar acoustic window, a transducer holder immersed in a constant temperature water bath, a pulser-receiver (Accutron M3010JS) with an external pre-amplifier with a fixed gain of 40 dB and an internal amplifier with gain adjustable from −40 to +40 dB and interchangeable 13 mm unfocused transducers. A 10 MHz low-pass filter was inserted in the receiving part to improve the signal to noise ratio. The A/D board in the IBM PC was a Sonotek STH 832. Measurements were carried out at 2.25, 3.5, 5 and 7.5 MHz.

If in the present Example, the polymer used is replaced by lactic-lactone copolymers, the lactones being δ-butyrolactone, δ-valerolactone or ε-caprolactone (see Fukuzaki et al., J. Biomedical Mater. Res. 25 (1991), 315-328), similar favorable results were obtained. Also in a similar context, polyalkylcyano-acrylates and particularly a 90:10 copolymer poly(DL-lactide-co-glycolide) gave satisfactory results. Finally, a preferred polymer is a poly(DL-lactide) from the Company Boehringer-Ingelheim sold under the name “Resomer R-206” or Resomer R-207.

### EXAMPLE 16

Two-dimensional echocardiography was performed using an Acuson-128 apparatus with the preparation of Example 15 (1.43×10⁹/ml) in an experimental dog following peripheral vein injection of 0.1-2 ml of the dispersion. After normally expected contrast enhancement imaging of the right heart, intense and persistent signal enhancement of the left heart with clear outlining of the endocardium was observed, thereby confirming that the microballoons made with poly-POMEG (or at least a significant part of them) were able to cross the pulmonary capillary circulation and to remain in the blood-stream for a time sufficient to perform efficient echographic analysis.

In another series of experiments, persistent enhancement of the Doppler signal from systemic arteries and the portal vein was observed in the rabbit and in the rat following peripheral vein injection of 0.5-2 ml of a preparation of microballoons prepared as disclosed in Example 15 but using poly(DL-lactic acid) as the polymer phase. The composition used contained 1.9×10⁸ vesicles/ml.

Another composition prepared also according to the directions of Example 15 was achieved using poly(tert.butylglutamate). This composition (0.5 ml) at dilution of 3.4×10⁸ microballoons/ml was injected in the portal vein of rats and gave persistent contrast enhancement of the liver parenchyma.

### EXAMPLE 17

A microballoon suspension (1.1×10⁹ vesicles/ml) was prepared as disclosed in Example 12 (resin=polystyrene). One ml of this suspension was diluted with 100 ml of 300 mM mannitol solution and 7 ml of the resulting dilution was administered intragastrically to a laboratory rat. The animal was examined with an Acuson-128 apparatus for 2-dimensional echography imaging of the digestive tract which clearly showed the single loops of the small intestine and of the colon.

Further Methods of the Invention and Gases Used Therein

Despite the many progresses achieved regarding the stability under storage of aqueous microbubble suspensions, this being either in the precursor or final preparation stage, there still remained until now the problem of vesicle durability when the suspensions are exposed to overpressure, e.g., pressure variations such as that occurring after injection in the blood stream of a patient and consecutive to heart pulses, particularly in the left ventricle. Actually, the present inventors have observed that, for instance in anaesthetized rabbits, the pressure variations are not sufficient to substantially alter the bubble count for a period of time after injection. In contrast, in dogs and human patients, typical microbubbles or microballoons filled with common gases such as air, methane or CO₂ will collapse completely in a matter of seconds after injection due to the blood pressure effect. It became hence important to solve the problem and to increase the useful life of suspensions of microbubbles and membrane bounded microballoons under pressure in order to ensure that echographic measurements can be performed in vivo safely and reproducibly.

It should be mentioned at this stage that another category of echogenic image enhancing agents has been proposed which resist overpressures as they consist of plain microspheres with a porous structure, such porosity containing air or a gas. Such microspheres are disclosed for instance in WO-A-91/12823 (Delta Biotechnology), EP-A-0327490 (Schering) and EP-A-0458079 (Hoechst). The drawback with the plain porous microspheres is that the encapsulated gas-filled free space is generally too small for good echogenic response and the spheres lack adequate elasticity. Hence the preference generally remains with the hollow microvesicles and a solution to the collapsing problem was searched.

This problem has now been solved by using gases or gas mixtures in conformity with the criteria outlined in the embodiments shown below. Briefly, it has been found that when the echogenic microvesicles are made in the presence of a gas, respectively are filled at least in part with a gas, having physical properties in conformity with the equation below, then the microvesicles remarkably resist pressure >60 Torr after injection for a time sufficient to obtain reproducible echographic measurements:

$$\frac{{S}_{gas}}{{S}_{air}} \times \frac{{Mw}_{air}^{0.5}}{{Mw}_{gas}^{0.5}} ≦ 1$$

In the foregoing equation, “S” designates the solubilities in water expressed as the “Bunsen” coefficients, i.e., as volume of gas dissolved by unit volume of water under standard conditions (1 bar, 25° C.), and under partial pressure of the given gas of 1 atm (see the Gas Encyclopaedia, Elsevier 1976). Since, under such conditions and definitions, the solubility of air is 0.0167, and the square root of its average molecular weight (Mw) is 5.39, the above relation simplifies to:

$$\frac{{S}_{gas}}{{Mw}_{gas}^{0.5}} ≦ 0.0031$$

In the Examples to be found hereafter there is disclosed the testing of echogenic microbubbles and microballoons (see the Tables) filled with a number of different gases and mixtures thereof, and the corresponding resistance thereof to pressure increases, both in vivo and in vitro. In the Tables, the water solubility factors have also been taken from the aforecited Gas Encyclopaedia from “L'Air Liquide”, Elsevier Publisher (1976).

The microvesicles in aqueous suspension containing gases according to the invention include most microbubbles and microballoons disclosed until now for use as contrast agents for echography. The preferred microballoons are those disclosed herein (e.g., supra) and in EP-A-0324938, PCT/EP91/01706 and EP-A-0458745; the preferred microbubbles are those of the compositions disclosed herein (e.g., supra) and in PCT/EP91/00620; these microbubbles are advantageously formed from an aqueous liquid and a dry powder (microvesicle precursors) containing lamellarized freeze-dried phospholipids and stabilizers; the microbubbles are developed by agitation of this powder in admixture with the aqueous liquid carrier. The microballoons of EP-A-0458745 have a resilient interfacially precipitated polymer membrane of controlled porosity. They are generally obtained from emulsions into microdroplets of polymer solutions in aqueous liquids, the polymer being subsequently caused to precipitate from its solution to form a fibrogenic membrane at the droplet/liquid interface, which process leads to the initial formation of liquid-filled microvesicles, the liquid core thereof being eventually substituted by a gas.

In order to carry out the method of the present invention, i.e., to form or fill the microvesicles, whose suspensions in aqueous carriers constitute the desired echogenic additives, with the gases according to the foregoing relation, one can either use, as a first embodiment, a two step route consisting of (1) making the microvesicles from appropriate starting materials by any suitable conventional technique in the presence of any suitable gas, and (2) replacing this gas originally used (first gas) for preparing the microvesicles with a new gas (second gas) according to the invention (gas exchange technique).

Otherwise, according to a second embodiment, one can directly prepare the desired suspensions by suitable usual methods under an atmosphere of the new gas according to the invention.

If one uses the two-step route, the initial gas can be first removed from the vesicles (for instance by evacuation under suction) and thereafter replaced by bringing the second gas into contact with the evacuated product, or alternatively, the vesicles still containing the first gas can be contacted with the second gas under conditions where the second gas will displace the first gas from the vesicles (gas substitution). For instance, the vesicle suspensions, or preferably precursors thereof (precursors here may mean the materials the microvesicle envelopes are made of, or the materials which, upon agitation with an aqueous carrier liquid, will generate or develop the formation of microbubbles in this liquid), can be exposed to reduced pressure to evacuate the gas to be removed and then the ambient pressure is restored with the desired gas for substitution. This step can be repeated once or more times to ensure complete replacement of the original gas by the new one. This embodiment applies particularly well to precursor preparations stored dry, e.g., dry powders which will regenerate or develop the bubbles of the echogenic additive upon admixing with an amount of carrier liquid. Hence, in one preferred case where microbubbles are to be formed from an aqueous phase and dry laminarized phospholipids, e.g., powders of dehydrated lyophilized liposomes plus stabilizers, which powders are to be subsequently dispersed under agitation in a liquid aqueous carrier phase, it is advantageous to store this dry powder under an atmosphere of a gas selected according to the invention. A preparation of such kind will keep indefinitely in this state and can be used at any time for diagnosis, provided it is dispersed into sterile water before injection.

Otherwise, and this is particularly so when the gas exchange is applied to a suspension of microvesicles in a liquid carrier phase, the latter is flushed with the second gas until the replacement (partial or complete) is sufficient for the desired purpose. Flushing can be effected by bubbling from a gas pipe or, in some cases, by simply sweeping the surface of the liquid containing the vesicles under gentle agitation with a stream (continuous or discontinuous) of the new gas. In this case, the replacement gas can be added only once in the flask containing the suspension and allowed to stand as such for a while, or it can be renewed one or more times in order to assure that the degree of renewal (gas exchange) is more or less complete.

Alternatively, in a second embodiment as said before, one will effect the full preparation of the suspension of the echogenic additives starting with the usual precursors thereof (starting materials), as recited in the prior art and operating according to usual means of said prior art, but in the presence of the desired gases or mixture of gases according to the invention instead of that of the prior art which usually recites gases such as air, nitrogen, CO₂ and the like.

It should be noted that in general the preparation mode involving one first type of gas for preparing the microvesicles and, thereafter, substituting the original gas by a second kind of gas, the latter being intended to confer different echogenic properties to said microvesicles, has the following advantage: As will be best seen from the results in the Examples hereinafter, the nature of the gas used for making the microvesicles, particularly the microballoons with a polymer envelope, has a definitive influence on the overall size (i.e., the average mean diameter) of said microvesicles; for instance, the size of microballoons prepared under air with precisely set conditions can be accurately controlled to fall within a desired range, e.g., the 1 to 10 μm range suitable for echographying the left and right heart ventricles. This not so easy with other gases, particularly the gases in conformity with the requirements of the present invention; hence, when one wishes to obtain microvesicles in a given size range but filled with gases the nature of which would render the direct preparation impossible or very hard, one will much advantageously rely on the two-steps preparation route, i.e., one will first prepare the microvesicles with a gas allowing more accurate diameter and count control, and thereafter replace the first gas by a second gas by gas exchange.

In the description of the Experimental part that follows (Examples), gas-filled microvesicles suspended in water or other aqueous solutions have been subjected to pressures over that of ambient. It was noted that when the overpressure reached a certain value (which is generally typical for a set of microsphere parameters and working conditions like temperature, compression rate, nature of carrier liquid and its content of dissolved gas (the relative importance of this parameter will be detailed hereinafter), nature of gas filler, type of echogenic material, etc.), the microvesicles started to collapse, the bubble count progressively decreasing with further increasing the pressure until a complete disappearance of the sound reflector effect occurred. This phenomenon was better followed optically, (nephelometric measurements) since it is paralleled by a corresponding change in optical density, i.e., the transparency of the medium increases as the bubble progressively collapse. For this, the aqueous suspension of microvesicles (or an appropriate dilution thereof was placed in a spectrophotometric cell maintained at 25° C. (standard conditions) and the absorbance was measured continuously at 600 or 700 nm, while a positive hydrostatic overpressure was applied and gradually increased. The pressure was generated by means of a peristaltic pump (Gilson's Mini-puls) feeding a variable height liquid column connected to the spectrophotometric cell, the latter being sealed leak-proof The pressure was measured with a mercury manometer calibrated in Torr. The compression rate with time was found to be linearly correlated with the pump's speed (rpm's). The absorbance in the foregoing range was found to be proportional to the microvesicle concentration in the carrier liquid.

The invention will now be further described with reference to FIG. 1 which is a graph which relates the bubble concentration (bubble count), expressed in terms of optical density in the aforementioned range, and the pressure applied over the bubble suspension. The data for preparing the graph are taken from the experiments reported in Example 21.

FIG. 1 shows graphically that the change of absorbance versus pressure is represented by a sigmoid-shaped curve. Up to a certain pressure value, the curve is nearly flat which indicates that the bubbles are stable. Then, a relatively fast absorbance drop occurs, which indicates the existence of a relatively narrow critical region within which any pressure increase has a rather dramatic effect on the bubble count. When all the microvesicles have disappeared, the curve levels off again. A critical point on this curve was selected in the middle between the higher and lower optical readings, i.e., intermediate between the “full”-bubble (OD max) and the “no”-bubble (OD min) measurements, this actually corresponding where about 50% of the bubbles initially present have disappeared, i.e., where the optical density reading is about half the initial reading, this being set, in the graph, relative to the height at which the transparency of the pressurized suspension is maximal (base line). This point which is also in the vicinity where the slope of the curve is maximal is defined as the critical pressure PC. It was found that for a given gas, PC does not only depend on the aforementioned parameters but also, and particularly so, on the actual concentration of gas (or gases) already dissolved in the carrier liquid: the higher the gas concentration, the higher the critical pressure. In this connection, one can therefore increase the resistance to collapse under pressure of the microvesicles by making the carrier phase saturated with a soluble gas, the latter being the same, or not, (i.e., a different gas) as the one that fills the vesicles. As an example, air-filled microvesicles could be made very resistant to overpressures (>120 Torr) by using, as a carrier liquid, a saturated solution of CO₂. Unfortunately, this finding is of limited value in the diagnostic field since once the contrast agent is injected to the bloodstream of patients (the gas content of which is of course outside control), it becomes diluted therein to such an extent that the effect of the gas originally dissolved in the injected sample becomes negligible.

Another readily accessible parameter to reproducibly compare the performance of various gases as microsphere fillers is the width of the pressure interval (ΔP) limited by the pressure values under which the bubble counts (as expressed by the optical densities) is equal to the 75% and 25% of the original bubble count. Now, it has been surprisingly found that for gases where the pressure difference ΔP=P₂₅−P₇₅ exceeds a value of about 25-30 Torr, the killing effect of the blood pressure on the gas-filled microvesicles is minimized, i.e., the actual decrease in the bubble count is sufficiently slow not to impair the significance, accuracy and reproducibility of echographic measurements.

It was found, in addition, that the values of PC and ΔP also depend on the rate of rising the pressure in the test experiments illustrated by FIG. 1, i.e., in a certain interval of pressure increase rates (e.g., in the range of several tens to several hundreds of Torr/min), the higher the rate, the larger the values for PC and ΔP. For this reason, the comparisons effected under standard temperature conditions were also carried out at the constant increase rate of 100 Torr/min. It should however be noted that this effect of the pressure increase rate on the measure of the PC and ΔP values levels off for very high rates; for instance the values measured under rates of several hundreds of Torr/min are not significantly different from those measured under conditions ruled by heart beats.

Although the very reasons why certain gases obey the aforementioned properties, while others do not, have not been entirely clarified, it would appear that some relation possibly exists in which, in addition to molecular weight and water solubility, dissolution kinetics, and perhaps other parameters, are involved. However these parameters need not be known to practice the present invention since gas eligibility can be easily determined according to the aforediscussed criteria.

The gaseous species which particularly suit the invention are, for instance, halogenated hydrocarbons like the freons and stable fluorinated chalcogenides like SF₆, SeF₆ and the like. Although in conjunction with suitable surfactants and stabilizers, the gases used may include gases like sulfur hexafluoride, tetrafluoromethane, chlorotrifluoromethane, dichlorodifluoro-methane, bromotrifluoromethane, bromochlorodifluoromethane, dibromo-difluoromethane dichlorotetrafluoroethane, chloropentafluoroethane, hexafluoroethane, hexafluoropropylene, octafluoropropane, hexafluoro-butadiene, octafluoro-2-butene, octafluorocyclobutane, decafluorobutane, perfluorocyclopentane, dodecafluoropentane and more preferably sulfur hexafluoride and/or octafluorocyclobutane, may be used. The media of the invention preferably contains a gas that includes one selected from sulfur hexafluoride, tetrafluoromethane, hexafluoroethane, hexafluoro-propylene, octafluoropropane, hexafluorobutadiene, octafluoro-2-butene, octafluorocyclobutane, decafluorobutane, perfluorocyclopentane, dodecafluoropentane and more preferably sulfur hexafluoride and/or octafluorocyclobutane.

It has been mentioned above that the degree of gas saturation of the liquid used as carrier for the microvesicles according to the invention has an importance on the vesicle stability under pressure variations. Indeed, when the carrier liquid in which the microvesicles are dispersed for making the echogenic suspensions of the invention is saturated at equilibrium with a gas, preferably the same gas with which the microvesicles are filled, the resistance of the microvesicles to collapse under variations of pressure is markedly increased. Thus, when the product to be used as a contrast agent is sold dry to be mixed just before use with the carrier liquid (see for instance the products disclosed in PCT/EP91/00620 mentioned hereinbefore), it is quite advantageous to use, for the dispersion, a gas saturated aqueous carrier. Alternatively, when marketing ready-to-use microvesicle suspensions as contrast agents for echography, one will advantageously use as the carrier liquid for the preparation a gas saturated aqueous solution; in this case the storage life of the suspension will be considerably increased and the product may be kept substantially unchanged (no substantial bubble count variation) for extended periods, for instance several weeks to several months, and even over a year in special cases. Saturation of the liquid with a gas may be effected most easily by simply bubbling the gas into the liquid for a period of time at room temperature.

The invention described herein can be further elucidated by the description of the following representative (but not limiting) embodiments, numbered 1-18:

1. A method for imparting resistance against collapsing to contrast agents for ultrasonic echography which consist of gas-filled microvesicles in suspension in aqueous liquid carrier phases, i.e., either microbubbles bounded by an evanescent gas/liquid interfacial closed surface, or microballoons bounded by a material envelope, said collapsing resulting, at least in part, from pressure increases effective, e.g., when the said suspensions are injected into the blood stream of patients, said method comprising forming said microvesicles in the presence of a gas, or if the microvesicles are already made filling them with this gas, which is a physiologically acceptable gas, or gas mixture, at least a fraction of which has a solubility in water expressed in liters of gas by liter of water under standard conditions divided by the square root of the molecular weight in daltons which does not exceed 0.003.

2. The method of embodiment 1, which is carried out in two steps, in the first step the microvesicles or dry precursors thereof are initially prepared under an atmosphere of a first gas, then in the second step at least a fraction of the first gas is substantially substituted by a second gas, the latter being said physiologically acceptable gas.

3. The method of embodiment 1, in which the physiologically acceptable gas used is selected from SF₆ or Freonig such as CF₄, CBrF₃, C₄F₈, CClF₃, CCl₂F₂, C₂F₆, C₃F₈, C₄F₆, C₅F₁₀, C₅F₁₂, C₂ClF₅, CBrClF₂, C₂Cl₂F₄, CBr₂F₂ and C₄F₁₀.

4. The method of embodiment 2, in which the gas used in the first step is a kind that allows effective control of the average size and concentration of the microvesicles in the carrier liquid, and the physiologically acceptable gas added in the second step ensures prolonged useful echogenic life to the suspension for in vivo ultrasonic imaging.

5. The method of embodiment 1, in which the aqueous phase carrying the microbubbles contains dissolved film-forming surfactants in lamellar or laminar form, said surfactants stabilizing the microbubbles boundary at the gas to liquid interface.

6. The method of embodiment 5, in which said surfactants comprise one or more phospholipids.

7. The method of embodiment 6, in which at least part of the phospholipids are in the form of liposomes.

8. The method of embodiment 6, in which at least one of the phospholipids is a diacylphosphatidyl compound wherein the acyl group is a C₁₆ fatty acid residue or a higher homologue thereof.

9. The method of embodiments 1 and 2, in which the microballoon material envelope is made of an organic polymeric membrane.

10. The method of embodiment 9, in which the polymers of the membrane are selected from polylactic or polyglycolic acid and their copolymers, reticulated serum albumin, reticulated haemoglobin, polystyrene, and esters of polyglutamic and polyaspartic acids.

11. The method of embodiment 1, in which the forming of the microvesicles with said physiologically acceptable gas is effected by alternately subjecting dry precursors thereof to reduced pressure and restoring the pressure with said gas, and finally dispersing the precursors in a liquid carrier.

12. The method of embodiment 1, in which the filling of the microballoons with said physiologically acceptable gas is effected by simply flushing the suspension with said gas under ambient pressure.

13. The method of embodiment 1, which comprises making the microvesicles by any standard method known in the art but operating under an atmosphere composed at least in part of said gas.

14. Suspensions of gas filled microvesicles distributed in an aqueous carrier liquid to be used as contrast agents in ultrasonic echography, characterized in that the gas is physiologically acceptable and such that at least a portion thereof has a solubility in water, expressed in liter of gas by liter of water under standard conditions, divided by the square root of the molecular weight which does not exceed 0.003.

15. The aqueous suspensions of embodiment 14, characterized in that the gas is such that the pressure difference ΔP between those pressures which, when applied under standard conditions and at a rate of about 100 Torr/min to the suspension cause the collapsing of about 75%, respectively 25%, of the microvesicles initially present, is at least 25 Torr.

16. Aqueous suspensions according to embodiment 14, in which the microvesicles are microbubbles filled with said physiologically acceptable gas suspended in an aqueous carrier liquid containing phospholipids whose fatty acid residues contain 16 carbons or more.

17. Contrast agents for echography in precursor form consisting of a dry powder comprising lyophilized liposomes and stabilizers, this powder being dispersible in aqueous liquid carriers to form echogenic suspensions of gas-filled microbubbles, characterized in that it is stored under an atmosphere comprising a physiologically acceptable gas whose solubility in water, expressed in liter of gas by liter of water under standard conditions, divided by the square root of the molecular weight does not exceed 0.003.

18. The contrast agent precursors of embodiment 17, in which the liposomes comprise phospholipids whose fatty acid residues have 16 or more carbon atoms.

The following Examples further illustrate various aspects of the invention.

### EXAMPLE 18

Albumin microvesicles filled with air or various gases were prepared as described in EP-A-0324938 using a 10 ml calibrated syringe filled with a 5% human serum albumin (HSA) obtained from the Blood Transfusion Service, Red-Cross Organization, Bern, Switzerland. A sonicator probe (Sonifier Model 250 from Branson Ultrasonic Corp, USA) was lowered into the solution down to the 4 ml mark of the syringe and sonication was effected for 25 sec (energy setting=8). Then the sonicator probe was raised above the solution level up to the 6 ml mark and sonication was resumed under the pulse mode (cycle=0.3) for 40 sec. After standing overnight at 4° C., a top layer containing most of the microvesicles had formed by buoyancy and the bottom layer containing unused albumin debris of denatured protein and other insolubles was discarded. After resuspending the microvesicles in fresh albumin solution the mixture was allowed to settle again at room temperature and the upper layer was finally collected. When the foregoing sequences were carried out under the ambient atmosphere, air filled microballoons were obtained. For obtaining microballoons filled with other gases, the albumin solution was first purged with a new gas, then the foregoing operational sequences were effected under a stream of this gas flowing on the surface of the solution; then at the end of the operations, the suspension was placed in a glass bottle which was extensively purged with the desired gas before sealing.

The various suspensions of microballoons filled with different gases were diluted to 1:10 with distilled water saturated at equilibrium with air, then they were placed in an optical cell as described above and the absorbance was recorded while increasing steadily the pressure over the suspension. During the measurements, the suspensions temperature was kept at 25° C.

The results are shown in the Table 1 below and are expressed in terms of the critical pressure PC values registered for a series of gases defined by names or formulae, the characteristic parameters of such gases, i.e., Mw and water solubility being given, as well as the original bubble count and bubble average size (mean diameter in volume).

TABLE 1

| | | | | Bubble | Bubble | | |
|---|---|---|---|---|---|---|---|
| | | | Solubil- | Count | size | PC | S_{gas}/ |
| Sample | Gas | Mw | ity | (10⁸/ml) | (μm) | (Torr) | Mw^{0.5} |
| AFre1 | CF₄ | 88 | .0038 | 0.8 | 5.1 | 120 | .0004 |
| AFre2 | CBrF₃ | 149 | .0045 | 0.1 | 11.1 | 104 | .0004 |
| ASF1 | SF₆ | 146 | .005 | 13.9 | 6.2 | 150 | .0004 |
| ASF2 | SF₆ | 146 | .005 | 2.0 | 7.9 | 140 | .0004 |
| AN1 | N₂ | 28 | .0144 | 0.4 | 7.8 | 62 | .0027 |
| A14 | Air | 29 | .0167 | 3.1 | 11.9 | 53 | .0031 |
| A18 | Air | 29 | .0167 | 3.8 | 9.2 | 52 | — |
| A19 | Air | 29 | .0167 | 1.9 | 9.5 | 51 | — |
| AMe1 | CH₄ | 16 | .032 | 0.25 | 8.2 | 34 | .008 |
| AKr1 | Kr | 84 | .059 | 0.02 | 9.2 | 86 | .006 |
| AX1 | Xe | 131 | .108 | 0.06 | 17.2 | 65 | .009 |
| AX2 | Xe | 131 | .108 | 0.03 | 16.5 | 89 | .009 |

From the results of Table 1, it is seen that the critical pressure PC increases for gases of lower solubility and higher molecular weight. It can therefore be expected that microvesicles filled with such gases will provide more durable echogenic signals in vivo. It can also be seen that average bubble size generally increases with gas solubility.

### EXAMPLE 19

Aliquots (1 ml) of some of the microballoon suspensions prepared in Example 18 were injected in the Jugular vein of experimental rabbits in order to test echogenicity in vivo. Imaging of the left and right heart ventricles was carried out in the grey scale mode using an Acuson 128-XP5 echography apparatus and a 7.5 MHz transducer. The duration of contrast enhancement in the left ventricle was determined by recording the signal for a period of time. The results are gathered in Table 2 below which also shows the PC of the gases used.

TABLE 2

| | Duration of | |
|---|---|---|
| Sample (Gas) | contrast (sec) | PC (Torr) |
| AMe1 (CH₄) | zero | 34 |
| A14 (air) | 10 | 53 |
| A18 (air) | 11 | 52 |
| AX1 (Xe) | 20 | 65 |
| AX2 (Xe) | 30 | 89 |
| ASF2 (SF₆) | >60 | 140 |

From the above results, one can see the existence of a definite correlation between the critical pressure of the gases tried and the persistence in time of the echogenic signal.

### EXAMPLE 20

A suspension of echogenic air-filled galactose microparticles (Echovist® from Schering AG) was obtained by shaking for 5 sec 3 g of the solid microparticles in 8.5 ml of a 20% galactose solution. In other preparations, the air above a portion of EchovistQ particles was evacuated (0.2 Torr) and replaced by an SF₆ atmosphere, whereby, after addition of the 20% galactose solution, a suspension of microparticles containing associated sulfur hexafluoride was obtained. Aliquots (1 ml) of the suspensions were administered to experimental rabbits (by injection in the jugular vein) and imaging of the heart was effected as described in the previous example. In this case the echogenic microparticles do not transit through the lung capillaries, hence imaging is restricted to the right ventricle and the overall signal persistence has no particular significance. The results of Table 3 below show the value of signal peak intensity a few seconds after injection.

TABLE 3

| | | Signal peak (arbitrary |
|---|---|---|
| Sample No | Gas | units) |
| Gal1 | air | 114 |
| Gal2 | air | 108 |
| Gal3 | SF₆ | 131 |
| Gal4 | SF₆ | 140 |

It can be seen that sulfur hexafluoride, an inert gas with low water solubility, provides echogenic suspensions which generate echogenic signals stronger than comparable suspensions filled with air. These results are particularly interesting in view of the teachings of EP-A-0441468 and EP-A-0357163 (Schering) which disclose the use for echography purposes of microparticles, respectively, cavitate and clathrate compounds filled with various gases including SF₆; these documents do not however report particular advantages of SF₆ over other more common gases with regard to the echogenic response.

### EXAMPLE 21

A series of echogenic suspensions of gas-filled microbubbles were prepared by the general method set forth below:

One gram of a mixture of hydrogenated soya lecithin (from Nattermann Phospholipids GmbH, Germany) and dicetyl-phosphate (DCP), in 9/1 molar ratio, was dissolved in 50 ml of chloroform, and the solution was placed in a 100 ml round flask and evaporated to dryness on a Rotavapor apparatus. Then, 20 ml of distilled water were added and the mixture was slowly agitated at 75° C. for an hour. This resulted in the formation of a suspension of multilamellar liposomes (MLV) which was thereafter extruded at 75° C. through, successively, 3 μm and 0.8 μm polycarbonate membranes (Nuclepore(D)). After cooling, 1 ml aliquots of the extruded suspension were diluted with 9 ml of a concentrated lactose solution (83 g/l), and the diluted suspensions were frozen at −45° C. The frozen samples were thereafter freeze-dried under high vacuum to a free-flowing powder in a vessel which was ultimately filled with air or a gas taken from a selection of gases as indicated in Table 4 below. The powdery samples were then resuspended in 10 ml of water as the carrier liquid, this being effected under a stream of the same gas used to fill the said vessels. Suspension was effected by vigorously shaking for 1 min on a vortex mixer.

The various suspensions were diluted 1:20 with distilled water equilibrated beforehand with air at 25° C. and the dilutions were then pressure tested at 25° C. as disclosed in Example 18 by measuring the optical density in a spectrophotometric cell which was subjected to a progressively increasing hydrostatic pressure until all bubbles had collapsed. The results are collected in Table 4 below which, in addition to the critical pressure PC, gives also the ΔP values (see FIG. 1).

TABLE 4

| | | | | Bubble | | |
|---|---|---|---|---|---|---|
| | | | Solubility | Count | PC | increment |
| Sample No | Gas | Mw | in H₂O | (10⁸/ml) | (Torr) | ΔP (Torr) |
| LFre1 | CF₄ | 88 | .0038 | 1.2 | 97 | 35 |
| LFre2 | CBrF₃ | 149 | .0045 | 0.9 | 116 | 64 |
| LSF1 | SF₆ | 146 | .005 | 1.2 | 92 | 58 |
| LFre3 | C₄F₈ | 200 | .016 | 1.5 | 136 | 145 |
| L1 | air | 29 | .0167 | 15.5 | 68 | 17 |
| L2 | air | 29 | .0167 | 11.2 | 63 | 17 |
| LAr1 | Ar | 40 | .031 | 14.5 | 71 | 18 |
| LKr1 | Kr | 84 | .059 | 12.2 | 86 | 18 |
| LXe1 | Xe | 131 | .108 | 10.1 | 92 | 23 |
| LFre4 | CHClF₂ | 86 | .78 | — | 83 | 25 |

The foregoing results clearly indicate that the highest resistance to pressure increases is provided by the most water-insoluble gases. The behavior of the microbubbles is therefore similar to that of the microballoons in this regard. Also, the less water-soluble gases with the higher molecular weights provide the flattest bubble-collapse/pressure curves (i.e., Δ P is the widest) which is also an important factor of echogenic response durability in vivo, as indicated hereinbefore.

### EXAMPLE 22

Some of the microbubble suspensions of Example 21 were injected to the jugular vein of experimental rabbits as indicated in Example 19 and imaging of the left heart ventricle was effected as indicated previously. The duration of the period for which a useful echogenic signal was detected was recorded and the results are shown in Table 5 below in which C₄F₈ indicates octafluorocyclobutane.

TABLE 5

| | | Contrast duration |
|---|---|---|
| Sample No | Type of gas | (sec) |
| L1 | Air | 38 |
| L2 | Air | 29 |
| LMe1 | CH₄ | 47 |
| LKr1 | Krypton | 37 |
| LFre1 | CF₄ | >120 |
| LFre2 | CBrF₃ | 92 |
| LSF1 | SF₆ | >112 |
| LFre3 | C₄F₈ | >120 |

These results indicate that, again in the case of microbubbles, the gases according to the criteria of the present invention will provide ultrasonic echo signal for a much longer period than most gases used until now.

### EXAMPLE 23

Suspensions of microbubbles were prepared using different gases exactly as described in Example 21, but replacing the lecithin phospholipid ingredient by a mole equivalent of diarachidoylphosphatidylcholine (C₂₀ fatty acid residue) available from Avanti Polar Lipids, Birmingham, Ala. USA. The phospholipid to DCP molar ratio was still 9/1. Then the suspensions were pressure tested as in Example 21; the results, collected in Table 6A below, are to be compared with those of Table 4.

TABLE 6A

| | | | | Bubble | | |
|---|---|---|---|---|---|---|
| Sample | Type of | Mw of | Solubility | Count | PC | increment |
| No | Gas | Gas | in water | (10⁸/ml) | (Torr) | ΔP (Torr) |
| LFre1 | CF₄ | 88 | .0038 | 3.4 | 251 | 124 |
| LFre2 | CBrF₃ | 149 | .0045 | 0.7 | 121 | 74 |
| LSF1 | SF₆ | 146 | .005 | 3.1 | 347 | >150 |
| LFre3 | C₄F₈ | 200 | .016 | 1.7 | >350 | >200 |
| L1 | Air | 29 | .0167 | 3.8 | 60 | 22 |
| LBu1 | Butane | 58 | .027 | 0.4 | 64 | 26 |
| LAr1 | Argon | 40 | .031 | 3.3 | 84 | 47 |
| LMe1 | CH₄ | 16 | .032 | 3.0 | 51 | 19 |
| LEt1 | C₂H₆ | 44 | .034 | 1.4 | 61 | 26 |
| LKr1 | Kr | 84 | .059 | 2.7 | 63 | 18 |
| LXe1 | Xe | 131 | .108 | 1.4 | 60 | 28 |
| LFre4 | CHClF₂ | 86 | .78 | 0.4 | 58 | 28 |

The above results, compared to that of Table 4, show that, at least with low solubility gases, by lengthening the chain of the phospholipid fatty acid residues, one can dramatically increase the stability of the echogenic suspension toward pressure increases. This was further confirmed by repeating the foregoing experiments but replacing the phospholipid component by its higher homolog, i.e., di-phenoylphosphatidylcholine (C₂₂ fatty acid residue). In this case, the resistance to collapse with pressure of the microbubbles suspensions was still further increased.

Some of the microbubbles suspensions of this Example were tested in dogs as described previously for rabbits (imaging of the heart ventricles after injection of 5 ml samples in the anterior cephalic vein). A significant enhancement of the useful in vivo echogenic response was noted, in comparison with the behavior of the preparations disclosed in Example 21, i.e., the increase in chain length of the fatty-acid residue in the phospholipid component increases the useful life of the echogenic agent in vivo.

In the next Table below, there is shown the relative stability in the left ventricle of the rabbit of microbubbles (SF₆) prepared from suspensions of a series of phospholipids whose fatty acid residues have different chain lengths (<injected dose: 1 ml/rabbit).

TABLE 6B

| | Chain | | | |
|---|---|---|---|---|
| | length | PC | increment | Duration of |
| Phospholipid | (Cₙ) | (Torr) | ΔP (Torr) | contrast (sec) |
| DMPC | 14 | 57 | 37 | 31 |
| DPPC | 16 | 100 | 76 | 105 |
| DSPC | 18 | 115 | 95 | 120 |
| DAPC | 20 | 266 | 190 | >300 |

It has been mentioned hereinabove that for the measurement of resistance to pressure described in these Examples, a constant rate of pressure rise of 100 Torr/min was maintained. This is justified by the results given below which show the variations of the PC values for different gases in function to the rate of pressure increase. In these samples DMPC was the phospholipid used.

TABLE 6C

| | PC (Torr) | | |
|---|---|---|---|
| Gas | Rate of pressure increase (Torr/min) | | |
| Sample | 40 | 100 | 200 |
| SF₆ | 51 | 57 | 82 |
| Air | 39 | 50 | 62 |
| CH₄ | 47 | 61 | 69 |
| Xe | 38 | 43 | 51 |
| Freon 22 | 37 | 54 | 67 |

### EXAMPLE 24

A series of albumin microballoons as suspensions in water were prepared under air in a controlled sphere size fashion using the directions given in Example 18. Then the air in some of the samples was replaced by other gases by the gas-exchange sweep method at ambient pressure. Then, after diluting to 1:10 with distilled water as usual, the samples were subjected to pressure testing as in Example 18. From the results gathered in Table 7 below, it can be seen that the two-steps preparation mode gives, in some cases, echo-generating agents with better resistance to pressure than the one-step preparation mode of Example 18.

TABLE 7

| Sample | Type of | Mw of the | Solubility | Initial Bubble | PC |
|---|---|---|---|---|---|
| No | gas | gas | in water | Count (10⁸/ml) | (Torr) |
| A14 | Air | 29 | .0167 | 3.1 | 53 |
| A18 | Air | 29 | .0167 | 3.8 | 52 |
| A18/SF₆ | SF₆ | 146 | .005 | 0.8 | 115 |
| A18/C₂H₆ | C₂H₆ | 30 | .042 | 3.4 | 72 |
| A19 | Air | 29 | .0167 | 1.9 | 51 |
| A19/SF₆ | SF₆ | 146 | .005 | 0.6 | 140 |
| A19/Xe | Xe | 131 | .108 | 1.3 | 67 |
| A22/CF₄ | CF₄ | 88 | .0038 | 1.0 | 167 |
| A22/Kr | Kr | 84 | .059 | 0.6 | 85 |

### EXAMPLE 25

The method of the present invention was applied to an experiment as disclosed in the prior art, for instance Example 1 WO-92/11873. Three grams of Pluronic® F68 (a copolymer of polyoxyethylene-polyoxypropylene with a molecular weight of 8400), 1 g of dipalmitoylphosphatidylglycerol (Na salt, Avanti Polar Lipids) and 3.6 g of glycerol were added to 80 ml of distilled water. After heating at about 80° C., a clear homogenous solution was obtained. The tenside solution was cooled to room temperature and the volume was adjusted to 100 ml. In some experiments (see Table 8) dipalmitoylphosphatidylglycerol was replaced by a mixture of diarachidoylphosphatldylcholine (920 mg) and 80 mg of dipalmitoylphosphatidic acid (Na salt, Avanti Polar lipids).

The bubble suspensions were obtained by using two syringes connected via a three-way valve. One of the syringes was filled with 5 ml of the tenside solution while the other was filled with 0.5 ml of air or gas. The three-way valve was filled with the tenside solution before it was connected to the gas-containing syringe. By alternatively operating the two pistons, the tenside solutions were transferred back and forth between the two syringes (5 times in each direction), milky suspensions were formed. After dilution (1:10 to 1:50) with distilled water saturated at equilibrium with air, the resistance to pressure of the preparations was determined according to Example 18, the pressure increase rate was 240 Torr/min. The following results were obtained:

TABLE 8

| Phospholipid | Gas | Pc (mmHg) | DP (mmHg) |
|---|---|---|---|
| DPPG | air | 28 | 17 |
| DPPG | SF₆ | 138 | 134 |
| DAPC/DPPA 9/1 | air | 46 | 30 |
| DAPC/DPPA 9/1 | SF₆ | 269 | 253 |

It follows that by using the method of the invention and replacing air with other gases, e.g., SF₆, even with known preparations a considerable improvements, i.e., increase in the resistance to pressure, may be achieved. This is true both in the case of negatively charged phospholipids (e.g., DPPG) and in the case of mixtures of neutral and negatively charged phospholipids (DAPC/DPPA).

The above experiment further demonstrates that the recognized problem sensitivity of microbubbles and microballoons to collapse when exposed to pressure, i.e., when suspensions are injected into living beings, has advantageously been solved by the method of the invention. Suspensions with microbubbles or microballoons with greater resistance against collapse and greater stability can advantageously be produced providing suspensions with better reproducibility and improved safety of echographic measurements performed in vivo on a human or animal body.

Further Methods of the Invention and Gas Mixtures Used Therein

Agents used for imaging of the left heart and myocardium should provide clear images and should have good resistance to pressure variation but should not be everlasting and should not disturb images created immediately upon injection. Recirculation is not a desirable feature of agents whose intended use is to cover a range of applications and clear imaging. Obviously, it is highly desirable to modulate the pressure resistance or persistence of the contrast agent after injection, i.e., to use suspensions of bubbles (or microballoons) designed with sufficient pressure resistance but with controlled life-time in the circulation. This demand is fulfilled by the invention using the gas mixtures described below.

Briefly summarized, the invention relates to an injectable ultrasound contrast medium in the form of microbubbles or microballoons comprising at least two biocompatible, at the body temperature gaseous, substances A and B forming a mixture which when in suspension with usual surfactants, additives and stabilizers provides useful ultrasound contrast agents. At least one of the components (B) in the mixture is a gas whose molecular weight is above 80 daltons and whose solubility in water is below 0.0283 ml of gas per ml of water under standard conditions. Gas solubilities referred to below correspond to the Bunsen coefficients and the molecular weights above 80 daltons are considered as relatively high, while the molecular weights below 80 daltons are considered as relatively low. The mixtures of the invention therefore may be defined as mixtures of in which the major portion of the mixture is comprised of “a relatively low” molecular weight gas or gases, while the minor portion of the mixture is comprised of “a relatively high” molecular weight gas or gas mixture. The quantity of this “minor” or activating component (B) in the contrast medium is practically always between 0.5 and 41 volume percent. The other component (A) of the ultrasound contrast media may be a gas or a mixture of gases whose solubility in water is above that of nitrogen (0.0144 ml/ml of water under standard conditions) and whose quantity in the mixture is practically always in a proportion of between 59-99.5% by vol. This “major” or dominating component is preferably a gas or gases whose molecular weights are relatively low, usually below 80 daltons, and is chosen from gases such as oxygen, air, nitrogen, carbon dioxide or mixtures thereof.

In the ultrasound contrast medium of the invention the gas whose molecular weight is above 80 daltons may be a mixture of gases or mixture of substances which are gaseous at body temperature but which, at ambient temperatures, may be in the liquid state. Such gaseous or liquid substances may be useful in the contrast media of the invention as long as the molecular weight of each such substance is greater than 80 daltons and the solubility in water of each substance is below 0.0283 ml of gas per ml of water under standard conditions.

When filled with the contrast media of the invention and dispersed in an aqueous carrier containing usual surfactants, additives and stabilizers, the microbubbles formed provide an injectable contrast agent for ultrasonic imaging, of controlled resistance to pressure variations and modulated persistence after injection. In addition to the microbubbles, the contrast agent of the invention will contain surfactants stabilizing the microbubble evanescent gas/liquid envelope, and optionally, hydrophilic agents and other additives. The additives may include block copolymers of polyoxypropylene and polyoxyethylene (poloxamers), polyoxyethylenesorbitans, sorbitol, glycerol-polyalkylene stearate, glycerolpolyoxyethylene ricinoleate, homo- and copolymers of polyalkylene glycols, soybean-oil as well as hydrogenated derivatives, ethers and esters of sucrose or other carbohydrates with fatty acids, fatty alcohols, glycerides of soya-oil, dextran, sucrose and carbohydrates. Surfactants may be film forming and non-film forming and may include polymerizable amphiphilic compounds of the type of linoleyl-lecithins or polyethylene dodecanoate. Preferably, the surfactants comprise one or more film forming surfactants in lamellar or laminar form selected between phosphatidic acid, phosphatidylcholine, phosphatidylethanolamine, phosphatidylserine, phosphatidylglycerol, phosphatidylinositol, cardiolipin, sphingomyelin and mixtures thereof.

The invention also comprises a method of making the ultrasound contrast agents by suspending in a physiologically acceptable carrier containing usual surfactants and stabilizers, gas filled microbubbles or microballoons comprising a mixture of gases at least one of which is a gas whose minimum effective amount in the mixture may be determined according to the expression:
B_{c} %=K/e^{b Mwt}+C
in which B_{c} % (by vol.) is the total quantity of the component B in the mixture, K, C & b are constants with values of 140, −10.8 and 0.012 respectively, M_{wt} represents the molecular weight of the component B exceeding 80. The contrast agents made according to the present method comprise suspensions of microbubbles or microballoons with excellent resistance to pressure variations and a controlled resorption rate.

The invention also includes a kit comprising a dry formulation which is usually stored under a mixture of gases and/or liquids that are converted into gases at body temperature. When dispersed in a physiologically acceptable carrier liquid, the dry formulation with the mixture of gases and/or liquids produces the ultrasound contrast agent of the invention. A method of storage of the dry lyophilised formulation in the presence of the ultrasound contrast media is also disclosed.

The invention further comprises a method of making contrast agents with microbubbles containing the ultrasound contrast media, as well as their use in imaging of organs in human or animal body.

### BRIEF DESCRIPTION OF THE DRAWINGS

FIG. 1 is a graph relating bubble concentration (bubble count), expressed in terms of optical density in the aforementioned range, and the pressure applied over the bubble suspension.

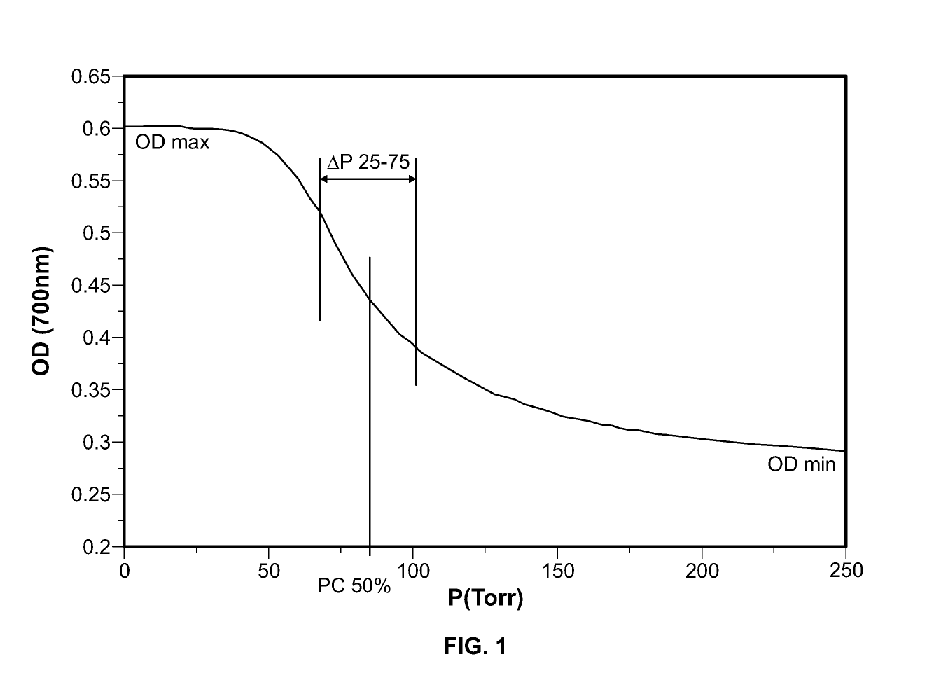

FIG. 2 is a schematic presentation of an ultrasound contrast medium according to the invention.

FIG. 3 is schematic diagram of the critical pressure (Pc) of the contrast medium as a function of the quantity of a chosen gas in the mixture.

FIG. 4 represents a diagram of the critical pressure (Pc) of a contrast medium made with octafluorocyclobutane (C₄F₈) and dodecafluoropentane (C₅F₁₂) as a function of quantity of gas in the mixture.

FIG. 5 is a diagram of the minimum amount of a gas in the mixture as a function of the molecular weight.

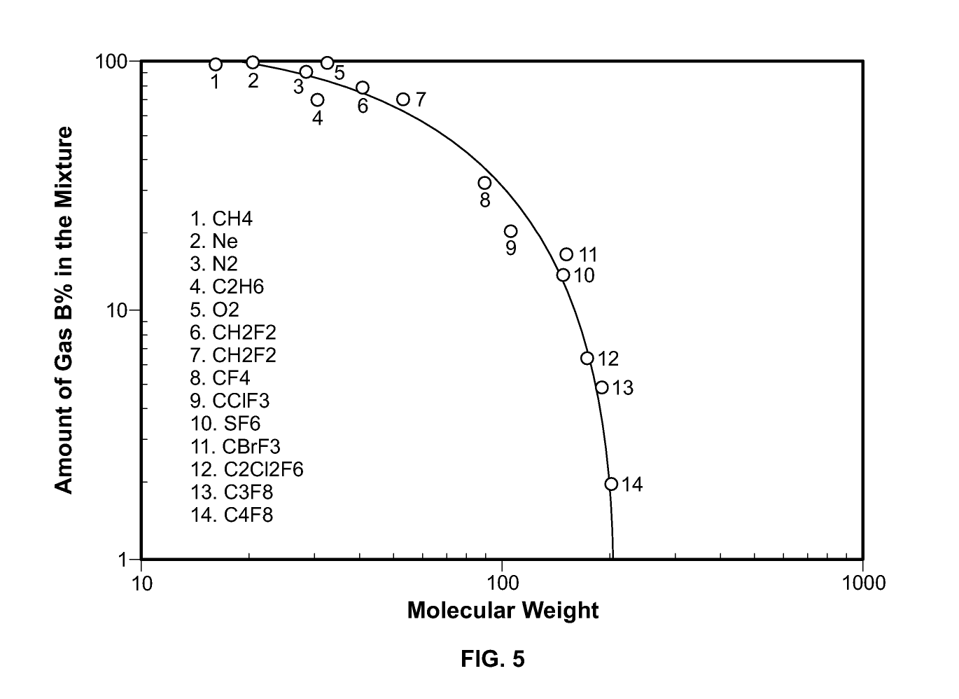

FIG. 6 is a graphic representation of the in vivo echographic responses obtained as a function of time in the left ventricle of a minipig after intravenous injection of contrast media containing various concentrations of SF₆.

FIG. 7 represents a diagram of in vivo echographic response obtained as a function of time with contrast media containing various concentrations of C₄F₈.

FIG. 8 is graphical presentation of echographic responses as a function of the microbubble concentration for a freshly prepared suspension

This invention is based on the unexpected finding that an ultrasound contrast medium comprising bubbles filled with a mixture of at least two biocompatible gaseous or at body temperature gaseous substances A (major or a relatively low molecular weight) and B (activating or a relatively high molecular weight), will provide, in suspension with usual surfactants, additives and stabilizers, injectable ultrasound contrast agents that combine desirable resistance to pressure and a shorter life time in the circulation, both of these parameters being controllable at will. As long as at least one of the (activating) substances or components in the mixture with molecular weight greater than 80 daltons (relatively high molecular weight) is present in certain minimal proportion and as long as its solubility in water is below 0.0283 ml of gas per ml of water at standard conditions, the ultrasound contrast medium will provide echographic properties as good as that obtained when using the pure substances alone. By “activating” it is meant the substance or component which imparts its physical properties to the other components in the mixture rendering the mixture, in terms of echogenicity and resistance to pressure variations, behave the same or almost the same as the substance or component alone (in pure form). The quantity of the first, activating or high molecular weight, component in the contrast medium in most cases vary from as low as 0.5 volume percent (for substances with high molecular weight and low solubility in water) to 41 volume percent. The experiments have shown that substances with the molecular weight below 80 daltons (“low molecular weight”) are not suitable as the activating components and that the upper limit of the molecular weight is difficult to establish as all compounds tested were effective as long as their molecular weight was relatively high, i.e., above 80. Thus compounds with the molecular weight of about 240 daltons such as decafluorobutane or 290 daltons such as perfluoropentane have been found as effective activating component. Also there are indications that substances such as 1,2,3-nonadecane tricarboxylic acid, 2-hydroxy-trimethylester with the molecular weight sightly over 500 daltons may also be used as an activating, high molecular weight, component. The other “major” component is correspondingly present in an amount of 59 to 99.5% by volume and may be a gas or gases whose solubility in water is greater than that of nitrogen (0.0144 ml/ml of water under standard conditions). The second component is preferably oxygen, air, nitrogen, carbon dioxide or mixtures thereof and more preferably oxygen or air. However, for the component A, other less common gases like argon, xenon, krypton, CHClF₂ or nitrous oxide may also be used. Some of these less common gases may have molecular weights higher than that of O₂, N₂, air, CO₂, etc., for instance above 80 daltons but, in this case, their solubility in water will exceed that of the gases of category B. i.e., will be above 0.0283 ml/ml of water.

It was quite unexpected to find that suspending in an aqueous carrier a mixture formed of as little as 0.5% by volume of a substance such as dodecafluoropentane, or 0.8% by volume of decafluorobutane in admixture with air will produce microbubbles giving excellent echographic images in vivo and resistance to pressure variations. This is particularly surprising since it was heretofore considered necessary that in order to obtain good echographic images of the left heart and the myocardium, these substances, and for that matter a number of others, be used at 100% concentrations, i.e., in pure form (without air). Experiments with mixtures containing different amounts of these, low water solubility, substances and air have shown that the echographic images are as good as those obtained under similar conditions using echographic agents made with only pure substances.

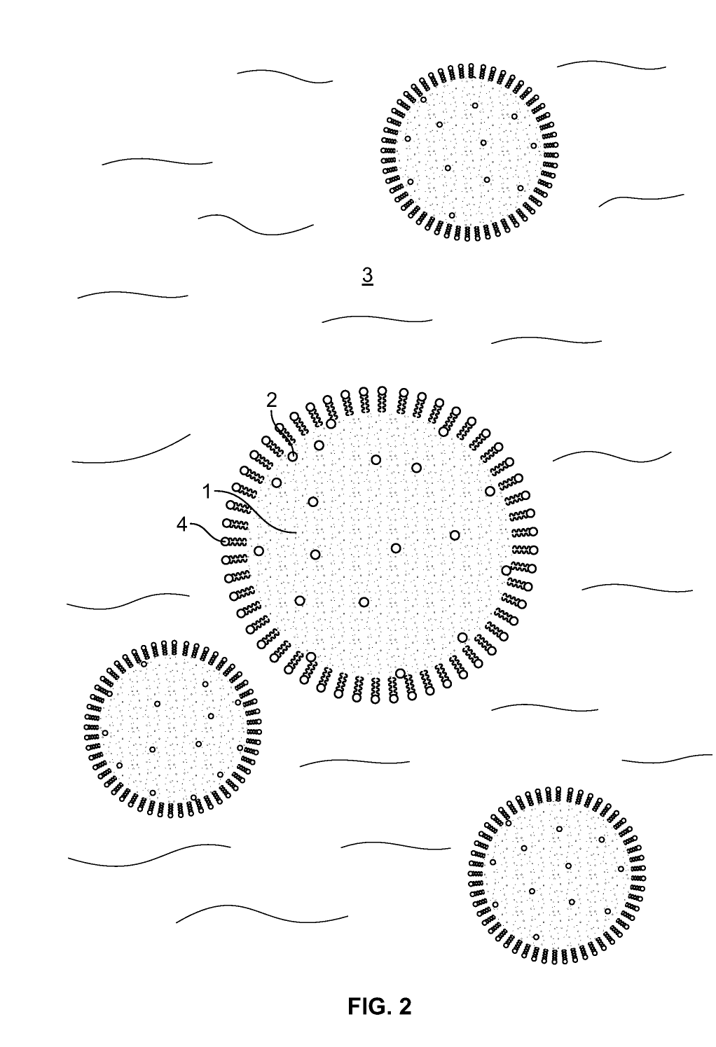

Early studies have shown that rapid elimination of air microbubbles in the circulation takes place because this otherwise physiologically preferred gas is quickly resorbed by dilution and that evanescence of the microbubbles may be reduced through the use of various surfactants, additives and stabilizers. In the early days of development, as a cure to the evanescence problem, microballoons or microvesicles with a material wall have also been proposed. Microvesicles with walls made from natural or synthetic polymers such as lipid bilayers (liposomes) or denatured proteins like albumin filled with air or CO₂ have been proposed. The poor resistance to pressure variations and the consequent loss of echogenicity of the older contrast agents has inspired a search for gaseous particles with greater resistance to the pressure variations occurring in the blood stream. Hence, filler gases such as sulfur hexafluoride of more recently dodecafluoropentane have been proposed. Experimentation with these gases have indicated that upon injection, the suspensions of microbubbles made with these gases taken alone are indeed very resistant to collapse in the blood circulation. As a result of these initial findings, close to 200 different gases have been identified as potentially useful for making ultrasound contrast agents. It has thus been unexpectedly found that by mixing oxygen or air with some of these gases resistant to pressure one may obtain ultrasound agents which will have physiologically better tolerance and/or shorter resorption half-life than pure sulfur hexafluoride or dodecafluoropentane, still retaining the good pressure resistance of these gases when taken alone. It is postulated that such surprising behavior of the ultrasound medium of the invention comes from the fact that in the microbubbles containing the gas mixtures diffusion of air into surrounding liquid is slowed by the presence of the large molecules of gas or gases whose solubilities in water are about the same or lower than that of air or oxygen. Although the reasons for this surprising behavior are yet unexplained, it can be postulated that the molecules of the high molecular weight gas, even though in very minor amount, do actually “plug the holes” in the microbubbles boundary and thus prevent escape of the low molecular weight gas by transmembrane diffusion. A graphical presentation of this model is shown in the FIG. 2 where the microbubble containing air (1) admixed with a gas whose molecular weight is above 80 daltons (2) is suspended in an aqueous medium (3). The evanescent outer layer (4) stabilized by a surfactant (e.g., phospholipid) keeps the gas mixture within contained volume defining the microbubble. The activating or minority gas B being uniformly dispersed through out the microbubble volume will have a slower diffusion and ultimately will block the pores of, in the aqueous solution spontaneously formed surfactant membrane-like envelope, thus preventing rapid departure of the smaller and typically more soluble majority component A. On the other hand, the activating or minor component gas (B) exhibit greater affinity for the lipophilic part of the surfactant used for stabilization of the evanescent envelope than oxygen or air. Thus according to another hypothesis these gases tend to concentrate in the vicinity of the membrane preventing or slowing diffusion of the smaller gas(es) across the membrane. Be that as it may, the experimental data gathered suggest that for preparation of echographic media of the invention, the required amount of the activating gas in the mixture is that which corresponds to blocking the porosity of the given membrane material or to the amount required for a monomolecular layer formed on the inner wall of the microbubbles. Therefore, the minimum amount required is that which is needed to block the pores or cover the inner wall of the membrane to prevent escape and resorption of the low molecular weight component.

It is also believed that the superior properties of the ultrasound contrast medium of the invention comes from the combined use of nitrogen, carbon dioxide, oxygen or air (essentially an oxygen/nitrogen mixture) with other gases. Functionally, these biologically and physiologically compatible gases provide important characteristics of the media in question thus ensuring their advantageous properties. Although, the ultrasound contrast media of the invention may be made with a number of other gases serving as the majority or component A, oxygen and air are preferred. In the context of this document air is treated as a “one component” gas.

According to the invention, ultrasound contrast media with high resistance to pressure variations combined with relatively rapid resorption, i.e., clearance in the body can be obtained when using a gas or gases whose molecular weights is/are above 80 daltons in admixture with gas or gases whose solubilities in water are greater than 0.0144 ml/ml of water and molecular weight(s) is/are usually below 80 daltons. Gases such as oxygen or air mixed with substances which are gases at the body temperature but which at the ambient temperatures may be in the liquid state will produce echographic media that will possess all advantages of the gases in the mixture. In other words these mixtures when injected as suspensions of microbubbles will provide clear and crisp images with sharp contrasts (typical for microbubbles with good resistance to pressure variations) and at the same time will be resorbed substantially as easily as if filled with air or oxygen only. Thus by combining air, nitrogen, carbon dioxide or oxygen with a certain controlled amount of any of the known biocompatible high molecular weight substances which at the body temperature are gases, ultrasound contrast media with important and totally unexpected advantages are obtained. As explained above, these media provide the best of each components, i.e., a good resistance to pressure variations from one and a relatively rapid resorption from the other and at the same time eliminating respective disadvantages of each component taken alone in the media. This is particularly surprising as one would have expected properties averaging those of the components taken separately.

As long as the molecular weight of such biocompatible substances (B) is greater than 80 daltons and their solubility in water is below 0.0283 ml of gas per ml of water under standard conditions, such substances in the gaseous or liquid state are useful for the contrast media of the invention. Although in conjunction with suitable surfactants and stabilizers, gases like sulfur hexafluoride, tetrafluoromethane, chlorotrifluoromethane, dichlorodifluoro-methane, bromotrifluoromethane, bromochlorodifluoromethane, dibromo-difluoromethane dichlorotetrafluoroethane, chloropentafluoroethane, hexafluoroethane, hexafluoropropylene, octafluoropropane, hexafluoro-butadiene, octafluoro-2-butene, octafluorocyclobutane, decafluorobutane, perfluorocyclopentane, dodecafluoropentane and more preferably sulfur hexafluoride and/or octafluorocyclobutane, may be used in category B, the media of the invention preferably contains as gas B a gas selected from sulfur hexafluoride, tetrafluoromethane, hexafluoroethane, hexafluoro-propylene, octafluoropropane, hexafluorobutadiene, octafluoro-2-butene, octafluorocyclobutane, decafluorobutane, perfluorocyclopentane, dodecafluoropentane and more preferably sulfur hexafluoride and/or octafluorocyclobutane.

Another unexpected and surprising feature of the invention is the fact that when the criteria of WO 93/05819 are applied to the media of the present invention the Q coefficient obtained with the present gas mixtures is below 5. This is astounding since, according to WO 93/05819 media with Q coefficients below 5 are to be excluded from gases suitable for preparing useful ultrasound contrast media. Nevertheless, it has been found that the uniform gas mixtures of the present invention although having a Q coefficient well below 5, still provide contrast agents useful for ultrasound imaging.

When filled with the contrast media of the invention and dispersed in an aqueous carrier containing usual surfactants, additives and stabilizers, the microbubbles formed provide a useful contrast agent for ultrasonic imaging. In addition to the microbubbles, the contrast agent of the invention will contain surfactants additives and stabilizers. Surfactants which may include one or more film forming surfactants in lamellar or laminar form are used to stabilize the microbubble evanescent gas/liquid envelope. Hydrating agents and/or hydrophilic stabilizer compounds such as polyethylene glycol, carbohydrates such as lactose or sucrose, dextran, starch, and other polysaccharides or other conventional additives like polyoxypropylene glycol and polyoxyethylene glycol; ethers of fatty alcohols with polyoxyalkylene glycols; esters of fatty acids with polyoxyalkylated sorbitan; soaps; glycerol-polyalkylene stearate; glycerol-polyoxyethylene ricinoleate; homo- and copolymers of polyalkylene glycols; polyethoxylated soya-oil and castor oil as well as hydrogenated derivatives; ethers and esters of sucrose or other carbohydrates with fatty acids, fatty alcohols, these being optionally polyoxyakylated; mono-, di- and triglycerides of saturated or unsaturated fatty acids; glycerides of soya-oil and sucrose may also be used. Surfactants may be film forming and non-film forming and may include polymerizable amphiphilic compounds of the type of linoleyl-lecithins or polyethylene dodecanoate. Preferably, the surfactants are film forming and more preferably are phospholipids selected from phosphatidic acid, phosphatidylcholine, phosphatidylethanolamine, phosphatidylserine, phosphatidylglycerol, phosphatidylinositol, cardiolipin, sphingomyelin and mixtures thereof.

It is understood that the invention is not limited to the contrast agents in which only microbubbles are used as carriers of the ultrasound contrast media of the invention. Any suitable particle filled with the ultrasound contrast medium, e.g., liposomes or microballoons having an envelope produced from synthetic or natural polymers or proteins may conveniently be used. Thus it has been established that microballoons prepared with albumin, or liposome vesicles or iodipamide ethyl ester porous particles when filled with the ultrasound contrast media of the invention, provide good echographic contrast agents. Suspensions in which the microbubbles were stabilized with sorbitol or non-ionic surfactants such as polyoxyethylene/polyoxypropylene copolymers (commercially known as Pluronic®) have demonstrated equally good imaging capability when compared to that of the original formulations made with the pure substances taken alone. It is therefore, believed that the invention offers a more generalized concept of ultrasound media and offers better insight into the problems of ultrasound imaging as well as better control of contrast agent properties. The media and contrast agents containing the media of the invention are, therefore, considered as products which take the technique one step further in its development.

The invention also comprises a method of making the ultrasound contrast agent, in which a gas mixture of at least two components is suspended in a physiologically acceptable aqueous carrier liquid containing usual surfactants and stabilizers so as to form gas filled microbubbles or microballoons, characterized in that the minimum effective proportion of at least one gas component (B) in said mixture of gases is determined according to the criteria
B_{c} %=K/e^{b Mwt}+C
in which B_{c} % (by vol.) is the total quantity of the component B in the mixture, K & C are constants with values of 140 and −10.8 respectively, M_{wt} represents the molecular weight of the component B exceeding 80 and b is quantity that is a complex function of operating temperature and thickness of the membrane (a lipid film) that stabilizes the microbubbles; however, since the body temperature is substantially constant and the stabilizer film structure substantially independent of lipid concentration, the value of b keeps in the interval 0.011-0.012 and it may be considered as constant. The contrast agents made according to the method comprise suspensions of microbubbles or microballoons with excellent resistance to pressure variations and a relatively rapid resorption. Both of the properties are controlled to the extent that practically custom-tailored echographic agents are now possible. With the above criteria it is possible to produce an agent with desired characteristics starting from any available non-toxic (“off the shelf”) substance which at body temperature is gas and which has the molecular weight and solubility in water as explained above.

The invention also includes a dry formulation comprising surfactants, additives and stabilizers stored under a mixture of substances which at the body temperature are gases at least one of which is a gas whose molecular weight is greater than 80 daltons and whose solubility in water is below 0.0283 ml per ml of water under standard conditions. Prior to injection the formulation comprising lyophilised film forming surfactants and optionally, hydrating agents like polyethylene glycol or other conventional hydrophilic substances, is admixed with a physiologically acceptable carrier liquid to produce the ultrasound contrast agent of the invention. The film forming surfactant is, preferably, a phospholipid selected from phosphatidic acid, phosphatidylcholine, phosphatidylethanolamine, phosphatidylserine, phosphatidylglycerol, phosphatidylinositol, cardiolipin, sphingomyelin and mixtures thereof.

In a variant, stabilization of the microbubble evanescent gas/liquid envelope may be secured by non-ionic surfactants such as copolymers of polyoxyethylene and polyoxypropylene in combination with a film forming surfactant such as dipalmitoylphosphatidylglycerol. As before the aqueous liquid carrier may further contain hydrophilic additives such as glycerol, PEG, sorbitol, etc. Furthermore, useful agents of the invention may be prepared with saline solutions containing Tween® 20 (Polyethylene Oxide Sorbitan ester), sorbitol, soybean oil, and optionally other additives.

Also disclosed is a two-component kit comprising as the first component a dry formulation of surfactants, additives and stabilizers stored under a mixture of gases and as the second component a physiologically acceptable carrier liquid which when brought in contact with the first component provides an ultrasound contrast media. The kit may include a system of two separate vials, each containing one of the components, which are interconnected so that the components may be conveniently brought together prior to use of the contrast agent. Clearly, the vial containing the dry formulation will at the same time contain the ultrasound medium of the invention. Conveniently, the kit may be in the form of a pre-filled two compartment syringe and may further include means for connecting a needle on one of its ends.

The invention further comprises a method of making contrast agents with microbubbles containing the ultrasound contrast media, as well as their use in imaging of organs in human or animal body.

When used for imaging of organs in human or animal body the ultrasound contrast medium of the invention is administered to the patient in the form of an aqueous suspension in the above described physiologically acceptable carrier liquid and the patient is scanned with an ultrasound probe whereby an image of the organ or the part of the body imaged is produced.

The invention described herein can be further elucidated by the description of the following representative (but not limiting) embodiments numbered 1-21:

1. An ultrasound contrast medium comprising substances gaseous at body temperature which when in suspension in an aqueous carrier liquid containing usual surfactants, additives and stabilizers provide agents for ultrasound echography, characterized in that the medium is a mixture of gases (A) and (B) at least one of which is a gas (B) whose molecular weight is greater than 80 daltons and whose solubility in water is below 0.283 ml of gas per ml of water at standard conditions.

2. The ultrasound contrast medium of embodiment 1, wherein proportion of gas B in the mixture is 0.5-41% by vol. and the proportion of gas A is 59-99.5% by vol.

3. The ultrasound contrast medium of embodiment 1 or 2, wherein the gas with molecular weight above 80 daltons is selected from the group consisting of SF₆, CF₄, C₂F₆, C₂F₈, C₃F₆, C₃F₈, C₄F₆, C₄F₈, C₄F₁₀, C₅F₁₀, C₅F₁₂ and mixtures thereof.

4. The ultrasound contrast medium of embodiment 3, wherein the gas B is sulfur hexafluoride or octafluorocyclobutane.

5. The ultrasound contrast medium of embodiment 1 or 2, wherein the gas A is selected from the group consisting of air, oxygen, nitrogen, carbon dioxide and mixtures thereof.

6. An ultrasound contrast agent consisting of a suspension of gas filled microbubbles or microballoons in a physiologically acceptable aqueous carrier comprising usual surfactants, additives and stabilizers, characterized in that the gas is a mixture of at least two gases A and B in which at least one gas (B) has a molecular weight greater than 80 daltons and solubility in water is below 0.0283 ml per ml of water at standard conditions.

The ultrasound contrast agent of embodiment 6, wherein the mixture contains 0.5-41% by vol. of gas B and 59-99.5% by vol. of gas A.

8. The ultrasound contrast agent of embodiment 6 or 7, wherein the gas B with molecular weight above 80 daltons is selected from the group consisting of SF₆, CF₄, C₂F₆, C₂F₈, C₃F₆, C₃F₈, C₄F₆, C₄F₈, C₄F₁₀, C₅F₁₀, C₅C₁₂ and mixtures thereof.

9. The ultrasound contrast agent of embodiment 7, wherein the gas A is selected from the group consisting of air, oxygen, nitrogen, carbon dioxide or mixtures thereof.

10. The ultrasound contrast agent of embodiment 7, wherein the surfactants comprise at least one film forming surfactant present in laminar and/or lamellar form and, optionally, hydrophilic stabilizers.

11. The ultrasound contrast agent of embodiment 7, wherein the film forming surfactant is a phospholipid.

12. The ultrasound contrast agent of embodiment 7, wherein the phospholipid is selected from the group consisting of phosphatidic acid, phosphatidylcholine, phosphatidylethanolamine, phosphatidylserine, phosphatidylglycerol, phosphatidylinositol, cardiolipin, sphingomyelin, and mixtures therein.

13. The ultrasound contrast agent of embodiment 11, wherein in addition to the phospholipid the aqueous carrier comprises copolymers of polyoxyethylene and polyoxypropylene, and glycerol.

14. The ultrasound contrast agent of embodiment 7, wherein the surfactants, additives and stabilizers comprise soy bean oil and Tween® and sorbitol.

15. A dry formulation comprising surfactants, additives and stabilizers stored under a mixture of substances which at body temperature are gases at least one of which is a gas whose molecular weight is greater than 80 daltons and whose solubility in water is below 0.0283 ml per ml of water at standard conditions.

16. A two component kit comprising as the first component a dry formulation of surfactants, additives and stabilizers stored under a mixture of gases and as the second component a physiologically acceptable carrier liquid which when admixed with the first component provides, as a suspension of the two components, an ultrasound contrast medium, characterized in that at least one of the gases in the mixture is a gas whose molecular weight is greater than 80 daltons and whose solubility in water is below 0.028 ml of gas per ml of water at standard conditions.

17. A method of making the ultrasound contrast agent of embodiment 7, in which a gas mixture of at least two components (A and B) is suspended in a physiologically acceptable aqueous carrier liquid containing usual surfactants, additives and stabilizers so as to form, gas filled microbubbles or microballoons, characterized in that the minimum effective proportion of at least one gas component in said mixture of gases is determined according to the criteria
B_{c} %=K/eb^{bMwt}+C
in which B_{c} % (by vol.) is the total quantity of the component B in the mixture, K, C and b are constants with values of 140, −10.8 and 0.012 respectively, M_{wt} represents the molecular weight of the component B which is >80.

18. The method of making the ultrasound contrast agent of embodiment 17, wherein the surfactant is a phospholipid selected from the group consisting of phosphatidic acid, phosphatidyl-choline, phosphatidylethanolamine, phosphatidylserine, phosphatidyl-glycerol, phosphatidylinositol, cardiolipin, sphingomyelin and mixtures thereof.

19. Use of the ultrasound contrast medium of embodiment 1 for the manufacture of contrast agents useful in imaging the cardiovascular systems of humans or animals.

20. Use of the ultrasound contrast medium of embodiment 1 for the manufacture of ultrasound contrast agents.

21. Use of the ultrasound contrast agent of embodiment 1 for imaging of human or animal body.

The following examples further illustrate the invention:

### EXAMPLE 26

Multilamellar vesicles (MLVs) were prepared by dissolving 120 mg of diarachidoylphosphatidylcholine (DAPC, from Avanti Polar Lipids) and 5 mg of dipalmitoylphosphatidic acid (DPPA acid form, from Avanti Polar Lipids) in 25 ml of hexane/ethanol (8/2, v/v) then evaporating the solvents to dryness in a round-bottomed flask using a rotary evaporator. The residual lipid film was dried in a vacuum desiccator and after addition of water (5 ml), the mixture was incubated at 90° C. for 30 minutes under agitation. The resulting solution was extruded at 85° C. through a 0.8 μm polycarbonate filter (Nuclepore®). This preparation was added to 45 ml of a 167 mg/ml solution of dextran 10,000 MW (Fluka) in water. The solution was thoroughly mixed, transferred in a 500 ml round-bottom flask, frozen at −45° C. and lyophilised under 13.33 Nt/m² (0.1 Torr). Complete sublimation of the ice was obtained overnight. Aliquots (100 mg) of the resulting lyophilisate were introduced in 20 ml glass vials. The vials were closed with rubber stoppers and the air removed from vials using vacuum. Mixtures of air with various amounts of sulfur hexafluoride were introduced into the vials via a needle through the stopper.

Bubble suspensions were obtained by injecting in each vial 10 ml of a 3% glycerol solution in water followed by vigorous mixing. The resulting microbubble suspensions were counted using a hemacytometer. The mean bubble size was 2.0 μm. In vitro measurements (as defined in EP-A-0 554 213) of the critical pressure (Pc), echogenicity (i.e., backscatter coefficient) and the bubble count for various samples were performed (see Table 9).

TABLE 9

| Sam- | air | SF₆ | Q | PC | Echogenicity | Concentration |
|---|---|---|---|---|---|---|
| ple | % vol | % vol | coeff. | mmHg | 1/(cm · sr) × 100 | (bubbles/ml) |
| A | 100 | 0 | 1.0 | 43 | 1.6 | 1.5 × 10⁸ |
| B | 95 | 5 | 1.3 | 68 | 2.1 | 1.4 × 10⁸ |
| C | 90 | 10 | 1.6 | 85 | 2.4 | 1.5 × 10⁸ |
| D | 75 | 25 | 3.1 | 101 | 2.3 | 1.4 × 10⁸ |
| E | 65 | 35 | 4.7 | 106 | 2.4 | 1.5 × 10⁸ |
| F | 59 | 41 | 5.8 | 108 | 2.4 | 1.6 × 10⁸ |
| G | 0 | 100 | 722.3 | 115 | 2.3 | 1.5 × 10⁸ |

As it may be seen from the results, the microbubbles containing 100% air (sample A) have a low resistance to pressure. However, with only 5% SF₆, the resistance to pressure increases considerably (sample B). With 25% SF₆ the resistance to pressure is almost identical to that of 100% SF₆. On the other hand, the bubble concentrations, the mean bubble sizes and the backscatter coefficients are almost independent of the percentage of SF₆.

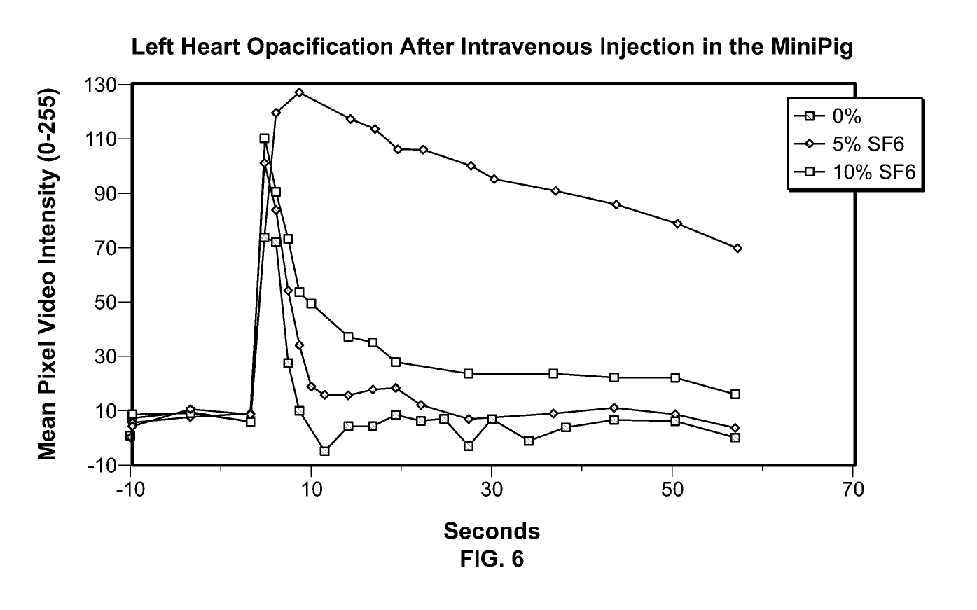

The resulting suspensions were injected intravenously into minipigs (Pitman Moore) at a dose of 0.5 ml per 10 kg and the images of the left ventricular cavity were recorded on a video recorder. In vivo echographic measurements were performed using an Acuson XP128 ultrasound system (Acuson Corp. USA) and a 7 MHz sector transducer. The intensity of the contrast was measured by video densitometry using an image analyzer (Dextra Inc.). FIG. 6 shows the video densitometric recordings in the left heart of a minipig. Again a considerable difference is observed between the 100% air case (sample A) and the 95% air case (sample B). In particular, with 5% SF₆ the maximum intensity is already almost achieved and the half life in circulation shows also a very rapid increase. With 10% SF₆, there is no additional increase in intensity but only a prolongation of the half-life. From the example, it follows that using more than 10% to 25% SF₆ in the gas mixture provides no real benefit. It is interesting to note that the values of the Q coefficient obtained for the mixtures used were well below the critical value of 5 stipulated by WO-A-93/05819.

### EXAMPLE 27

Aliquots (25 mg) of the PEG/DAPC/DPPA lyophilisate obtained as described in Example 26 (using PEG 4000 instead of dextran 10,000) were introduced in 10 ml glass vials. Tedlar® sampling bags were filled with air and octafluorocyclobutane (C₄F₈). Known volumes were withdrawn from the bags by syringes and the contents thereof were mixed via a three way stopcock system. Selected gas mixtures were then introduced into the glass vials (previously evacuated). The lyophilisates were then suspended in 2.5 ml saline (0.9% NaCl). The results presented below show the resistance to pressure, the bubble concentration and the backscatter coefficient of the suspensions. In the case of 100% C₄F₈ the resistance to pressure reached to 225 mm Hg (compared to 43 mm Hg in the case of air). Again a considerable increase in pressure resistance was already observed with only 5% C₄F₈ (Pc=117 mmHg).

TABLE 10

| Sam- | air | C₄F₈ | Q | PC | Echogenicity 1/ | Concentration |
|---|---|---|---|---|---|---|
| ple | % vol | % vol | coeff. | mmHg | (cm · sr) × 100 | (bubbles/ml) |
| A | 100 | 0 | 1.0 | 43 | 1.6 | 1.8 × 10⁸ |
| B | 95 | 5 | 1.4 | 117 | 2.2 | 3.1 × 10⁸ |
| C | 90 | 10 | 1.7 | 152 | 3.1 | 4.7 × 10⁸ |
| D | 75 | 25 | 3.3 | 197 | 3.5 | 4.9 × 10⁸ |
| E | 65 | 35 | 4.6 | 209 | 3.4 | 4.3 × 10⁸ |
| F | 59 | 41 | 5.5 | 218 | 2.8 | 4.0 × 10⁸ |
| G | 0 | 100 | 1531 | 225 | 2.3 | 3.8 × 10⁸ |

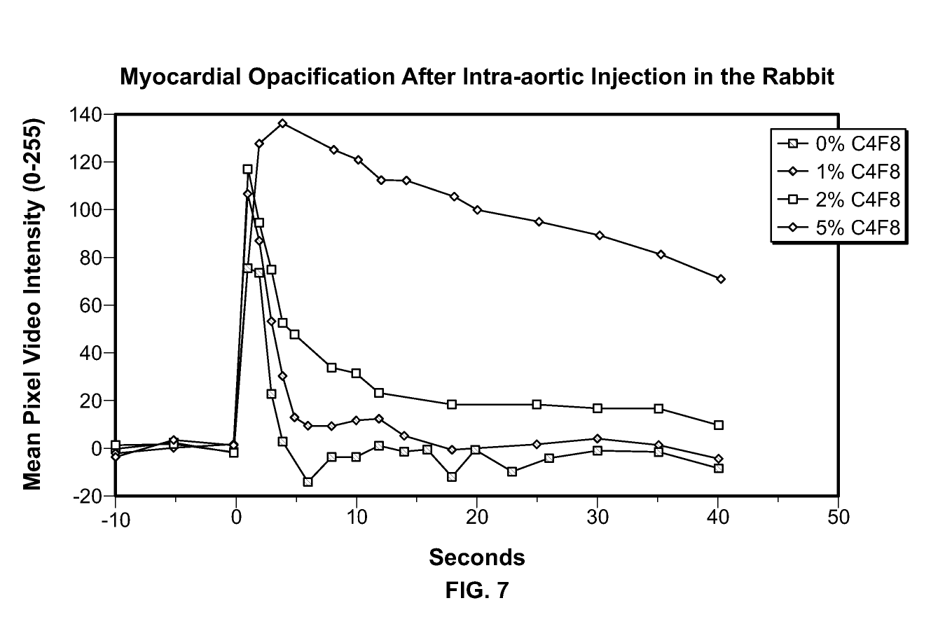

After intra-aortic injection in rabbits (0.03 ml/kg), a slight prolongation of the contrast effect in the myocardium was noticed already with 2% C₄F₈ (when compared to air). However with 5% C₄F₈, the duration of the contrast increased considerably as if above a threshold value in the resistance to pressure, the persistence of the bubbles increases tremendously (see FIG. 7).

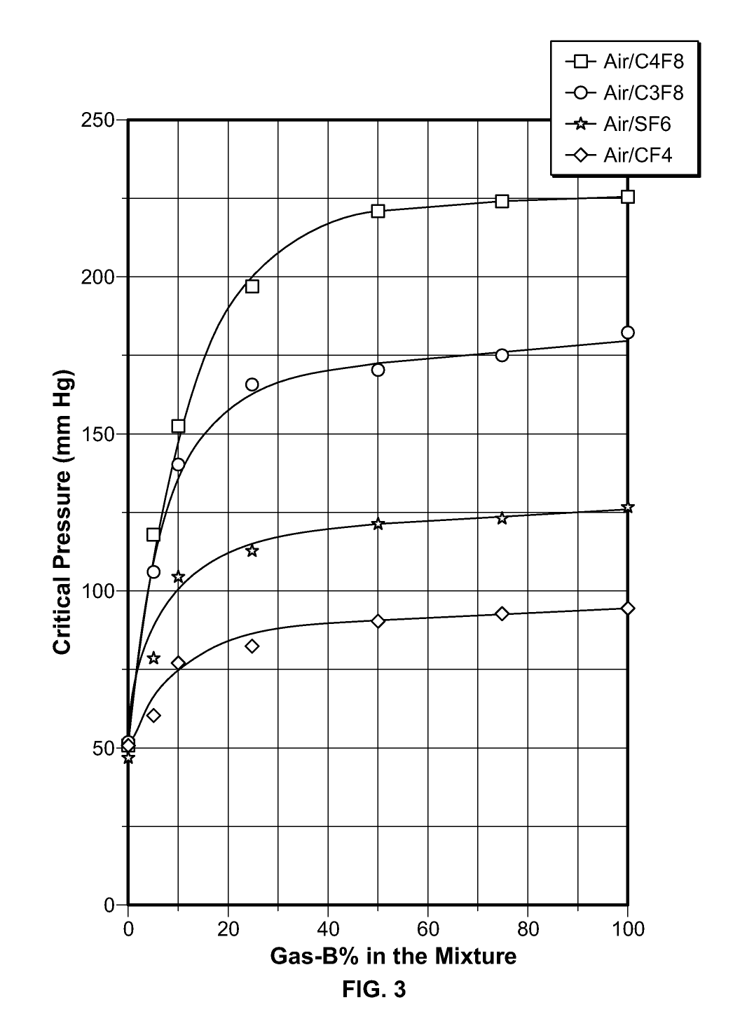

N Here again, this combination of gases provided very good images at 5% of gas B in the mixture, while excellent images of the left heart were obtained with the mixtures containing up to 25% of octafluorocyclobutane. Corresponding diagram of critical pressure as a function of C₄F₈ in the mixture with air is given in FIG. 3. This example again shows that the use of mixture of gases allows to improve considerably the resistance to pressure of air bubbles simply by adding a small percentage of a high molecular weight/low solubility gas. The figure further shows that by appropriate selection of the gas mixture it becomes possible to obtain any desired resistance to pressure.

### EXAMPLE 28

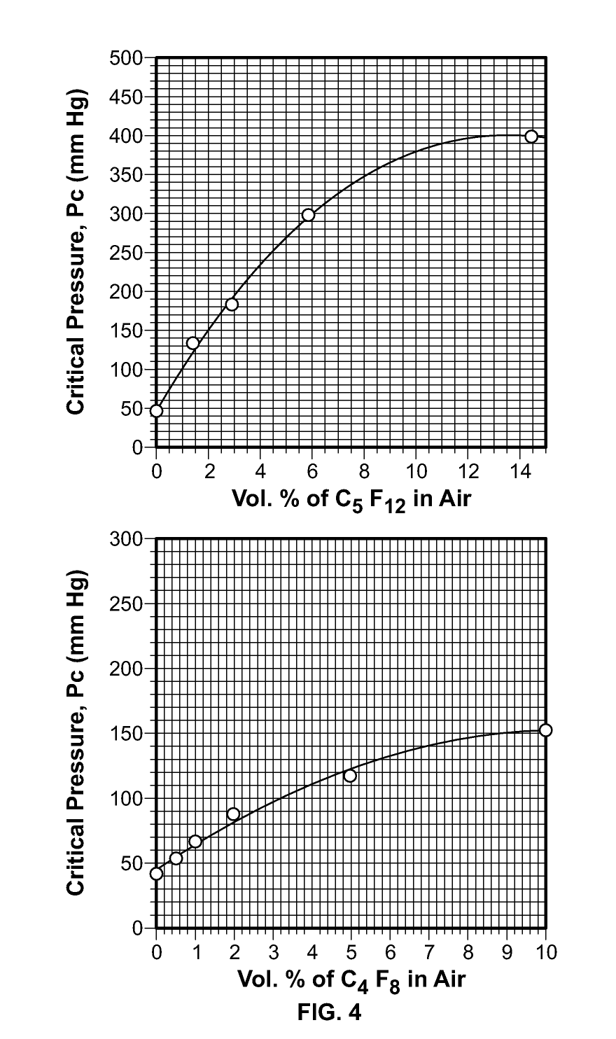

The same lyophilisate as that described in Example 30 was used. The gas phase was made of dodecafluoropentane (C₅F₁₂) and air. C₅F₁₂ is a liquid at room temperature with a boiling point of 29.5° C. 24 ml glass vials each containing 50 mg of the PEG/DSPC/DPPG lyophilisate obtained as described in Example 30 were put under vacuum, closed under vacuum, then heated at 45° C. Small volumes (a few microliters) of C₅F₁₂ were injected in the vials still at 45° C. through the stopper. Air was then introduced to restore atmospheric pressure in the vials. After cooling at room temperature, saline (5 ml) was injected through the stopper and the vials were vigorously agitated. The actual percentage of C₅F₁₂ in the gas phase was calculated assuming full vaporization of the liquid introduced. This is an overestimate as at this temperature part of the liquid will not be in gaseous state. As shown in FIG. 4 an increase in the resistance to pressure could already be detected with only 0.5% C₅F₁₂ in air. At 1.4% C₅F₁₂ the resistance to pressure exceeded 130 mm Hg. These suspensions were also injected intravenously into minipigs (0.5 ml per 15 kg). Intensity was measured by videodensitometry as described in Example 26. As shown in Table 11, maximum intensity was already obtained with 1.4% C₅F₁₂.

TABLE 11

| | | | | | | | | Inten | |
|---|---|---|---|---|---|---|---|---|---|
| | air % | C₅F₁₂ % | Q | Pc | Echogen | Conc. | half-life | Gray | AUC |
| Sample | vol | vol | coeff. | mmHg | (cm · sr)⁻¹ | (bub/ml) | (t_{1/2}) sec | level | (t_{1/2}) |
| A | 100 | 0 | 1.0 | 43 | 0.017 | 1.8 × 10⁸ | 11 | 22 | 78 |
| B | 99.5 | 0.5 | 1.0 | 80 | — | — | — | — | — |
| C | 98.6 | 1.4 | 1.1 | 133 | 0.026 | 3.9 × 10⁸ | 14 | 97 | 609 |
| D | 97.1 | 2.9 | 1.4 | 182 | 0.028 | 3.9 × 10⁸ | 17 | 98 | 860 |
| E | 94.2 | 5.8 | 1.7 | 295 | 0.040 | 5.2 × 10⁸ | 59 | 99 | 3682 |
| F | 85.5 | 4.5 | 3.4 | 394 | 0.036 | 4.5 × 10⁸ | 78 | 97 | 5141 |

*Estimated

Higher percentages of C₅F₁₂ result into prolongation of the half life and increase in the AUC. The half life (t_{1/2}) was determined as the time elapsed between injection and the time at which the intensity had dropped to 50% of its maximum value. The area under the curve (AUC) was measured until t_{1/2}. The Examples 26-28 also demonstrate that contrary to the statements made in WO-A-93/05819 it is possible to obtain outstanding contrast enhancing agents from gas mixtures whose Q values are smaller and in certain cases much smaller than 5.

### EXAMPLE 29

Fifty eight milligrams of diarachidoylphosphatidylcholine (DAPC), 2.4 mg of dipalmitoylphosphatidic acid (DPPA) both from Avanti Polar Lipids (USA) and 3.94 g of polyethyleneglycol (PEG 4000 from Siegfried) were dissolved at 60° C. in tert-butanol (20 ml) in a round-bottom glass vessel. The clear solution was rapidly cooled at −45° C. and lyophilized. Aliquots (25 mg) of the white cake obtained were introduced in 10 ml glass vials.

Tedlar® gas sampling bags were filled with gases, one with air and one with sulfur hexafluoride (SF₆). Pre-determined volumes of the gases were collected from each bag through the septum by using two separate syringes and the contents mixed via a three way stopcock. The resulting gas mixtures were introduced into 10 ml glass vials which were evacuated and closed with rubber stopper while still under vacuum. Seven vials contained gas mixtures of air and SF₆ in different proportions. The concentration of SF₆ was between 0 to 100%. The actual percentage of SF₆ in the gas phase was confirmed by densitometry (A. Paar densimeter). Saline (0.9% NaCl) was then injected through the stopper into each vial (5 ml per vial) and the powder dissolved by vigorous shaking. The resulting microbubble suspensions were evaluated in vitro and in vivo. The resistance to pressure P_{c} was determined using a nephelometric assay and the backscatter coefficient was measured using a pulse echo set up (both described in EP-A-0 554 213). The bubble concentration and mean bubble size were determined by analysis with a Coulter Multisizer II (Coulter Electronics Ltd). The results obtained were virtually the same to those given for Example 26.

### EXAMPLE 30

A PEG/DSPC/DPPG lyophilisate was prepared as described in Example 29 using 30 mg of distearoylphosphatidylcholine (DSPC) and 30 mg dipalmitoyl-phosphatidylglycerol (DPPG) (both from SYGENA, Switzerland). Aliquots (25 mg) of the resulting cake were introduced in 10 ml glass vials. Different gas mixtures were introduced in various vials by withdrawing appropriate volumes from Tedlar® bags filled with the various gases. Table 12 shows the gas mixtures investigated, their molecular weight and their solubilities (expressed as Bunsen coefficient) and the resistance to pressure of the microbubbles obtained.

TABLE 12

| | | | | Gas | Gas | Solu- | Solu- |
|---|---|---|---|---|---|---|---|
| Gas | Gas | Gas B | Pc | A | B | bility* | bility* |
| A | B | % vol | mmHg | M_{wt} | M_{wt} | Gas A | Gas B |
| O₂ | C₄F₈ | 0 | 40 | 32 | 200 | 0.083 | 0.016 |
| | C₄F₈ | 5 | 112 | | | | |
| | C₄F₈ | 10 | 148 | | | | |
| CO₂ | C₄F₈ | 0 | 50 | 44 | 200 | 0.74 | 0.016 |
| | C₄F₈ | 5 | — | | | | |
| | C₄F₈ | 10 | 204 | | | | |
| CHClF₂ | C₄F₈ | 0 | — | 86.5 | 200 | 0.78 | 0.016 |
| | C₄F₈ | 5 | 106 | | | | |
| | C₄F₈ | 10 | 163 | | | | |
| Xenon | C₄F₈ | 0 | 50 | 131 | 200 | 0.108 | 0.016 |
| | C₄F₈ | 5 | 147 | | | | |
| | C₄F₈ | 10 | 181 | | | | |
| SF₆ | C₄F₈ | 0 | 124 | 146 | 200 | 0.005 | 0.016 |
| | C₄F₈ | 5 | 159 | | | | |
| | C₄F₈ | 10 | 193 | | | | |
| N₂ | SF₆ | 0 | 55 | 28 | 146 | 0.0144 | 0.005 |
| | SF₆ | 5 | 80 | | | | |
| | SF₆ | 10 | 108 | | | | |
| CF₄ | SF₆ | 0 | 84 | 182 | 146 | 0.0038 | 0.005 |
| | SF₆ | 5 | 91 | | | | |
| | SF₆ | 10 | 106 | | | | |
| Xenon | SF₆ | 0 | 50 | 131 | 146 | 0.108 | 0.005 |
| | SF₆ | 5 | 67 | | | | |
| | SF₆ | 10 | 83 | | | | |

*Bunsen coefficient

It is particularly interesting to note that highly soluble gases such as CO₂, xenon, CHClF₂ which alone are very poor in their ability to form stable and resistant bubbles are nevertheless able to give rise to highly stable bubbles provided a small percentage of a gas such as SF₆ or C₄F₈ is added.

### EXAMPLE 31

The method of the invention was applied to a microbubble suspension prepared as described in Example 1 of WO 92/11873. Three grams of Pluronic® F68 (a copolymer of polyoxyethylene-polyoxypropylene with a molecular weight of 8400), 1 g of dipalmitoylphosphatidylglycerol and 3.6 g of glycerol were added to 80 ml of distilled water. After heating at about 80° C. a clear homogenous solution was obtained. The tenside solution was cooled to room temperature and the volume adjusted to 100 ml. The bubble suspension was obtained by using two syringes connected via a three-way valve. One of the syringes was filled with 5 ml of the tenside solution while the other was filled with 0.5 ml of air or air/C₄F₈ mixture (see Table 13). The three way valve was filled with the tenside solution before it was connected to the gas-containing syringe. By alternatively operating the two pistons, the tenside solution was transferred back and forth between the two syringes (5 times in each direction) and milky suspensions were obtained. After dilution (1/50) in distilled water saturated with air the resistance to pressure (Pc) was determined. Aliquots were injected intravenously into anaesthetized rabbits (0.03 ml/kg) and echographic images of the left ventricle were recorded. The area under the curve (AUC) as well as the half life (t_{1/2}) were determined. A considerable increase of the half-life and AUC was observed when using 5% C₄F₈ (compared to air). Similar results were obtained with 5% C₅F₁₂.

TABLE 13

| | | | right ventr. | | | left ventr. | | |
|---|---|---|---|---|---|---|---|---|
| air | C₄F₈ | Pc | opacif. | | | opacif. | | |
| % vol | % vol | (mm-Hg) | t_{1/2} | intens | AUC | t_{1/2} | intens | AUC |
| 100 | 0 | 54 | 4 | 96 | 280 | 9 | 101 | 514 |
| 99 | 1 | 89 | 7 | 98 | 377 | 12 | 98 | 632 |
| 95 | 5 | 136 | 14 | 94 | 829 | 40 | 101 | 2693 |
| air | C₅F₁₂ | | | | | | | |
| 95 | 5 | 177 | * | * | * | 43 | 111 | 3249 |

* Shadowing

### EXAMPLE 32

A suspension of microbubbles was obtained as described in WO-A-93/05819 using mixtures of air and octafluorocyclobutane C₄F₈. An aqueous solution containing sorbitol (20 g), NaCl (0.9 g), soybean oil (6 ml), Tween 20 (0.5 ml) was prepared and adjusted to 100 ml of distilled water. 10 ml of this solution was taken up in a 10 ml syringe. A second 10 ml syringe was filled with mixtures of air and C₄F₈. The two syringes were connected via a three way stopcock. By operating alternatively each of the two pistons for a total of 20 times, milky suspensions were obtained. These suspensions were tested for their resistance to pressure. Aliquots were also injected intravenously into anaesthetized rabbits (0.1 ml/kg) and echographic images of the left ventricle were recorded. Interestingly no contrast was detected in the left ventricle with 1% or even 5% C₄F₈. However, left ventricle opacification was obtained with 1% and even more with 5% of C₅F₁₂.

TABLE 14

| | | | left | | | right | left |
|---|---|---|---|---|---|---|---|
| air | C₄F₈ | Right ventr. | ventr. | air | C₅F₁₂ | ventr. | ventr. |
| % vol | % vol | opacif. | opacif. | % vol | % vol | opacif. | opacif. |
| 100 | 0 | + | − | 100 | 0 | + | − |
| 99 | 1 | + | − | 99 | 1 | + | + |
| 95 | 5 | ++ | − | 95 | 5 | ++ | ++ |

“−” no opacification
“+” moderate opacification
“++” good opacification

### EXAMPLE 33

A PEG/DSPC/DPPG lyophilisate was prepared as described in Example 29 using 30 mg of distearoylphosphatidylcholine (DSPC) and 30 mg dipalmitoyl-phosphatidylglycerol (DPPG) (both from SYGENA, Switzerland). Aliquots (25 mg) of the resulting cake were introduced in 10 ml glass vials. Different gas mixtures were introduced in various vials by withdrawing appropriate volumes from Tedlar® bags filled with the various gases. Table 15 shows the gas mixtures investigated and the resistance to pressure of the microbubbles obtained.

TABLE 15

| | C₄F₈ | CF₄ | Air | Pc | |
|---|---|---|---|---|---|
| Sample | % vol | % vol | % vol | mmHg | Absorbance |
| A1 | 5 | 15 | 80 | 113 | 0.284 |
| A2 | 10 | 10 | 80 | 147 | 0.281 |
| A3 | 15 | 5 | 80 | 167 | 0.281 |

It is noteworthy the high molecular weight gas may even be a mixture of two or more gases with high molecular weight and solubility (expressed as Bunsen coefficient) which is below 0.0283. It follows that in place of a single gas (B), mixtures of two or more activating or minor component gases may also be used. Although, in this example, the critical pressure is proportional to the percentage of the heavier of the two components, it is believed that other combinations of gases may further lower the total amount of the insoluble gas(es) in the mixture through synergy.

Further Stable Microbubbles Suspensions

A further aspect of the present invention is based on the unexpected finding that very stable suspensions of gas filled microbubbles comprising at least 10⁷ microbubbles per millilitre may be obtained using phospholipids as stabilizers even if very low concentrations thereof are employed. The suspensions usable as contrasting agents in ultrasonic echography are obtained by suspending in an aqueous carrier at least one phospholipid as a stabiliser of the microbubbles against collapse with time and pressure, the concentration of the phospholipids being below 0.01% wt. but equal to or higher than that at which the phospholipid molecules are present solely at the gas microbubble-liquid interface.

It was quite unexpected to discover that as negligible amounts of the phospholipid surfactants involved here (used alone or with a relatively small proportions of other amphiphiles) can so effectively stabilize microbubbles. In the presence of other amphipathic compounds (such as Pluronic®) the mutual cohesion between stabilizer molecules is apparently decreased and formation of monomolecular phospholipid films is inhibited. However, in the absence of large amounts of other amphiphilic agents, the unhindered intermolecular binding forces (electrostatic interaction or hydrogen bonding) between phospholipid molecules are sufficient to ensure formation of stable film-like structures stabilizing the bubbles against collapse or coalescence.

According to the invention, suspensions of high microbubble concentration, high stability, long storage capacity and ease of preparation may be obtained even if the concentrations of surfactants and other additives in the suspensions are kept well below the levels used in the state-of-the-art formulations. The amount of phospholipids used in the compositions of the invention may be as low as about that only necessary for formation of a single monolayer of the surfactant around the gas microbubbles while the concentration of the bubbles in the suspension is maintained above 10⁷ microbubbles per millilitre. According to the present aspect of the invention, microbubbles with a liposome-like double layer of surfactant (gas filled liposomes) are not likely to exist and have not been observed. Instead, as discussed in more detail infra, the microbubbles are bounded by a mono-molecular layer of surfactant molecules.

The invention further includes dry formulations which may be used to generate the injectable suspensions of the invention by simply mixing with an aqueous carrier phase. These dry formulations are stable when stored over time and at temperatures above ambient temperature. Indeed, the preferred dry formulations of the invention may be reconstituted to generate injectable suspensions of gas filled microbubbles whose echogenicity is unaffected even after storage for a month at 40° C.

Suspensions with high microbubble concentrations e.g. between 10⁹ and 10¹⁰ bubbles/ml of relatively high stability and long storage capacity may be prepared even if the concentration of the phospholipid surfactants are kept well below the levels known in the art. Suspensions with as little as 1 μg of phospholipids per ml may be prepared as long as the amount of the surfactants used is not below that which is necessary for formation of a single monolayer of the lipids around the gas microbubbles and as long as they are produced according to one of the methods herein disclosed.

Calculations have shown that for bubble concentrations of 10⁸ bubbles/ml depending on the size distribution of the microbubbles this concentration may be as low as 1 μg/ml or 0.0001%, however, the phospholipid concentrations between 0.0002% and up to 0.01% are preferred. More preferably the concentration of the phospholipids in the stable suspensions of microbubbles of the invention is between 0.001% and 0.009%. Although further reduction of the amount of phospholipids in the suspension is possible, suspensions prepared with less than 0.0001% wt. are unstable, their total bubble count is low and their echographic response upon injection is not satisfactory. On the other hand, suspensions prepared with more than 0.01% of phospholipids upon injection do not perform better i.e. their stability and echographic response do not further improve with the concentration. Thus, the higher concentrations may only increase the probability of undesirable side effects as set out in the discussion of the prior art. It is tentatively postulated that only the segments of the surfactants which are in the lamellar or laminar form can effectively release molecules organized properly to stabilize the bubbles. This may explain why the concentration of the surfactant may be so low without impairing the stability of the gas bubbles.

The suspensions of the invention offer important advantages over the compositions of the prior art not only because of the low phospholipid content but also because the total amount of injected solutes i.e. lipids and/or synthetic polymers and other additives is between 1,000 and 50,000 times lower than heretofore. This is achieved without any loss of microbubble concentration i.e. echogenicity or stability of the product. In addition to the very low concentration of solutes, the invention provides suspensions which may contain only the microbubbles whose contribution to the echographic signal is relatively significant i.e. suspensions which are free of any microbubbles which do not actively participate in the imaging process.

Needless to say that with such low concentrations of solutes in the injectable composition of the invention probability of undesirable side effects is greatly reduced and elimination of the injected agent is significantly improved.

The microbubble suspensions with low phospholipid content of the invention may be prepared from the film forming phospholipids whose structure has been modified in a convenient manner e.g. by freeze-drying or spray-drying solutions of the crude phospholipids in a suitable solvent. Prior to formation of the suspension by dispersion in an aqueous carrier the freeze dried or spray dried phospholipid powders are contacted with air or preferably, another gas discussed herein, such as a fluorinated gas. When contacted with the aqueous carrier the powdered phospholipids whose structure has been disrupted will form lamellarized or laminarized segments which will stabilise the microbubbles of the gas dispersed therein. Conveniently, the suspensions with low phospholipid content of the invention may also be prepared with phospholipids which were lamellarized or laminarized prior to their contacting with air or another gas. Hence, contacting the phospholipids with air or another gas may be carried out when the phospholipids are in a dry powder form or in the form of a dispersion of laminarized phospholipids in the aqueous carrier.

The term lamellar or laminar form indicates that the surfactants are in the form of thin films or sheets involving one or more molecular layers. As described in WO-A-91/15244 conversion of film forming surfactants into lamellar form can easily be done by, for example, any liposome forming method, for instance by high pressure homogenisation or by sonication under acoustical or ultrasonic frequencies. The conversion into lamellar form may also be performed by coating microparticles (10 μm or less) of a hydrosoluble carrier solid (NaCl, sucrose, lactose or other carbohydrates) with a phospholipid with subsequent dissolution of the coated carrier in an aqueous phase. Similarly, insoluble particles, e.g. glass or resin microbeads may be coated by moistening in a solution of a phospholipid in an organic solvent following by evaporation of the solvent. The lipid coated microbeads are thereafter contacted with an aqueous carrier phase, whereby liposomic vesicles will form in the carrier phase. Also, phospholipids can be lamellarized by heating slightly above critical temperature (Tc) and gentle stirring. The critical temperature is the temperature of gel-to-liquid transition of the phospholipids.

Practically, to produce the low phospholipid content suspensions of microbubbles according to the invention, one may start with liposome suspensions or solutions prepared by any known technique as long as the liposomic vesicles are “unloaded”, i.e. they do not have encapsulated therein any foreign material but the aqueous phase of the solution itself.

The introduction of gas into a liposome solution can be effected by usual means, injection i.e. forcing gas through tiny orifices into the liposome solution, or simply dissolving the gas in the solution by applying pressure and then suddenly releasing the pressure. Another way is to agitate or sonicate the liposome solution in the presence of physiologically acceptable gas. Also one can generate the formation of a gas within the solution of liposomes itself, for instance by a gas releasing chemical reaction, e.g. decomposing a dissolved carbonate or bicarbonate by acid.

When laminarized surfactants are suspended in an aqueous liquid carrier and gas is introduced to provide microbubbles, the microbubbles become progressively surrounded and stabilised by a monomolecular layer of surfactant molecules and not a bilayer as in the case of liposome vesicles. This structural rearrangement of the surfactant molecules can be activated mechanically (agitation) or thermally. The required energy is lower in the presence of cohesion releasing agents, such as Pluronic®. On the other hand, presence of the cohesion releasing agents in the microbubble formulations reduces the natural affinity between phospholipid molecules having as a direct consequence a reduced stability of the microbubbles to external pressures (e.g. above 20-30 Torr).

As already mentioned, to prepare the low phospholipid content suspensions of the invention, in place of phospholipid solutions, one may start with dry phospholipids which may or may not be lamellarized. When lamellarized, such phospholipids can be obtained for instance by dehydrating liposomes, i.e. liposomes which have been prepared normally by means of conventional techniques in the form of aqueous solutions and thereafter dehydrated by usual means. One of the methods for dehydrating liposomes is freeze-drying (lyophilization), i.e. the liposome solution, preferably containing hydrophilic compounds, is frozen and dried by evaporation (sublimation) under reduced pressure.

In another approach, non-lamellarized or non-laminarized phospholipids may be obtained by dissolving the phospholipid in an organic solvent and drying the solution without going through liposome formation. In other words, this can be done by dissolving the phospholipids in a suitable organic solvent together with a hydrophilic stabiliser substance e.g. a polymer like PVP, PVA, PEG (preferably the PEG polymer has a molecular weight from about 1000 to about 7500, with a molecular weight from about 2000 to about 5000 being preferred and PEG 4000 being most preferred), etc. or a compound soluble both in the organic solvent and water and freeze-drying or spray-drying the solution. Further examples of the hydrophilic stabiliser compounds soluble in water and the organic solvent are malic acid, glycolic acid, maltol and the like. Any suitable organic solvent may be used as long as its boiling point is sufficiently low and its melting point is sufficiently high to facilitate subsequent drying. Typical organic solvents would be for instance dioxane, cyclohexanol, tertiary butanol, tetrachlorodifluoro ethylene (C₂Cl₄F₂) or 2-methyl-2-butanol however, tertiary butanol, 2-methyl-2-butanol and C₂Cl₄F₂ are preferred. In this variant the criteria used for selection of the hydrophilic stabiliser is its solubility in the organic solvent of choice. The suspensions of microbubbles are produced from such powders using the same steps as with powders of the laminarized phospholipids.

Similarly, prior to effecting the freeze-drying of pre-lamellarized or pre-laminarized phospholipid solutions, a hydrophilic stabiliser compound is dissolved in the solution. However, here the choice of the hydrophilic stabilisers is much greater since a carbohydrate like lactose or sucrose as well as a hydrophilic polymer like dextran, starch, PVP, PVA, PEG and the like may be used.

Hydrophilic stabilizer compounds also aid in homogenising the microbubbles size distribution and enhance stability under storage. Actually making very dilute aqueous solutions (0.0001-0.01% by weight) of freeze-dried phospholipids stabilised with, for instance, a 10:1 to 1000:1 weight ratio of polyethyleneglycol to lipid enables to produce aqueous microbubbles suspensions counting 10⁹-10¹⁰ bubbles/ml (size distribution mainly 0.5-10 μm) which are stable, without significant observable change, even when stored for prolonged periods. This is obtained by simple dissolution of the dried laminarized phospholipids which have been stored under gas without shaking or any violent agitation. The freeze-drying technique under reduced pressure is very useful because it permits, restoration of the pressure above the dried powders with any of the physiologically acceptable gases discussed infra, i.e. nitrogen, CO₂, argon, methane, freons (organic compounds containing one or more carbon atoms and fluorine), SF₆, CF₄, etc., whereby after redispersion of the phospholipids processed under such conditions suspensions of microbubbles containing the above gases are obtained. It has been found that the surfactants which are convenient in this invention can be selected from amphipathic compounds capable of forming stable films in the presence of water and gases. The preferred surfactants include the lecithins (phosphatidylcholine) and other phospholipids, inter alia phosphatidic acid (PA), phosphatidylinositol phosphatidylethanolamine (PE), phosphatidyl-serine (PS), phosphatidylglycerol (PG), cardiolipin (CL), sphingomyelins. Examples of suitable phospholipids are natural or synthetic lecithins, such as egg or soya bean lecithin, or saturated synthetic lecithins, such as, dimyristoylphosphatidylcholine, dipalmitoylphosphatidylcholine, distearoylphosphatidylcholine or diarachidoylphosphatidylcholine or unsaturated synthetic lecithins, such as dioleylphosphatidyl choline or dilinoleylphosphatidylcholine, with saturated lecithins being preferred.

Additives like cholesterol and other substances can be added to one or more of the foregoing lipids in proportions ranging from zero to 50% by weight. Such additives may include other non-phospholipid surfactants that can be used in admixture with the film forming surfactants and most of which are known. For instance, compounds like polyoxypropylene glycol and polyoxyethylene glycol as well as various copolymers thereof. Other additives may include the acid form of phospholipids (such as phosphatidylglycerol and phosphatidic acid), dicetylphosphate, fatty acids, ergosterol, phytosterol, sitosterol, lanosterol, tocopherol, propyl gallate, ascorbyl palmitate and butylated hydroxytoluene. The amount of these non-film forming surfactants are usually up to 50% by weight of the total amount of surfactants but preferably between 0 and 30%. Again this means that the concentration of the various additives in the low phospholipid content suspensions of the invention are in the range of 0-0.05% which is more than one hundred times less than in the compositions known so far.

It should also be mentioned that another feature of the suspensions of the invention is a relatively “high” gas entrapping capacity of the microbubbles i.e. high ratio between the amount of the surfactant and the total amount of the entrapped gas. Hence, with suspensions in which the microbubbles have sizes in the 1 to 5 μm range, it is tentatively estimated that the weight ratio of phospholipids present at the gas bubble-liquid interface to the volume of entrapped gas under standard conditions is between 0.1 mg/ml and 100 mg/ml.

In practice all injectable compositions should also be as far as possible isotonic with blood. Hence, before injection, small amounts of isotonic agents may also be added to the suspensions of the invention. The isotonic agents are physiological solutions commonly used in medicine and they comprise aqueous saline solution (0.9% NaCl), 2.6% glycerol solution, 5% dextrose solution, etc.

The invention further concerns a method of making stable suspensions of microbubbles usable as contrast agents in ultrasonic echography. Basically, the method comprises adapting the concentration of the phospholipids in the suspension of microbubbles stabilized by said phospholipids to a selected value. Usually, one will start with a microbubble suspension containing more phospholipids than the value desired and one will reduce the amount of said phospholipids relatively to the volume of gas entrapped in the microbubble, without substantially reducing the count of echo generating bubbles. This can be done, for instance, by removing portions of the carrier liquid containing phospholipids not directly involved at the gas/liquid interface and diluting the suspension with fresher carrier liquid. For doing this, one may create within the suspension region (a) where the echo generating bubbles will gather and region (b) where said bubbles are strongly diluted. Then the liquid in region (b) can be withdrawn by separation by usual means (decantation, siphoning, etc.) and a comparable volume of fresh carrier liquid is supplied for replenishment to the suspension. This operation can be repeated one or more times, whereby the content in phospholipids not directly involved in stabilizing the bubbles will be progressively reduced.

It is generally not desirable to achieve complete removal of the phospholipid molecules not present at the bubble gas/liquid interface as some unbalance from equilibrium may result, i.e. if the depletion is advanced too far, some surfactant molecules at the gas/liquid interface may be set free with consequent bubble destabilization. Experiments have shown that the concentration of phospholipids—in the carrier liquid may be substantially decreased down without significant changes in properties and adverse effects. This means that, actually, the optimal phospholipid concentration (within the given limits) will be rather dictated by the type of application i.e. if relatively high phospholipid concentrations are admissible, the ideal concentration value will be near the upper limit of the range. On the other hand, if depending on the condition of the patient to be diagnosed, the absolute value of phospholipids must be further reduced, this can be done without adverse effects regarding microbubble count and echogenic efficiency.

An embodiment of the method comprises selecting a film forming surfactant and optionally converting it into lamellar form using one of the methods known in the art or disclosed hereinbefore. The surfactant is then contacted with gas and admixed with an aqueous liquid carrier in a closed container whereby a suspension of microbubbles will form. The suspension is allowed to stand for a while and a layer of gas filled microbubbles formed is left to rise to the top of the container. The lower part of the mother liquor is then removed and the supernatant layer of microbubbles washed with an aqueous solution saturated with the gas used in preparation of the microbubbles. This washing can be repeated several times until substantially all unused or free surfactant molecules are removed. Unused or free molecules means all surfactant molecules that do not participate in formation of the stabilising monomolecular layer around the gas microbubbles.

In addition to providing the low phospholipid content suspensions, the washing technique offers an additional advantage in that it allows further purification of the suspensions of the invention, i.e. by removal of all or almost all microbubbles whose contribution to the echographic response of the injected suspension is relatively insignificant. The purification thus provides suspensions comprising only positively selected microbubbles, i.e. the microbubbles which upon injection will participate equally in the reflection of echographic signals. This leads to suspensions containing not only a very low concentration of phospholipids and other additives, but free from any microbubbles which do not actively participate in the imaging process.

In a variant of the method, the surfactant which optionally may be in lamellar form, is admixed with the aqueous liquid carrier prior to contacting with gas.

The invention described herein can be further elucidated by the description of the following representative (but not limiting) embodiments numbered 1-22:

1. An injectable suspension of gas filled microbubbles in an aqueous carrier liquid, usable as contrast agent in ultrasonic echography, comprising at least 10⁷ microbubbles per millilitre and amphipathic compounds at least one of which is a phospholipid stabilizer of the microbubbles against collapse, characterized in that the concentration of the phospholipids in the carrier liquid is below 0.01% by weight while being equal to or above that at which the phospholipid molecules are present solely at the gas microbubble-liquid interface.

2. The injectable suspension of embodiment 1, in which the concentration of microbubbles per millilitre is between 10⁸ and 10¹⁰.

3. The injectable suspension of embodiment 1, in which the concentration of phospholipids is above 0.00013% wt.

4. The injectable suspension of any preceding embodiment, in which the liquid carrier further comprises water soluble poly- and oligo-saccharides, sugars and hydrophilic polymers such as polyethylene glycols as stabilizers.

5. The injectable suspension of any preceding embodiment, in which the phospholipids are at least partially in lamellar or laminar form and are selected from lecithins such as phosphatidic acid, phosphatidylcholine, phosphatidylethanolamine, phosphatidyl-serine, phosphatidylglycerol phosphatidylinositol, cardiolipin and sphingomyelin.

6. The injectable suspension of embodiment 4 or 5, further containing substances affecting the properties of phospholipids selected from phosphatidylglycerol, phosphatidic acid, dicetylphosphate, cholesterol, ergosterol, phytosterol, sitosterol, lanosterol, tocopherol, propylgallate, ascorbyl palmitate and butylated hydroxy-toluene.

7. The injectable suspension of embodiment 1, 2 or 3, in which the phospholipids are in the form of powders obtained by freeze-drying or spray-drying.

8. The injectable suspension of embodiment 1, containing about 10⁸-10⁹ microbubbles per millilitre with the microbubble size between 0.5-10 μm showing little or no variation under storage.

9. The injectable suspension of embodiment 1, in which the liquid carrier further comprises up to 50% by weight non-laminar surfactants selected from fatty acids, esters and ethers of fatty acids and alcohols with polyols such as polyalkylene glycols, polyalkylenated sugars and other carbohydrates, and polyalkylenated glycerol.

10. The injectable suspension of any preceding embodiment, in which the microbubbles are filled with SF₆, CF₄, or freons.

11. A method of making suspensions of gas filled microbubbles comprising selecting at least one film forming surfactant, converting the surfactant into a powder, contacting the powder with gas and admixing the powder surfactant with an aqueous liquid carrier to form said suspension, characterised by introducing the suspension into a container, forming a layer of the gas filled microbubbles in the upper part of the container, separating the layer of the microbubbles formed, and washing the microbubbles with an aqueous solution saturated with the microbubble gas.

12. The method of embodiment 11, in which prior to converting into the powder, the film forming surfactant is at least partially lamellarized.

13. The method of embodiment 12, in which prior to contacting with r gas the partially lamellarized surfactant is admixed with the aqueous liquid carrier

14. The method of embodiment 12 or 13, in which the liquid carrier further contains stabiliser compounds selected from hydrosoluble proteins, polypeptides, sugars, poly- and oligo-saccharides and hydrophilic polymers.

15. The method of embodiment 12, in which the conversion is effected by coating the surfactant onto particles of soluble or insoluble materials leaving the coated particles for a while under gas, and admixing the coated particles with an aqueous liquid carrier.

16. The method of embodiment 12, in which the conversion is effected by sonicating or homogenising under high pressure an aqueous solution of film forming lipids, this operation leading, at least partly, to the formation of liposomes.

17. The method of embodiment 16, in which prior to contacting of at least partially lamellarized surfactant with gas the liposome containing solution is freeze-dried.

18. The method of embodiments 16 and 17, in which the water solution of film forming lipids also contains viscosity enhancers or stabilisers selected from hydrophilic polymers and carbohydrates in weight ratio relative to the lipids comprised between 10:1 and 1000:1.

19. A method of preparation of a suspension of gas filled microbubbles comprising a film forming surfactant, a hydrophilic stabiliser and an aqueous liquid carrier, characterised by dissolving the film forming surfactant and the hydrophilic stabiliser in an organic solvent, freeze drying the solution to form a dry powder, contacting the powder with gas and admixing said powder with the aqueous carrier.

20. The method of embodiment 19, in which the hydrophilic stabiliser is polyethylene glycol, polyvinyl pyrrolidone, polyvinyl alcohol, glycolic acid, malic acid or maltol.

21. The method of embodiment 19 or 20, in which the organic solvent is tertiary butanol, 2-methyl-2-butanol or C₂Cl₄F₂.

22. A method of making an injectable suspension of gas-filled microbubbles according to embodiment 1, which comprises suspending laminarized phospholipids, and optionally other additives, in an aqueous carrier liquid, said phospholipids having been in contact with said gas prior or after being suspended, under conditions such that a concentration of said microbubbles sufficient to provide an echographic response is formed in the suspension, allowing a portion of said phospholipids to form a stabilization layer around said bubbles and thereafter depleting the carrier liquid of the excess of phospholipids not involved in microbubble stabilization.

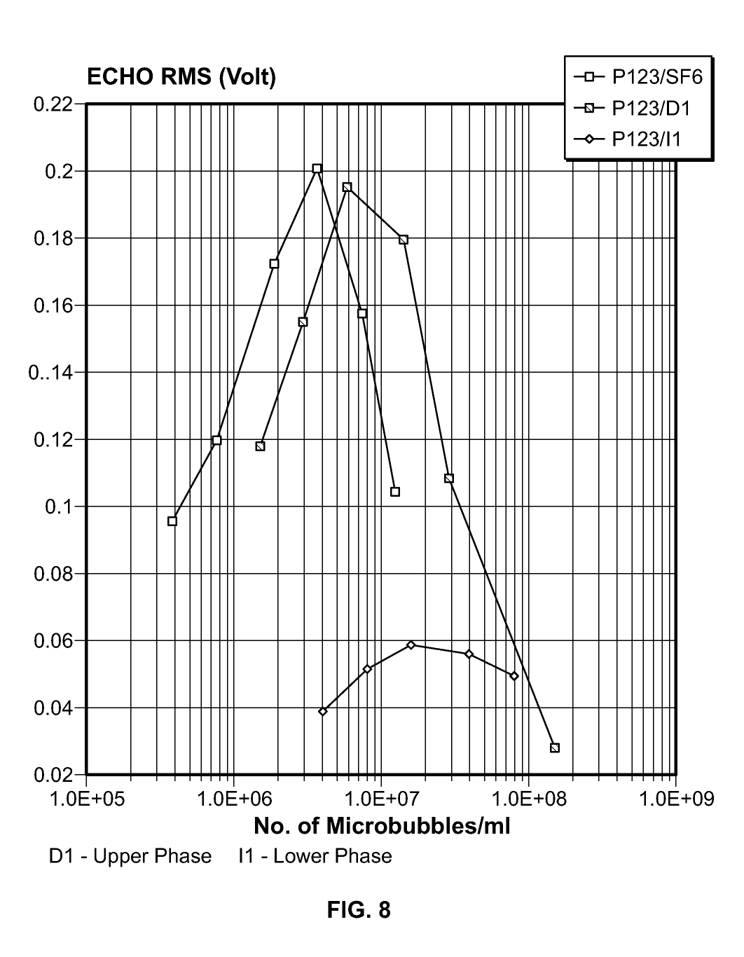

FIG. 8 is graphical presentation of echographic responses as a function of the microbubble concentration for a freshly prepared suspension according to the invention.

Suspensions and the method of making low phospholipid content suspensions of the invention will be further illustrated by the following examples:

### EXAMPLE 34

Multilamellar vesicles (MLVs) were prepared by dissolving 240 mg of diarachidoylphosphatidylcholine (DAPC, from Avanti Polar Lipids) and 10 mg of dipalmitoyl-phosphatidic acid (DPPA acid form, from Avanti Polar Lipids) in 50 ml of hexane/ethanol (8/2, v/v) then evaporating the solvents to dryness in a round-bottomed flask using a rotary evaporator. The residual lipid film was dried in a vacuum dessicator. After addition of water (5 ml), the suspension was incubated at 90° C. for 30 minutes under agitation. The resulting MLVs were extruded at 85° C. through a 0.3/m polycarbonate filter (Nucleopore®). 2.6 ml of the resulting MLV preparation were added to 47.4 ml of a 167 mg/ml solution of dextran 10,000 MW (Fluka) in water. The resulting solution was thoroughtly mixed, transferred in a 500 ml round-bottom flask, frozen at −45° C. and lyophilised under 0.1 Torr. Complete sublimation of the ice was obtained overnight. Thereafter, air pressure was restored in the evacuated container. Various amounts of the resulting powder were introduced in glass vials (see table 15) and the vials were closed with rubber stoppers. Vacuum was applied via a needle through the stopper and the air removed from vials. Upon evacuation of air the powder was exposed to sulfur hexafluoride gas, SF₆.

Bubble suspensions were obtained by injecting in each vial 10 ml of a 3% glycerol solution in a water (through the stopper) followed by gentle mixing. The resulting microbubble suspensions were counted using a hemacytometer. The mean bubble size (in volume) was 2.2 μ.

TABLE 15

| Dry weight | Phospholipid conc. | Concentration |
|---|---|---|
| (mg/ml) | (μg per ml) | (bubbles/ml) |
| 0.5 | 8 | 9.0 × 10⁶ |
| 1 | 16 | 1.3 × 10⁷ |
| 5 | 81 | 7.0 × 10⁷ |
| 10 | 161 | 1.4 × 10⁸ |

Preparations were injected to rabbits (via the jugular vein) as well as minipigs (via the ear vein) at a dose of 1 ml/5 kg. In vivo echographic measurements were performed using the Acuson XP128 ultrasound system (Acuson Corp. USA) and a 7 MHz sector transducer. The animals were anaesthetised and the transducer was positioned and then fixed in place on the left side of the chest providing a view of the right and left ventricles of the heart in the case of rabbit and a longitudinal four-chamber view in the case of the minipig. The preparation containing 0.5 mg/ml dry weight gave slight opacification of the right as well as the left ventricle in rabbits and in minipigs. The opacification, however, was superior with the 1, 5 and 10 mg/ml preparations.

### EXAMPLE 35

Lyophilisates were prepared as described in Example 34 with air (instead of SF₆) in the gas phase. The lyophilisates were then suspended in 0.9% saline (instead of a 3% glycerol solution). Similar bubble concentrations were obtained. However, after injection in the rabbit or the minipig the persistence of the effect was shorter e.g. 10-20 s instead of 120 s. Moreover, in the minipig the opacification of the left ventricle was poor even with the 10 mg/ml preparation.

### EXAMPLE 36

MLV liposomes were prepared as described in Example 34 using 240 mg of DAPC and 10 mg of DPPA (molar ratio 95:5). Two milliliters of this preparation were added to 20 ml of a polyethyleneglycol (PEG 2,000) solution (82.5 mg/ml). After mixing for 10 min at room temperature, the resulting solution was frozen during 5 min at 45° C. and lyophilised during 5 hours at 0.2 mbar. The powder obtained (1.6 g) was transferred into a glass vial equipped with a rubber stopper. The powder was exposed to SF₆ (as described in Example 34) and then dissolved in 20 ml of distilled water. The suspension obtained showed a bubble concentration of 5×10⁹ bubbles per ml with a median diameter in volume of 5.5 μm. This suspension was introduced into a 20 ml syringe, the syringe was closed and left in the horizontal position for 24 hours. A white layer of bubbles could be seen on the top of solution in the syringe. Most of the liquid phase (.about. 16-18 ml) was evacuated while the syringe was maintained in the horizontal position and an equivalent volume of fresh, SF₆-saturated, water was introduced. The syringe was then shaken for a while in order to homogenize the bubbles in the aqueous phase. A second decantation was performed under the same conditions after 8 hours followed by three further decantations performed in four hour intervals. The final bubble phase (batch P145) was suspended in 3 ml of distilled water. It contained 1.8×10⁹ bubbles per ml with a median diameter in volume of 6.2 μm. An aliquot of this suspension (2 ml) was lyophilised during 6 hours at 0.2 mbar. The resulting powder was dissolved in 0.2 ml of tetrahydrofuran/water (9/1 v/v) and the phospholipids present in this solution were analysed by HPLC using a light scattering detector. This solution contained 0.7 mg DAPC per ml thus corresponding to 3.9 μg of phospholipids per 10⁸ bubbles. A Coulter counter analysis of the actual bubble size distribution in batch P145 gave a total surface of 4.6×10⁷ μm² per 10⁸ bubbles. Assuming that one molecule of DAPC will occupy a surface of 50 Å², one can calculate that 1.3 μg of DAPC per 10⁸ bubbles would be necessary to form a monolayer of phospholipids around each bubble. The suspension P145 was than left at 4° C. and the concentration of gas bubbles measured on a regular basis. After 10 days, the product looked as good as after its preparation and still contained 1-1.2×10⁹ bubbles per ml. The exceptional stability was found very surprising considering the extremely low amount of phospholipids in the suspension.

The experiment described above was repeated on a second batch of microbubbles using a shorter decantation time in order to collect preferably larger bubbles (batch P132). The median diameter in volume obtained was 8.8 μm and the total surface determined with the Coulter counter was 22×10⁸ μm² per 10⁸ bubbles. The calculation showed that 6 μg DAPC for 10⁸ bubbles would be necessary to cover this bubble population with a monolayer of DAPC. The actual amount of DAPC determined by HPLC was 20 μg per 10⁸ bubbles. Taking into account the difficulty of obtaining precise estimates of the total surface of the bubble population, it appears that within the experimental error, the results obtained are consistent with coverage of the microbubbles with one phospholipid layer.

Echographic measurements performed with different washed bubble preparations showed that upon separation the lower phase gives a much weaker echographic signal than the upper phase or a freshly prepared sample. On a first sight this seemed normal as the white layer on the top of the syringe contained the majority of the gas microbubbles anyway. However, as shown in FIG. 8 the bubble count showed a surprisingly high microbubble population in the lower layer too. Only upon Coulter measurement it became apparent that the microbubbles had a size below 0.5 μm, which indicates that with small bubbles even when in high concentration, there is no adequate reflection of the ultrasound signal.

A four fold dilution of the preparation P132 in a 3% glycerol solution was injected in the minipig (0.2 ml/kg). The preparation of washed bubbles containing 2.5×10⁷ bubbles per ml and 5 μg of phospholipids per ml provided excellent opacification in the left and right ventricle with outstanding endocardial border delineation. Good opacification was also obtained by injecting to a minipig an aliquot of preparation P145 (diluted in 3% glycerol) corresponding to 0.2 μg of phospholipids per kg. Contrast was even detectable in the left ventricle after injection of 0.02 μg/kg. Furthermore, in the renal artery the existence of a contrast effect could be detected by pulsed Doppler at phospholipid doses as low as 0.005 μg/kg.

It follows that as long as the laminarized phospholipids are arranged in a single monolayer around the gas microbubbles the suspensions produced will have adequate stability. Thus providing an explanation for the present unexpected finding and demonstrating that the amount of phospholipids does not have to be greater than that required for formation of a monolayer around the microbubbles present in the suspension.

### EXAMPLE 37

A solution containing 48 mg of DAPC and 2 mg of DPPA in hexane/ethanol 8/2 (v/v) was prepared and the solvent evaporated to dryness (as described in Example 34). 5 mg of the resulting powder and 375 mg of polyethyleneglycol were dissolved in 5 g of tert-butanol at 60° C. The clear solution was then rapidly cooled to −45° C. and lyophilised. 80 mg of the lyophilisate was introduced in a glass vial and the powder exposed to SF₆ (see Example 1). A 3% glycerol solution (10 ml) was then introduced in the vial and the lyophilisate dissolved by gentle swirling. The resulting suspension had 1.5×10⁸ bubbles per ml with a median diameter (in volume) of 9.5 μm. This solution was injected to a rabbit providing outstanding views of the right and left ventricle. Even a ten fold dilution of this suspension showed strong contrast enhancement.

### EXAMPLE 38

The procedure of Example 37 was repeated except that the initial dissolution of the phospholipids in hexane/ethanol solution was omitted. In other words, crude phospholipids were dissolved, together with polyethylene glycol in tertiary butanol and the solution was freeze-dried; thereafter, the residue was suspended in water. Several phospholipids and combinations of phospholipids with other lipids were investigated in these experiments. In the results shown in table 16 the phospholipids were dissolved in a tertiary butanol solution containing 100 mg/ml of PEG 2,000. The residues obtained after freeze drying were saturated with SF₆ (see Example 34), then dissolved in distilled water at a concentration of 100 mg dry weight per ml.

TABLE 16

| | Conc. in | | |
|---|---|---|---|
| Lipid mixture | tert-butanol | Bubble conc. | Median diam. |
| (weight ratio) | (mg/ml) | (×10⁹/ml) | (μm) |
| DSPC | 2 | 1.3 | 10 |
| DAPC/DPPG (100/4) | 2 | 3.8 | 7 |
| DSPC/Chol (2/1) | 6 | 0.1 | 40 |
| DAPC/Plur F68 (2/1) | 6 | 0.9 | 15 |
| DAPC/Palm. ac. (60/1) | 2 | 0.6 | 11 |
| DAPC/DPPA (100/4) | 1 | 2.6 | 8 |
| DAPC/Chol/DPPA (8/1/1) | 8 | 1.2 | 19 |
| DAPC/DPPA (100/4)* | 5 | 2.4 | 18 |

Legend
DAPC = diarachidoylphosphatidyl choline
DSPC = distearoylphosphatidyl choline
DPPG = dipalmitoylphosphatidyl glycerol (acid form)
DPPA = dipalmitoylphosphatidic acid
Chol = cholesterol
Palm. ac. = palmitic acid
Plur F68 = Pluronic ® F-68
*In this experiment, CF₄ was used as gas instead of SF₆

In all cases the suspensions obtained showed high microbubble concentrations indicating that the initial conversion of phospholipids into liposomes was not necessary. These suspensions were diluted in 0.15 M NaCl and injected to minipigs as described in Example 3. In all cases outstanding opacification of the right and left ventricles as well as good delineation of the endocardial border were obtained at doses of 10-50 μg of lipids per kg body weight or less.

### EXAMPLE 39

PEG-2000 (2 g), DAPC (9.6 mg) and DPPA (0.4 mg) were dissolved in 20 ml of tertiary butanol and the solution was freeze dried overnight at 0.2 mbar. The powder obtained was exposed to SF₆ and then dissolved in 20 ml of distilled water. The suspension containing 1.4×10⁹ bubbles per ml (as determined by hemacytometry) was introduced into a 20 ml syringe, which was closed and left in horizontal position for 16 hours. A white layer of bubbles could be seen on top of the solution. The lower phase (16-18 ml) was discarded while maintaining the syringe horizontally. An equivalent volume of fresh SF₆-saturated distilled water was aspirated in the syringe and the bubbles were homogenised in the aqueous phase by agitation. Two different populations of microbubbles i.e. large-sized and medium-sized were obtained by repeated decantations over short periods of time, the large bubbles being collected after only 10-15 min of decantation and the medium sized bubbles being collected after 30-45 min. These decantations were repeated 10 times in order to obtain narrow bubble size distributions for the two types of populations and to eliminate all phospholipids which were not associated with the microbubbles. All phases containing large bubbles were pooled (“large-sized bubbles”). Similarly the fractions containing medium sized bubbles were combined (“medium-sized bubbles”). Aliquots of the two bubble populations were lyophilised and then analysed by HPLC in order to determine the amount of phospholipids present in each fraction. The large-sized bubble fraction contained 2.5×10⁷ bubbles per ml with a median diameter in number of 11.3 μm and 13.7 μg phospholipids per 10⁷ bubbles. This result is in excellent agreement with the theoretical amount, 11.5 μg per 10⁷ bubbles, calculated assuming a monolayer of phospholipids around each bubble and a surface of 50 Å per phospholipid molecule. The medium-sized bubble fraction contained 8.8×10⁸ bubbles per ml with a median diameter in number of 3.1 μm and 1.6 μg phospholipids per 10⁷ bubbles. The latter value is again in excellent agreement with the theoretical amount, 1.35 μg per 10⁷ bubbles. These results further indicate that the stability of the microbubble suspensions herein disclosed is most probably due to formation of phospholipid monolayers around the microbubbles.

Additional Stable Microbubbles Suspensions

As discussed above, while the microbubble suspensions of the invention may employ virtually any biocompatible and amphipathic compound capable of forming stable films in the presence of an aqueous phase and a gas, phospholipids are preferred. Phospholipids useful in the invention include: phosphatidylcholine (PC) with both saturated and unsaturated lipids; including phosphatidylcholine such as dioleylphosphatidylcholine; dimyristoylphosphatidylcholine (DMPC), dipentadecanoylphosphatidylcholine-, dilauroylphosphatidylcholine (DLPC); dipalmitoylphosphatidylcholine (DPPC); disteraoylphosphatidylcholine (DSPC); and diarachidonylphosphatid-ylcholine (DAPC); phosphatidylethanolamines (PE), such as dioleylphosphatidylethanolamine, dipaimitoylphosphatidylethanolamine (DPPE) and distearoylphosphatidylethanolamine (DSPE); phosphatidylserine (PS) such as dipalmitoyl phosphatidylserine (DPPS), disteraoylphosphatidylserine (DSPS); phosphatidylglycerols (PG), such as dipalmitoylphosphatidylglycerol (DPPG), distearoylphosphatidylglycerol (DSPG); and phosphatidylinositol. Saturated phospholipids are particularly preferred. Indeed, in a preferred embodiment disteraoylphosphatidylcholine (DSPC) and dipalmitoylphosphatidylglycerol (DPPG) are used.

As noted above, any physiologically acceptable gas may be present in the agents of the present invention. The term “gas” as used herein includes any substances (including mixtures) substantially in gaseous form at the normal human body (37° C.). The gas may comprise, for example, air; nitrogen; oxygen; CO₂; hydrogen, nitrous oxide; noble or inert gases such as helium, argon, xenon or krypton; fluorinated gases; and mixtures thereof, with fluorinated gases being preferred. Fluorinated gases include materials which contain at least one fluorine atom such as SF₆, freons (organic compounds containing one or more carbon atoms and fluorine, i.e. CF₄, C₂F₆, C₃F₈, C₄F₈, C₄F₁₀, CBrF₃, CCl₂F₂, C₂ClF₅ and CBrClF₂) and perfluorocarbons. The term perfluorocarbon refers to compounds containing only carbon and fluorine atoms and includes saturated, unsaturated, and cyclic perfluorocarbons such as perfluoroalkanes such as perfluoromethane, perfluoroethane, perfluoropropanes, perfluorobutanes (e.g. perfluoro-n-butane, optionally in admixture with other isomers such as perfluoro-isobutane), perfluoropentanes, perfluorohexanes and perfluoroheptanes; perfluoroalkenes such as perfluoropropene, perfluorobutenes (e.g. perfluorobut-2ene) and perfluorobutadiene; perfluoroalkynes such as perfluorobut-2-yne; and perfluorocycloalkanes such as perfluorocyclobutane, perfluoromethylcyclobutane, perfluorodimethylcyclob-utanes, perfluorotrimethylcyclobutanes, perfluorocyclopentane, perfluoromethylcyclopentane, perfluorodimethylcyclopentanes, perfluorocyclohexane, perfluoromethylcyclohexane and perfluorocycloheptane.). The saturated perfluorocarbons, which are usually preferred, have the formula C_{.n}Fₙ₊₂, where n is from 1 to 12, preferably from 2 to 10, most preferably from 3 to 8 and even more preferably from 3 to 6. Suitable perfluorocarbons include, for example, CF₄, C₂F₆, C₃F₈ C₄F₈, C₄F₁₀, C₅F₁₂, C₆F₁₂, C₇F₁₄, C₈F₁₈, and C₉F₂₀. In particularly preferred embodiments, SF₆ or perfluorocarbon freons selected from the group consisting of CF₄, C₂F₆, C₃F₈C₄F₈, and C₄F₁₀ are employed in the gas or gas mixture, with use of SF₆, C₃F₈ or C₄F₁o being particularly preferred. In a preferred embodiment the microbubbles of the invention contain SF₆.

As cited above the gas can be a mixture of the gases disclosed herein. In particular the following combinations are particularly preferred: a mixture of gases (A) and (B) in which, at least one of the gases (B), present in an amount of between 0.5-41% by vol., has a molecular weight greater than 80 daltons and (B) is is selected from the group consisting of SF₆, CF₄, C₂F₆, C₂F₈, C₃F₆, C₃F₈, C₄F₆, C₄F₈, C₄F₁₀, C₅F₁₂ and mixtures thereof and (A) is selected from the group consisting of air, oxygen, nitrogen, carbon dioxide and mixtures thereof, the balance of the mixture being gas A

In certain circumstances it may be desirable to include a precursor to a gaseous substance (e.g. a material that is capable of being converted to a gas in vivo). Preferably the gaseous precursor and the gas it produces are physiologically acceptable. The gaseous precursor may be pH-activated, photo-activated, temperature activated, etc. For example, certain perfluorocarbons may be used as temperature activated gaseous precursors. These perfluorocarbons, such as perfluoropentane, have a liquid/gas phase transition temperature above room temperature (or the temperature at which the agents are produced and/or stored) but below body temperature; thus, they undergo a phase shift and are converted to a gas within the human body.

As discussed above, the present invention also includes dry formulations which may be used to generate the injectable suspensions of the invention upon simple mixing with an aqueous carrier phase. The dry formulations will generally be in powder or in a cake form and are readily reconstitutable in a suitable aqueous liquid carrier, which is physiologically acceptable, sterile and injectable. Suitable liquid carriers are water, aqueous solutions such as saline (which may advantageously be balanced so that the final product for injection is not hypotonic), or solutions of one or more tonicity adjusting substances such as salts or sugars, sugar alcohols, glycols and other non-ionic polyol materials (eg. glucose, sucrose, sorbitol, mannitol, glycerol, polyethylene glycols, propylene glycols and the like). Reconstitution will generally require only minimal agitation such as may, for example, be provided by gentle hand-shaking. The size of the microbubbles so generated is consistently reproducible and in practice is independent of the amount of agitational energy applied.

The dry formulations will include one or more of the film forming surfactants discussed herein and may include one or more hydrophilic stabilizers and/or addditives . As discussed above, such hydrophilic stabilizers may include a polymer like PVP, PVA, PEG, etc. or a compound soluble both in the organic solvent and water and freeze-drying or spray-drying the solution. Further examples of the hydrophilic stabiliser compounds soluble in water and the organic solvent are malic acid, glycolic acid, maltol and the like. In a preferred embodiment, the hydrophilic stabilizer is polyethylene glycol (PEG) with a molecular weight from about 1000 to about 7500, with a molecular weight from about 2000 to about 5000 being preferred and PEG 4000 being most preferred. The additives may include compounds discussed herein and in certain cases the additives may act as opsonisation inhibitors delaying the uptake of the microbubbles from the vasculature by the reticuloendothelial system.

In practice injectable compositions prepared from the dry formulations should be as close to isotonic with blood as possible. Hence, before injection, small amounts of isotonic agents may also be added to the suspensions of the invention. The isotonic agents are physiological solutions commonly used in medicine and they comprise aqueous saline solution (0.9% NaCl), 2.6% glycerol solution, 5% dextrose solution, etc. Other excipients may optionally be present in the composition being dried or may be added on formulation for administration. Such excipients may for example include pH regulators, osmolality adjusters, viscosity enhancers, emulsifiers, bulking agents, etc. and may be used in conventional amounts.

The preferred dry formulations of ultrasound contrast agents of the present invention not only provide advantages for transport and storage due to the reduction in bulk relative to aqueous dispersions, but they also provide other advantages over freeze-dried products disclosed in the prior art. Specifically, freeze dried products of the prior art are not thermally stable in the range of ambient temperatures normally encountered during transportation and storage and as a result must be maintained in an environment in which the temperature is maintained at or below ambient (e.g. at 5 to 25° C.).

In contrast, the preferred dry formulations of the instant invention are thermally stable at all temperatures normally encountered during transportation and storage. Therefore, these dry formulations may be stored and transported without need of temperature control of the environment and in particular may be supplied to hospitals and physicians for on site formulation into an administrable dispersion without requiring such users to have special storage facilities. Lyophilized products according to the invention have proved to be storage stable for several months under ambient conditions. The microbubble dispersions generated upon reconstitution with an aqueous carrier liquid are stable for considerable lengths of time, e.g. up to at least 12 hours, permitting considerable flexibility as to when the dried product is reconstituted prior to injection.

These preferred dry formulations include an additive comprising one or more lipid compounds, which serve as a preserving agent, preventing significant alteration of the acoustic properties of the reconstituted aqueous suspension after storage of the dry formulation over time and at temperatures far exceeding ambient temperature.

This preserving agent is selected from fatty acids, phospholipids in acid form or a mixture thereof. Additionally, other lipid acids may be used as preserving agents, preferably those having a temperature of fusion greater than 40° C. (T_{f}>40° C.). While both saturated and unsaturated fatty acids may be used, the preserving agent is preferably a C12-C24 straight chain saturated fatty acid selected from lauric acid, myristic acid, palmitic acid, stearic acid, arachidic acid, behenic acid, lignoceric acid or a mixture thereof. More preferably, the preserving agent is a C14-C20 straight chain saturated fatty acid selected from myristic acid, palmitic acid, stearic acid, arachidic acid or a mixture thereof In a particularly preferred mode of the present invention, the preserving agent is palmitic acid.

When the preserving agent is chosen among the family of phospholipids in acid form, a saturated phospholipid in acid form selected from dimyristoylphosphatidic acid, dimyristoylphosphatidylglycer-ol, dimyristoylphosphatidylserine, dipalmitoylphosphatidic acid (DPPA), dipalmitoylphosphatidylglycerol (DPPG), dipalmitoylphosphatidylserine (DPPS), distearoylphosphatidic acid (DSPA), distearoylphosphatidylglycero-1 (DSPG), distearoylphosphatidylserine (or mixtures thereof) is preferred. Lyso forms of such charged phospholipids are also useful in accordance with the invention, the term “lyso” denoting phospholipids containing only one fatty acyl group. Such lyso forms of phospholipids may advantageously be used in acid form in admixture with phospholipids. One may obtain the acid form of the lipid by protonating it.

The preferred dry formulations of the invention are thermally stable at temperatures in excess of at least 20° C., preferably at least 22° C., 25° C. or 30° C. and especially preferably they are stable at at least 40° C., eg. up to 50° C. Thus, the dry formulation may be stored at a temperature of 20° C., 30° C. or even at 40° C. for a period of one or even two months or more and retain its acoustical properties upon reconstitution. The lipid preserving agents of the present invention prevent the alteration of the acoustic properties (echogenic response) after the reconstitution in an aqueous suspension of a dry powder stored for a period of at least one-month at 40° C. The preserving agent is present in the dry formulation at a concentration comprised between 1 and 50% by weight of the total amount of the phospholipid film forming surfactant, preferably between 5 and 25% by weight and even more preferably between 10 and 15% by weight of the phospholipid film forming surfactant. In a particularly preferred mode of the present invention, the preserving agent is palmitic acid at a concentration comprised between 10 and 15% by weight of the phospholipid film forming surfactant. As discussed above, after dispersion or reconstitution of the dry formulation in water or in an aqueous carrier liquid, the phospholipid film forming surfactants are present at a concentration in the carrier liquid below 0.01% by weight.

The lipid preserving agent according to the present invention is a constituent of the membrane and will incorporate into the mono-molecular layer (e.g. the monolayer) surrounding the bubble after reconstitution of the contrast agent in the liquid carrier.

The thermally stable dry formulations of the present invention may be prepared by selecting at least one film forming phospholipid surfactant, converting said phospholipid into a powder, and admixing the powdered phospholipid with one or more lipid preserving agents. The film forming surfactant mixture may be converted to a dry powder by, for example, dissolving the film forming surfactant (with or without the preserving agent) in an organic solvent and freeze drying or spray drying the solution to form a dry powder. Alternatively, the lipid preserving agent can be added to the film forming surfactant mixture prior to its conversion to a powder. Then the surfactant mixture including the preserving agent is converted into a powder.

Prior to reconstitution and optionally before or during packaging, the dry powder is contacted with the desired gas.

When contacted with the aqueous carrier the powdered phospholipids whose structure has been disrupted will form lamellarized or laminarized segments which will stabilise the microbubbles of the gas dispersed therein. The term lamellar or lamella or laminar form indicates that the surfactants are in the form of thin films or sheets involving one or more molecular layers. As described in WO-A-91/15244 (incorporated by reference herein in its entirety) conversion of film forming surfactants into lamellar form can easily be done by any liposome forming method for instance by high-pressure homogenisation or by sonication under acoustical or ultrasonic frequencies. In another embodiment, the suspensions of the present invention may also be prepared with phospholipids which were lamellarized or laminarized prior to their contacting with gas. Hence, contacting the phospholipids with gas may be carried out when the phospholipids are in a dry powder form or in the form of a dispersion of laminarized phospholipids in the aqueous carrier.

If the dry formulation has been stored under or otherwise contacted with the desired gas, reconstitution of the aqueous microbubble suspension of the invention is obtained by simple dissolution of the dry formulation containing the preserving agent without any violent agitation. In a preferred embodiment the film forming surfactant mixture containing the preserving agent is freeze-dried under reduced pressure, permitting the restoration of the pressure above the dried powders with one of the preferred physiologically acceptable gases (i.e SF₆, C₄F₁₀ or a mixture containing one of these gases). The dry formulation may then be stored under this desired gas until reconstitution with an aqueous carrier is desired.

If the dry formulation has not been contacted with the desired gas during storage or otherwise, reconstitution of the dry formulation is obtained by contacting the powder with the desired gas and admixing said powder with the aqueous carrier.

In a preferred embodiment, the film forming phospholipid surfactant(s) and a hydrophilic stabilizer are dissolved in an organic solvent along with a fatty acid preserving agent. The solution is frozen and lyophilized and then the air above the lyophilizate is replaced with the desired gas and the vials of dry formulation are sealed. An echogenic suspension of microbubbles is prepared by reconstituting the dry formulation with saline solution or another physiologically acceptable aqueous liquid carrier.

In one embodiment, the dry formulation is contacted with air or another gas and admixed with an aqueous liquid carrier in a closed container whereby a suspension of microbubbles will form. The suspension is allowed to stand for a while and a layer of gas filled microbubbles formed is left to rise to the top of the container. The lower part of the mother liquor is then removed and the supernatant layer of microbubbles washed with an aqueous solution saturated with the gas used in preparation of the microbubbles. This washing can be repeated several times until substantially all unused or free surfactant molecules are removed. Unused or free molecules mean all surfactant molecules that do not participate in formation of the stabilising monomolecular layer around the gas microbubbles.

In a variant of the preceding embodiment, the dry formulation may be admixed with the aqueous liquid carrier prior to contacting with gas.

As discussed above, the volume and concentrations of the reconstitution liquid may desirably be balanced to make the resulting ready-to-use formulations substantially isotonic. Hence the volume and concentration of reconstitution fluid chosen will be dependent on the type and amount of stabilizer (and/or other bulking agents) present in the freeze-dried product.

The reconstituted contrast agents according to the invention also surprisingly enhance the ability of the microbubbles to retain the fluorinated gases and gas precursors commonly used in the ultrasound contrast agents of the invention.

It will be appreciated that kits can be prepared for use in making the microbubble preparations of the present invention. These kits can include a container containing all of the sterile dry components of the present invention and enclosing the preferred gas or gas mixture in one chamber. The sterile aqueous liquid may be contained in a second chamber of the same container. In one embodiment, the container is a conventional septum-sealed vial. In another, it has a means for directing or permitting application of sufficient bubble forming energy into the contents of the container.

Alternatively, a two container kit may be used in which the dry formulation of the invention may be included in one container together with the desired gas and the sterile aqueous carrier liquid may be included in a separate container in such away that it may be added to the first container under sterile conditions.

The invention has been described above with reference to dry formulations and microbubble suspensions for use as ultrasound contrast agents. However it is also applicable to use of such formulations and suspensions as contrast agents for other diagnostic imaging modalities (e.g. MRI, X-ray, SPECT, PET, magnetic imaging etc.).

As discussed above, the invention comprises in one aspect an injectable aqueous suspension of gas filled microbubbles usable as imaging contrast agent in ultrasonic echography comprising a concentration of phospholipids or other film forming surfactants of below about 0.01% by weight and optionally, additives such as a lipid preserving agent and a hydrophilic stabilizer.

Viewed from another aspect, the invention provides a dry formulation of an ultrasound contrast agent comprising one or more film forming surfactants which may be reconstituted in an aqueous carrier to yield an injectable, echogenic suspension of microbubbles containing less than 0.01% surfactants by weight. In a preferred embodiment the dry formulation further comprises an additive which serves as a preserving agent, permitting the dry formulation to be stored over time and at temperatures considerably higher than ambient temperature while preserving the echogenicity of the reconstituted suspensions. In a particularly preferred embodiment the dry formulation further comprises a hydrophilic stabilizer.

Viewed from a further aspect the invention provides methods of making such dry formulations.

Viewed from a still further aspect the invention provides a method of preparation of a reconstituted suspension starting from the dry formulations disclosed above, characterised by dissolving the phospholipid film forming surfactant, preferably with the preserving agent, in an organic solvent, freeze drying or spray drying the solution to form a dry powder, contacting the powder with gas and admixing said powder with the aqueous liquid carrier.

Viewed from a still further aspect the invention provides the use of one or more lipidic acid as preserving agent (having the ability to prevent the alteration or a significant alteration of the acoustic properties of the reconstituted aqueous suspension after the storage of the dry formulation for a period of at least one month at 40° C.) for the manufacture of an injectable aqueous suspension of gas filled microbubbles for use in diagnosis involving diagnostic ultrasound imaging.

Viewed from a yet still further aspect, the invention provides an injectable reconstituted suspension of gas filled microbubbles usable as ultrasound contrast agents comprising a phospholipid film forming surfactant present at a concentration below 0.01% by weight and an aqueous liquid carrier, characterised in containing one or more lipid preserving agent which, prevents the alteration or a significant alteration of the acoustic properties of the reconstituted aqueous suspension after the storage of the suspension in a dry form for a period of at least one month at 40° C. and optionally a hydrophilic stabilizer or other additive.

Viewed from a further aspect the invention provides a method for imaging an object or body or a region of a body, comprising the steps of: introducing into said object or body or body part or body cavity the injectable reconstituted microbubble suspension as defined above; and then imaging at least a portion of said body by ultrasound or another method of diagnostic imaging (e.g. magnetic resonance imaging etc). According to this method, said body is a vertebrate and said suspension is introduced into the vasculature or body cavity of said vertebrate.

The foregoing description will be more fully understood with reference to the following Examples. These Examples, are, however, exemplary of methods of practising the present invention and are not intended to limit the scope of the invention.

### EXAMPLE 40 (COMPARATIVE)

### Preparation with DPPA-Na

A solution containing 1 g of DPPC (dipalmitoylphosphatidylcholine) and 100 mg of DPPA-Na (both purchased from Lipoid, Switzerland) was prepared with 50 ml of hexane/isopropanol 8/2 (v/v; Fluka, Switzerland). The solvent was evaporated to dryness. 100 mg of the resulting powder and 10 g of Macrogol 4000 (Clarian, Germany) (PEG 4000) were dissolved in 60 g of tert-butanol at 60° C. to obtain a clear solution. The solution was aliquoted into 100 glass vials of 100 ml and rapidly frozen at −45° C. and lyophilized. The resulting lyophilisate was exposed to SF₆ by replacing air and sealed with stopper within the freeze-dryer (Christ®). The vials of the lyophilisate sample were then stored in ovens at 5, 25, 40° C. and 50° C. in order to perform one month-stability test. To evaluate lyophilisate quality (microbubble. formation), the lyophilisate sample was reconstituted with 10 ml saline solution (0.9%-NaCl). Bubble echogenicity (backscatter coefficient measured at 7 MHz, see M. Schneider, Echocardiography: A Jrnal. of CV Ultrasound & Allied Tech., Vol. 16 No. 7, part 2, 1999 p 743-746) and bubble concentration (Coulter Counter) of the suspension were determined. The results of the stability test are summarized below in table 17

TABLE 17

| | | | Bubble |
|---|---|---|---|
| | Backscatter | Bubble conc | Volume |
| Storage | 10²/(sr · cm) | (10⁸/ml) | (μl/ml) |
| After preparation | 2.8 | 2.3 | 3.4 |
| 1 month at 5° C. | 2.9 | 2.4 | 3.5 |
| 1 month at 25° C. | 2.4 | 1.7 | 3.0 |
| 1 month at 40° C. | 1.5 | 1.2 | 2.2 |
| 1 month at 50° C. | 0.9 | 0.8 | 1.7 |

These results clearly indicate that the bubble sample concentration and echogenicity decrease with the storage. The higher the temperature the lower is the quality of the bubble sample.

### EXAMPLE 41

### Addition of DPPA-H to Improve the Lyophilisate Stability

(1) Preparation of DPPA-H

Two grams of DPPA-Na were disssolved in a mixed solvent containing chloroform (50 ml), methanol (60 ml) and distilled water (50 ml). After 10 minutes agitation (magnetic stirring), 35 ml of HCl solution (0.1 N) was added to the solution and the resulting solution was again agitated for 1 hour at room temperature. Then the solution was poured into a conical ampoule to separate the organic (chloroform) and aqueous phases (H2O+methanol). DPPA-H was finally obtained by eliminating the chloroform and residual solvent by evaporation under reduced pressure without heating and finally by lyophilisation.

(2) Stability of Lyophilisate Prepared with DPPA-H

Example 40 was repeated except that DPPA-Na (sodium form) was replaced by DPPA-H (acid form). The turbidity and Coulter Counter analyses were performed on reconstituted lyophilized samples (Oust after the preparation and 1 month later). It was very surprising to find that the stability of the lyophilized sample containing DPPA-H was considerably improved as one can note from Table 18.

TABLE 18

| | | | Bubble |
|---|---|---|---|
| | Backscatter | Bubble conc | Volume |
| Storage | 10²/(sr · cm) | (10⁸/ml) | (μl/ml) |
| After preparation | 3.4 | 2.5 | 3.7 |
| 1 month at 5° C. | 3.6 | 2.6 | 3.8 |
| 1 month at 25° C. | 3.4 | 2.4 | 3.7 |
| 1 month at 40° C. | 3.5 | 2.7 | 3.6 |
| 1 month at 50° C. | 2.7 | 2.1 | 3.3 |

### EXAMPLE 42

### Addition of DPPG-H to the Lyophilized Preparation

DPPG-H was prepared using the protocol described above for DPPA-H. 100 mg of DSPC, 100 mg of DPPG-Na and 9.8 g of Macrogol 4000 were dissolved in 80 g of tert-butanol under reflux (82° C.). Then the resulting clear solution was equitably divided into two parts in glass bottles. In one solution, 10 mg of DPPG-H was added. After complete dissolution, both two solution samples were frozen and lyophilized. 100 mg of each lyophilisate were placed in glass vials and exposed to gas SF₆. The SF₆ containing lyophilisates were stored at different temperatures for 1 month. The results of the stability test showed that the formulation containing DPPG-H was much more stable than the one, which did not contain DPPG-H.

### EXAMPLE 43

### Addition of Palmitic Acid to the Lyophilized Preparation

TABLE 19

| | Without palmitic | |
|---|---|---|
| Storage | acid | With palmitic acid |
| After preparation | 3.8 | 3.9 |
| 1 month at 5° C. | 3.7 | 3.8 |
| 1 month at 25° C. | 2.9 | 3.7 |
| 1 month at 30° C. | 2.2 | 3.7 |
| 1 month at 35° C. | 1.5 | 3.6 |
| 1 month at 40° C. | 1.4 | 3.7 |
| 1 month at 50° C. | 0.5 | 2.8 |

The results indicate that addition of a tiny quantity of palmitic acid (0.2% by weight of dried lyophilisate) improved considerably the shelf life of the lyophilisate during storage.

### EXAMPLE 44

### Addition of Various Fatty Acids

The procedure of Example 42 was repeated except that DPPG-H was replaced by 0.2% of one of several negatively charged phospholipids (in acid form) or fatty acids (lauric, myristic, palmitic and stearic acids). The lyophilized samples were exposed to SF₆ and C₄F₁₀ gases, then stored 1 month at 40° C. Stability test was performed as before. Table 20 shows the results (backscatter coefficient %).

TABLE 20

| Storage | SF₆ | C₄F₁₀ |
|---|---|---|
| Without fatty acid | 37 | 41 |
| DSPA-H | 101 | 96 |
| DSPG-H | 99 | 103 |
| DPPS-H | 103 | 98 |
| Lauric acid | 98 | 100 |
| Myristic acid | 101 | 99 |
| Palmitic acid | 102 | 98 |
| Stearic acid | 105 | 102 |

These data show that the negatively charged phospholipids in acid form and fatty acids in general can improve the stability of the microbubbles forming lyophilisate during storage.

### EXAMPLE 45

### Influence of the Amount of DPPG-H

The procedure of Example 42 was repeated with different amounts of DPPG-H (from 0 to 25% of the total lipids used for the preparation). The results of absorbance measurements (see the description in U.S. Pat. No. 5,578,292 incorporated by reference herein in its entirety), obtained from reconstituted SF₆ filled-lyophilisate at t0 (after preparation) and after one month of storage at 40° C. (t1), are set forth in Table 21.

TABLE 21

Absorbance
measurements
(700 nm)

| % DPPG-H | t0 | t1 | stability % |
|---|---|---|---|
| 0 | 0.24 | 0.13 | 54 |
| 2 | 0.25 | 0.17 | 68 |
| 5 | 0.25 | 0.21 | 84 |
| 10 | 0.27 | 0.28 | 103 |
| 15 | 0.30 | 0.29 | 97 |
| 20 | 0.23 | 0.20 | 87 |
| 25 | 0.24 | 0.19 | 79 |

### EXAMPLE 46

### Influence of the Amount Palmitic Acid)

The procedure of Example 43 was performed with different amounts of palmitic acid (from 0 to 25% of the total lipids used for the preparation). The results of Coulter measurements (bubble concentration) obtained from reconstituted SF₆ filled-lyophilisate at t0 and after one month of storage at 40° C. (t1), are gathered in Table 22.

TABLE 22

(bubble conc.
10⁸/ml))

| % Palm.acid | t0 | t1 | stability % |
|---|---|---|---|
| 0 | 2.5 | 1.2 | 48 |
| 2 | 2.6 | 1.6 | 62 |
| 5 | 2.5 | 1.9 | 76 |
| 10 | 2.6 | 2.5 | 96 |
| 15 | 2.3 | 2.2 | 96 |
| 20 | 1.5 | 1.3 | 87 |
| 25 | 0.6 | 0.5 | 83 |

The invention described herein can be further elucidated by the description of the following representative (but not limiting) embodiments:

1. An ultrasound contrast agent comprising an aqueous suspension of gas filled microbubbles comprising a saturated phospholipid, a fatty acid, a hydrophilic stabilizer, and SF₆, wherein the amount of the saturated phospholipid in the suspension is less than about 0.01% by weight.

2. The ultrasound contrast agent of embodiment 1, wherein the fatty acid is present in an amount between 1% and 50% by weight of the amount of the saturated phospholipid.

3. The ultrasound contrast agent of embodiment 1, wherein the fatty acid is present in an amount between 5% and 25% by weight of the amount of the saturated phospholipid.

4. The ultrasound contrast agent of embodiment 1, wherein the fatty acid is present in an amount between 10% and 15% by weight of the amount of the saturated phospholipid.

5. The ultrasound contrast agent of embodiment 1, wherein the fatty acid is a C₁₂-C.₂₄ straight chain saturated fatty acid selected from the group consisting of lauric acid, myristic acid, palmitic acid, stearic acid, arachidic acid, behenic acid, lignoceric acid and mixtures thereof.

6. The ultrasound contrast agent of embodiment 1, wherein the fatty acid comprises palmitic acid in an amount between 10% and 15% by weight of the amount of the saturated phospholipid.

7. The ultrasound contrast agent of embodiment 1, wherein the saturated phospholipid is selected from the group consisting of dimyristoylphosphatidic acid, dimyristoylphosphatidylglycerol, dimyristoylphosphatidylserine, dipalmitoylphosphatidic acid, dipalmitoylphosphatidylglycerol, dipalmitoylphosphatidylserine, distearoylphosphatidic acid, distearoylphosphatidylglycerol, distearoylphosphatidylserine and mixtures thereof.

8. The ultrasound contrast agent of embodiment 1, wherein the saturated phospholipid comprises distearoylphosphatidylcholine (DSPC) and dipalmitoylphosphatidylglycerol (DPPG).

9. The ultrasound contrast agent of any one of embodiments 1, 6 or 8 wherein the hydrophilic stabilizer comprises PEG 4000.

10. The ultrasound contrast agent of embodiment 1, wherein the saturated phospholipid comprises distearoylphosphatidylcholine (DSPC) and dipalmitoylphosphatidylglycerol (DPPG), the fatty acid comprises palmitic acid in an amount between 10 and 15% by weight of the amount of the saturated phospholipid, and the hydrophilic stabilizer comprises PEG 4000.

11. A method of imaging a region of a body comprising: (a) administering to the body an aqueous suspension of gas filled microbubbles comprising a saturated phospholipid, a fatty acid, a hydrophilic stabilizer, and SF₆, wherein the amount of the saturated phospholipid in the suspension is less than 0.01% by weight; and (b) imaging the body.

12. The method of imaging of embodiment 11, wherein the fatty acid is present in an amount between 1% and 50% by weight of the amount of the saturated phospholipid.

13. The method of imaging of embodiment 11, wherein the fatty acid is present in an amount between 5% and 25% by weight of the amount of the saturated phospholipid.

14. The method of imaging of embodiment 11, wherein the fatty acid is present in an amount between 10% and 15% by weight of the amount of the saturated phospholipid.

15. The method of imaging of embodiment 11, wherein the fatty acid is a C₁₂-C₂₄ straight chain saturated fatty acid selected from the group consisting of lauric acid, myristic acid, palmitic acid, stearic acid, arachidic acid, behenic acid, lignoceric acid and mixtures thereof.

16. The method of imaging of embodiment 11, wherein the fatty acid comprises palmitic acid in an amount between 10% and 15% by weight of the amount of the saturated phospholipid.

17. The method of imaging of embodiment 11, wherein the saturated phospholipid is selected from the group consisting of dimyristoylphosphatidic acid, dimyristoylphosphatidylglycerol, dimyristoylphosphatidylserine, dipalmitoylphosphatidic acid, dipalmitoylphosphatidylglycerol, dipalmitoylphosphatidylserine, distearoylphosphatidic acid, distearoylphosphatidylglycerol, distearoylphosphatidylserine and mixtures thereof.

18. The method of imaging of embodiment 11, wherein the saturated phospholipid comprises distearoylphosphatidylcholine (DSPC) and dipalmitoylphosphatidylglycerol (DPPG).

19. The method of imaging of any one of embodiments 11, 16 or 18, wherein the hydrophilic stabilizer comprises PEG 4000.

20. The method of imaging of embodiment 11, wherein the saturated phospholipid comprises distearoylphosphatidylcholine (DSPC) and dipalmitoylphosphatidylglycerol (DPPG), the fatty acid comprises palmitic acid in an amount between 10% and 15% by weight of the amount of the saturated phospholipid, and the hydrophilic stabilizer comprises PEG 4000.

21. The method of imaging of embodiment 11, wherein the body is a vertebrate and the suspension is administered to the vasculature or body cavity of the vertebrate.

22. A dry formulation of an ultrasound contrast agent comprising a saturated phospholipid, a fatty acid, and a hydrophilic stabilizer, wherein upon dissolution in an aqueous carrier liquid, the dry formulation will form a suspension of microbubbles comprising SF₆ in which the amount of saturated phospholipid in the suspension is less than about 0.01% by weight.

23. The dry formulation of embodiment 22, wherein the fatty acid is present in an amount of between 1% and 50% by weight of the amount of the saturated phospholipid.

24. The dry formulation of embodiment 22, wherein the fatty acid is present in an amount of between 5% and 25% by weight of the amount of the saturated phospholipid.

25. The dry formulation of embodiment 22, wherein the fatty acid is present in an amount of between 10% and 15% by weight of the amount of the saturated phospholipid.

26. The dry formulation of embodiment 22, wherein the fatty acid is a C₁₂-C₂₄ straight chain saturated fatty acid selected from the group consisting of lauric acid, myristic acid, palmitic acid, stearic acid, arachidic acid, behenic acid, lignoceric acid and mixtures thereof.

27. The dry formulation of embodiment 22, wherein the fatty acid comprises palmitic acid in an amount of between 10% and 15% by weight of the amount of the saturated phospholipid.

28. The dry formulation of embodiment 22, wherein the saturated phospholipid is selected from the group consisting of dimyristoylphosphatidic acid, dimyristoylphosphatidylglycerol, dimyristoylphosphatidylserine, dipalmitoylphosphatidic acid, dipalmitoylphosphatidylglycerol, dipalmitoylphosphatidylserine, distearoylphosphatidic acid, distearoylphosphatidylglycerol, distearoylphosphatidylserine and mixtures thereof.

29. The dry formulation of embodiment 22, wherein the saturated phospholipid comprises distearoylphosphatidylcholine (DSPC) and dipalmitoylphosphatidylglycerol (DPPG).

30. The dry formulation of any one of embodiments 22, 27 or 29, wherein the hydrophilic stabilizer comprises PEG 4000.

31. The dry formulation of embodiment 22, wherein the saturated phospholipid comprises distearoylphosphatidylcholine (DSPC) and dipalmitoylphosphatidylglycerol (DPPG), the fatty acid comprises palmitic acid in an amount of between 10% and 15% by weight of the amount of the saturated phospholipid, and the hydrophilic stabilizer comprises PEG 4000.

32. A method of using the dry formulation of any one of embodiments 22 to 29 or 31 for the preparation of an ultrasound contrast agent comprising forming a suspension of gas filled microbubbles with the dry formulation.

33. A method of preparing an ultrasound contrast agent comprising reconstituting the dry formulation of any one of embodiments 22 to 29 or 31 in an aqueous carrier liquid to form a suspension of gas filled microbubbles.

34. A method of imaging a region of a body comprising: (a) reconstituting the dry formulation of any one of embodiments 22 to 29 or 31 in an aqueous carrier liquid to form a suspension of gas filled microbubbles; (b) administering the suspension of gas filled microbubbles to the body; and (c) imaging the body.

35. A method of preparing a contrast agent comprising an aqueous suspension of gas filled microbubbles comprising SF₆ and saturated phospholipid, wherein the amount of saturated phospholipid in the suspension is less than about 0.01% by weight, the method comprising: (a) dissolving at least one saturated phospholipid, a fatty acid, and a hydrophilic stabilizer in an organic solvent to form a solution; (b) freeze drying or spray drying the solution to form a dried powder; (c) contacting the dried powder with SF₆; and (d) mixing the freeze dried or spray dried powder with an aqueous carrier phase.

36. A method of preparing a dry formulation of an ultrasound contrast agent, wherein upon dissolution in an aqueous carrier liquid, the dry formulation will form a suspension of microbubbles comprising SF₆ and saturated phospholipid, wherein the amount of the saturated phospholipid in the suspension is less than about 0.01% by weight, the method comprising: (a) dissolving at least one saturated phospholipid, a fatty acid, and a hydrophilic stabilizer in an organic solvent to form a solution; (b) freeze drying or spray drying the solution to form a dried powder; and (c) contacting the dried powder with SF₆.

37. A method of preparing a dry formulation of an ultrasound contrast agent, wherein upon dissolution in an aqueous carrier liquid, the dry formulation will form a suspension of microbubbles comprising SF₆ and saturated phospholipid, wherein the amount of the saturated phospholipid in the suspension is less than about 0.01% by weight, the method comprising: (a) dissolving at least one saturated phospholipid and a hydrophilic stabilizer in an organic solvent to form a solution; (b) freeze drying or spray drying the solution to form a dried powder; (c) mixing the dried powder with a fatty acid to form a mixture; and (d) contacting the mixture with SF₆.

38. An ultrasound contrast agent comprising an aqueous suspension of gas filled microbubbles comprising a saturated phospholipid, a preserving agent, SF₆, and a hydrophilic stabilizer, wherein the amount of saturated phospholipid in the suspension is less than about 0.01% by weight, and the preserving agent comprises a fatty acid.

39. The ultrasound contrast agent of embodiment 38, wherein the preserving agent is present in an amount between 1% and 50% by weight of the amount of the saturated phospholipid.

40. The ultrasound contrast agent of embodiment 38, wherein the preserving agent is present in an amount between 5% and 25% by weight of the amount of the saturated phospholipid.

41. The ultrasound contrast agent of embodiment 38, wherein the preserving agent is present in an amount between 10% and 15% by weight of the amount of the saturated phospholipid.

42. The ultrasound contrast agent of embodiment 38, wherein the preserving agent is a C₁₂-C₂₄ straight chain saturated fatty acid selected from the group consisting of lauric acid, myristic acid, palmitic acid, stearic acid, arachidic acid, behenic acid, lignoceric acid and mixtures thereof.

43. The ultrasound contrast agent of embodiment 38, wherein the preserving agent comprises palmitic acid in an amount between 10% and 15% by weight of the amount of the saturated phospholipid.

44. The ultrasound contrast agent of embodiment 38, wherein the saturated phospholipid is selected from the group consisting of dimyristoylphosphatidic acid, dimyristoylphosphatidylglycerol, dimyristoylphosphatidylserine, dipalmitoylphosphatidic acid, dipalmitoylphosphatidylglycerol, dipalmitoylphosphatidylserine, distearoylphosphatidic acid, distearoylphosphatidylglycerol, distearoylphosphatidylserine and mixtures thereof.

45. The ultrasound contrast agent of embodiment 38, wherein the saturated phospholipid comprises distearoylphosphatidylcholine (DSPC) and dipalmitoylphosphatidylglycerol (DPPG).

46. The ultrasound contrast agent of any one of embodiments 38, 43 or 45, wherein the hydrophilic stabilizer comprises PEG 4000.

47. The ultrasound contrast agent of embodiment 38, wherein the saturated phospholipid comprises distearoylphosphatidylcholine (DSPC) and dipalmitoylphosphatidylglycerol (DPPG), the preserving agent comprises palmitic in an amount between 10% and 15% by weight of the amount of the saturated phospholipid, and the hydrophilic stabilizer comprises PEG 4000.

48. A dry formulation of an ultrasound contrast agent comprising a saturated phospholipid, a preserving agent, and a hydrophilic stabilizer, wherein the preserving agent comprises a fatty acid, and upon dissolution in an aqueous carrier liquid, the dry formulation will form a suspension of microbubbles comprising SF₆ in which the amount of saturated phospholipid in the suspension is less than about 0.01% by weight.

49. The dry formulation of embodiment 48, wherein the preserving agent is present in an amount between 1% and 50% by weight of the amount of the saturated phospholipid.

50. The dry formulation of embodiment 48, wherein the preserving agent is present in an amount between 5% and 25% by weight of the amount of the saturated phospholipid.

51. The dry formulation of embodiment 48, wherein the preserving agent is present in an amount between 10% and 15% by weight of the amount of the saturated saturated phospholipid.

52. The dry formulation of embodiment 48, wherein the preserving agent is a C₁₂-C₂₄straight chain saturated fatty acid selected from the group consisting of lauric acid, myristic acid, palmitic acid, stearic acid, arachidic acid, behenic acid, lignoceric acid and mixtures thereof.

53. The dry formulation of embodiment 48, wherein the preserving agent comprises palmitic acid in an amount between 10% and 15% by weight of the amount of the saturated phospholipid.

54. The dry formulation of embodiment 48, wherein the saturated phospholipid is selected from the group consisting of dimyristoylphosphatidic acid, dimyristoylphosphatidylglycerol, dimyristoylphosphatidylserine, dipalmitoylphosphatidic acid, dipalmitoylphosphatidylglycerol, dipalmitoylphosphatidylserine, distearoylphosphatidic acid, distearoylphosphatidylglycerol, distearoylphosphatidylserine and mixtures thereof.

55. The dry formulation of embodiment 48, wherein the saturated phospholipid comprises distearoylphosphatidylcholine (DSPC) and dipalmitoylphosphatidylglycerol (DPPG).

56. The dry formulation of any one of embodiments 48, 53 or 55, wherein the hydrophilic stabilizer comprises PEG 4000.

57. The dry formulation of embodiment 48, wherein the saturated phospholipid comprises distearoylphosphatidylcholine (DSPC) and dipalmitoylphosphatidylglycerol (DPPG), the preserving agent comprises palmitic acid in an amount between 10% and 15% by weight of the amount of the saturated phospholipid, and the hydrophilic stabilizer comprises PEG 4000.

58. A method of using the dry formulation of any one of embodiments 48 to 55 or 57 for the preparation of an ultrasound contrast agent comprising forming a suspension of gas filled microbubbles from the dry formulation.

59. A method of preparing an ultrasound contrast agent comprising reconstituting the dry formulation of any one of embodiments 48 to 55 or 57 in an aqueous carrier liquid to form a suspension of gas filled microbubbles.

60. A method of imaging a region of a body comprising: (a) reconstituting the dry formulation of any one of embodiments 48 to 55 or 57 in an aqueous carrier liquid to form a suspension of gas filled microbubbles; (b) administering the suspension of gas filled microbubbles to the body; and (c) imaging the body.

1. A composition suitable for injection into the bloodstream and body cavities of living beings, comprising a suspension of stabilized air or gas microbubbles in a physiologically acceptable aqueous carrier phase having one or more dissolved or dispersed surfactants, at least one of said surfactants being a film forming saturated phospholipid present in the composition at least partially in lamellar or laminar form. wherein at least one of said surfactants comprise, bound thereto, bioactive species designed for specific targeting purposes.

2. A composition suitable for injection into the bloodstream and body cavities of living beings, comprising a suspension of air or gas microbubbles in a physiologically acceptable aqueous carrier phase stabilized having one or more dissolved or dispersed surfactants, at least one of said surfactants being a film forming saturated phospholipid present in the composition at least partially in lamellar or laminar form, said surfactants forming a surfactant layer stabilizing said microbubbles, wherein monoclonal antibodies tailored by genetic engineering, antibody fragments or polypeptides designed to mimic antibodies, bioadhesive polymers, lectins or other site-recognizing molecules are bound to the surfactant layer.

3. The composition of any one of claims 1 or 2, wherein the suspension contains at least 10⁷ microbubbles per milliliter and wherein the concentration of the phospholipid(s) in the liquid carrier is below 0.01% by weight.

4. The composition of claim 1, in which the concentration of microbubbles per milliliter is between 10⁸ and 10¹⁰.

5. The composition of claim 3, in which the concentration of phospholipids is above 0.00013% wt.

6. The composition of claim 1, in which the liquid carrier further comprises a stabilizer selected from the group consisting of water soluble poly- and oligosaccharides, sugars and hydrophilic polymers.

7. The composition to claim 6, wherein a hydrophilic polymer is a polyethylene glycol.

8. The composition of claim 1, in which the phosophilipid(s) are selected from the group consisting of phosphatidic acid, phosphatidylcholine, phosphatidylethanolamine, phosphatidylserine, phosphatidylglycerol, phosphatidylinositol, cardiolipin and sphingomyelin.

9. The composition of claim 1, further containing a substance affecting the properties of phospholipid selected from the group consisting of dicetylphosphate, cholesterol, ergosterol, phytosterol, sitosterol, lanosterol, tocopherol, propylgallate, ascorbyl palmitate and butylated hydroxytoluene.

10. The composition of claim 1, in which the phospholipid is in the form of powders obtained by freeze-drying or spray-drying.

11. The composition of claim 3, containing about 10⁸-10⁹ microbubbles per milliliter with the microbubble size between 0.5-10 mm showing little or no variation under storage.

12. The composition of claim 1, in which the liquid carrier further comprises up to 50% by weight non-laminar surfactants selected from the group consisting of fatty acids, esters and ethers of fatty acids and alcohols with polyols such as polyalkalene glycols, polyalkylenated sugars and other carbohydrates, and polyalkylenated glycerol.

13. The composition of claim 3, in which the microbubbles are filled with SF₆, CF₄, freons or air.

14. The composition of claim 1 wherein microbubbles are filled with a freon gas selected from CF₄, CBrF₃, C₄F₈, CClF₃, CCl₂ F₂, C₂F₆, C₂ClF₅, CBrClF₂, C₂Cl₂F₄, CBr₂F₂ and C₄ F₁₀.

15. The composition of claim 3 wherein the microbubbles are filled with a gas mixture of at least two biocompatible gases A and B in which at least one gas (B) present in an amount of between 0.5-41% by vol. has a molecular weight greater than 80 daltons and solubility in water below 0.0283 ml per ml of water at standard conditions, the balance of the mixture being gas A.

16. The composition of claim 15, wherein gas (B) is a fluorine-containing biocompatible gas.

17. The composition of claim 15, wherein the fluorine-containing gas is selected from the group consisting of SF₆, CF₄, C₂F₆, C₃F₆, C₃F₈, C₄F₆, C₄F₈, C₄F₁₀, C₅F₁₀, C₅F₁₂ and mixtures thereof.

18. The composition of claim 15, wherein gas A is selected from the group consisting of air, oxygen, nitrogen, carbon dioxide or mixtures thereof.

19. A dry pulverulent formulation which, upon dissolution in water, will form an aqueous suspension of stabilized air or gas microbubbles useful in ultrasonic imaging, the formulation comprising at least one film forming surfactant and at least one hydrophilic stabilizer in the presence of air or other entrappable gas, the film forming surfactant being a saturated phospholipid in lamellar or laminar form, wherein the surfactant comprises, bound thereto, bioactive species designed for specific targeting purposes.

20. The formulation of claim 19, in which the phosophilipid(s) are selected from the group consisting of phosphatidic acid, phosphatidylcholine, phosphatidylethanolamine, phosphatidylserine, phosphatidylglycerol, phosphatidylinositol, cardiolipin and sphingomyelin.

21. A method of imaging organs in a living body, said method comprising
administering to said body a composition consisting of a suspension of air or gas microbubbles in a physiologically acceptable aqueous carrier phase the suspension comprising one or more dissolved or dispersed surfactants, at least one of which is a film forming surfactant present in the composition at least partially in lamellar or laminar form; at least one of said surfactants comprising, bound thereto, bioactive species designed for specific targeting purposes and
subjecting said body to ultrasonic echography.

22. A method of imaging organs in a living body, said method comprising
administering to said body a composition consisting of a suspension of air or gas microbubbles in a physiologically acceptable aqueous carrier phase the suspension comprising one or more dissolved or dispersed surfactants, at least one of which is a film forming surfactant present in the composition at least partially in lamellar or laminar form, said surfactants forming a surfactant layer stabilizing said microbubbles, said surfactant layer having bound thereto monoclonal antibodies tailored by genetic engineering, antibody fragments or polypeptides designed to mimic antibodies, bioadhesive polymers, lectins or other site-recognizing molecules.; and
subjecting said body to ultrasonic echography.

23. The method of any one of claims 21 or 22 wherein the suspension contains at least 107 microbubbles per milliliter and wherein the concentration of the phospholipid(s) in the liquid carrier is below 0.01% by weight.

24. The method of claim 21, in which the concentration of microbubbles per milliliter is between 10⁸ and 10¹⁰.

25. The method of claim 23, in which the concentration of phospholipids is above 0.00013% wt.

26. The method of claim 21, in which the liquid carrier further comprises a stabilizer selected from the group consisting of water soluble poly- and oligosaccharides, sugars and hydrophilic polymers.

27. The method of claim 26, wherein a hydrophilic polymer is a polyethylene glycol.

28. The method of claim 21, in which the phosophilipid(s) are selected from the group consisting of lecithins such as phosphatidic acid, phosphatidylcholine, phosphatidylethanolamine, phosphatidylserine, phosphatidylglycerol, phosphatidylinositol, cardiolipin and sphingomyelin.

29. The method of claim 23, in which the suspension contains about 10⁸-10⁹ microbubbles per milliliter with the microbubble size between 0.5-10 mm showing little or no variation under storage.

30. The method of claim 21, in which the liquid carrier further comprises up to 50% by weight non-laminar surfactants selected from the group consisting of fatty acids, esters and ethers of fatty acids and alcohols with polyols such as polyalkalene glycols, polyalkylenated sugars and other carbohydrates, and polyalkylenated glycerol.

31. The method of claim 23, in which the microbubbles are filled with SF₆, CF₄, freons or air.

32. The method of claim 21 wherein microbubbles are filled with a freon gas selected from CF₄, CBrF₃, C₄F₈, CClF₃, CCl₂ F₂, C₂F₆, C₂ClF₅, CBrClF₂, C₂Cl₂F₄, CBr₂F₂ and C₄ F₁₀.

33. The method of claim 23 wherein the microbubbles are filled with a gas mixture of at least two biocompatible gases A and B in which at least one gas (B) present in an amount of between 0.5-41% by vol. has a molecular weight greater than 80 daltons and solubility in water below 0.0283 ml per ml of water at standard conditions, the balance of the mixture being gas A.

34. The method of claim 33, wherein gas (B) is a fluorine-containing biocompatible gas.

35. The method of claim 33, wherein the fluorine-containing gas is selected from the group consisting of SF₆, CF₄, C₂F₆, C₃F₆, C₃F₈, C₄F₆, C₄F₈, C₄F₁₀, C₅F₁₀, C₅F₁₂ and mixtures thereof.

36. The method of claim 33, wherein gas A is selected from the group consisting of air, oxygen, nitrogen, carbon dioxide or mixtures thereof.

37. A method for the preparation of a composition suitable for injection into the bloodstream and body cavities of living beings, comprising a suspension of stabilized air or gas microbubbles in a physiologically acceptable aqueous carrier phase having one or more dissolved or dispersed surfactants, at least one of said surfactants being a film forming saturated phospholipid present in the composition at least partially in lamellar or laminar form. wherein at least one of said surfactants comprise, bound thereto, bioactive species designed for specific targeting purposes., said method comprising the steps of:
(a) selecting at least one film forming surfactant and at least a surfactant comprising, bound thereto, bioactive species designed for specific targeting purposes and converting them into lamellar form;
(b) contacting the surfactants in lamellar form with air or an adsorbable or entrappable gas for a time sufficient for that air or gas to become bound by said surfactant; and
(c) admixing the surfactants in lamellar form with an aqueous liquid carrier, to form a stable dispersion of air or gas microbubbles in said liquid carrier.

38. The method of claim 37, wherein step (c) is performed before step (b), step (b) being effected by introducing pressurized air or gas into the liquid carrier and thereafter releasing the pressure.

39. The method of claim 37, wherein step (c) is brought about by gentle mixing of the components with no shaking, whereby air or gas bound to the lamellar surfactant in step (b) develops into a suspension of stable microbubbles.

40. The method of claim 37 wherein the suspension contains at least 10⁷ microbubbles per milliliter and wherein the concentration of the phospholipid(s) in the liquid carrier is below 0.01% by weight.

41. The method of claim 40, in which the concentration of microbubbles per milliliter is between 10⁸ and 10¹⁰.

42. The method of claim 40, in which the concentration of phospholipids is above 0.00013% wt.

43. The method of claim 37 , in which the liquid carrier further comprises a stabilizer selected from the group consisting of water soluble poly- and oligosaccharides, sugars and hydrophilic polymers.

44. The method of claim 43, wherein a hydrophilic polymer is a polyethylene glycol.

45. The method of claim 37, in which the phosophilipid(s) are selected from the group consisting of lecithins such as phosphatidic acid, phosphatidylcholine, phosphatidylethanolamine, phosphatidylserine, phosphatidylglycerol, phosphatidylinositol, cardiolipin and sphingomyelin.

46. The method of claim 40, in which the suspension contains about 10⁸-10⁹ microbubbles per milliliter with the microbubble size between 0.5-10 mm showing little or no variation under storage.

47. The method of claim 37, in which the liquid carrier further comprises up to 50% by weight non-laminar surfactants selected from the group consisting of fatty acids, esters and ethers of fatty acids and alcohols with polyols such as polyalkalene glycols, polyalkylenated sugars and other carbohydrates, and polyalkylenated glycerol.

48. The method of claim 37, in which the microbubbles are filled with SF₆, CF₄, freons or air.

49. The method of claim 37 wherein microbubbles are filled with a freon gas selected from CF₄, CBrF₃, C₄F₈, CClF₃, CCl₂ F₂, C₂F₆, C₂ClF₅, CBrClF₂, C₂Cl₂F₄, CBr₂F₂ and C₄ F₁₀.

50. The method of claim 37 wherein the microbubbles are filled with a gas mixture of at least two biocompatible gases A and B in which at least one gas (B) present in an amount of between 0.5-41% by vol. has a molecular weight greater than 80 daltons and solubility in water below 0.0283 ml per ml of water at standard conditions, the balance of the mixture being gas A.

51. The method of claim 50, wherein gas (B) is a fluorine-containing biocompatible gas.

52. The method of claim 50, wherein the fluorine-containing gas is selected from the group consisting of SF₆, CF₄, C₂F₆, C₃F₆, C₃F₈, C₄F₆, C₄F₈, C₄F₁₀, C₅F₁₀, C₅F₁₂ and mixtures thereof.

53. The method of claim 50, wherein gas A is selected from the group consisting of air, oxygen, nitrogen, carbon dioxide or mixtures thereof.

54. An ultrasound contrast agent comprising an aqueous suspension of gas filled microbubbles comprising a saturated phospholipid, a fatty acid, a hydrophilic stabilizer, and SF₆, wherein the amount of the saturated phospholipid in the suspension is less than about 0.01% by weight.

55. The ultrasound contrast agent of claim 54, wherein the fatty acid is present in an amount between 1% and 50% by weight of the amount of the saturated phospholipid.

56. The ultrasound contrast agent of claim 54, wherein the fatty acid is present in an amount between 10% and 15% by weight of the amount of the saturated phospholipid.

57. The ultrasound contrast agent of claim 54, wherein the fatty acid is a C₁₂-C.₂₄ straight chain saturated fatty acid selected from the group consisting of lauric acid, myristic acid, palmitic acid, stearic acid, arachidic acid, behenic acid, lignoceric acid and mixtures thereof.

58. The ultrasound contrast agent of claim 54, wherein the fatty acid comprises palmitic acid in an amount between 10% and 15% by weight of the amount of the saturated phospholipid.

59. The ultrasound contrast agent of claim 54, wherein the saturated phospholipid is selected from the group consisting of dimyristoylphosphatidic acid, dimyristoylphosphatidylglycerol, dimyristoylphosphatidylserine, dipalmitoylphosphatidic acid, dipalmitoylphosphatidylglycerol, dipalmitoylphosphatidylserine, distearoylphosphatidic acid, distearoylphosphatidylglycerol, distearoylphosphatidylserine and mixtures thereof.

60. The ultrasound contrast agent of claim 54, wherein the saturated phospholipid comprises distearoylphosphatidylcholine (DSPC) and dipalmitoylphosphatidylglycerol (DPPG).

61. A method of imaging a region of a body comprising: (a) administering to the body an aqueous suspension of gas filled microbubbles comprising a saturated phospholipid, a fatty acid, a hydrophilic stabilizer, and SF₆, wherein the amount of the saturated phospholipid in the suspension is less than 0.01% by weight; and (b) imaging the body.

62. The method of imaging of claim 61, wherein the fatty acid is present in an amount between 1% and 50% by weight of the amount of the saturated phospholipid.

63. The method of imaging of claim 61, wherein the fatty acid is present in an amount between 10% and 15% by weight of the amount of the saturated phospholipid.

64. The method of imaging of claim 61, wherein the fatty acid is a C₁₂-C₂₄ straight chain saturated fatty acid selected from the group consisting of lauric acid, myristic acid, palmitic acid, stearic acid, arachidic acid, behenic acid, lignoceric acid and mixtures thereof.

65. The method of imaging of claim 61, wherein the fatty acid comprises palmitic acid in an amount between 10% and 15% by weight of the amount of the saturated phospholipid.

66. The method of imaging of claim 61, wherein the saturated phospholipid is selected from the group consisting of dimyristoylphosphatidic acid, dimyristoylphosphatidylglycerol, dimyristoylphosphatidylserine, dipalmitoylphosphatidic acid, dipalmitoylphosphatidylglycerol, dipalmitoylphosphatidylserine, distearoylphosphatidic acid, distearoylphosphatidylglycerol, distearoylphosphatidylserine and mixtures thereof.

67. The method of imaging of claim 61, wherein the saturated phospholipid comprises distearoylphosphatidylcholine (DSPC) and dipalmitoylphosphatidylglycerol (DPPG).

68. The method of imaging of claim 61, wherein the hydrophilic stabilizer comprises PEG 4000.

69. The method of imaging of claim 61, wherein the saturated phospholipid comprises distearoylphosphatidylcholine (DSPC) and dipalmitoylphosphatidylglycerol (DPPG), the fatty acid comprises palmitic acid in an amount between 10% and 15% by weight of the amount of the saturated phospholipid, and the hydrophilic stabilizer comprises PEG 4000.

70. The method of imaging of claim 61, wherein the body is a vertebrate and the suspension is administered to the vasculature or body cavity of the vertebrate.

71. A dry formulation of an ultrasound contrast agent comprising a saturated phospholipid, a fatty acid, and a hydrophilic stabilizer, wherein upon dissolution in an aqueous carrier liquid, the dry formulation will form a suspension of microbubbles comprising SF₆ in which the amount of saturated phospholipid in the suspension is less than about 0.01% by weight.

72. The dry formulation of claim 71, wherein the fatty acid is present in an amount of between 1% and 50% by weight of the amount of the saturated phospholipid.

73. The dry formulation of claim 71, wherein the fatty acid is present in an amount of between 5% and 25% by weight of the amount of the saturated phospholipid.

74. The dry formulation of claim 71, wherein the fatty acid is present in an amount of between 10% and 15% by weight of the amount of the saturated phospholipid.

75. The dry formulation of claim 71, wherein the fatty acid is a C₁₂-C₂₄ straight chain saturated fatty acid selected from the group consisting of lauric acid, myristic acid, palmitic acid, stearic acid, arachidic acid, behenic acid, lignoceric acid and mixtures thereof.

76. The dry formulation of claim 71, wherein the fatty acid comprises palmitic acid in an amount of between 10% and 15% by weight of the amount of the saturated phospholipid.

77. The dry formulation of claim 71, wherein the saturated phospholipid is selected from the group consisting of dimyristoylphosphatidic acid, dimyristoylphosphatidylglycerol, dimyristoylphosphatidylserine, dipalmitoylphosphatidic acid, dipalmitoylphosphatidylglycerol, dipalmitoylphosphatidylserine, distearoylphosphatidic acid, distearoylphosphatidylglycerol, distearoylphosphatidylserine and mixtures thereof.

78. The dry formulation of claim 71, wherein the saturated phospholipid comprises distearoylphosphatidylcholine (DSPC) and dipalmitoylphosphatidylglycerol (DPPG).

79. The dry formulation of any one of claims 71, 76 or 78 wherein the hydrophilic stabilizer comprises PEG 4000.

80. The dry formulation of claim 71, wherein the saturated phospholipid comprises distearoylphosphatidylcholine (DSPC) and dipalmitoylphosphatidylglycerol (DPPG), the fatty acid comprises palmitic acid in an amount of between 10% and 15% by weight of the amount of the saturated phospholipid, and the hydrophilic stabilizer comprises PEG 4000.

81. A method of preparing an ultrasound contrast agent comprising reconstituting a dry formulation of an ultrasound contrast agent comprising a saturated phospholipid, a fatty acid, and a hydrophilic stabilizer, in an aqueous carrier liquid to form a suspension of microbubbles comprising SF₆ in which the amount of saturated phospholipid in the suspension is less than about 0.01% by weight.

82. The method of claim 81, wherein the fatty acid is present in an amount of between 1% and 50% by weight of the amount of the saturated phospholipid.

83. The method of claim 81, wherein the fatty acid is present in an amount of between 5% and 25% by weight of the amount of the saturated phospholipid.

84. The method of claim 81, wherein the fatty acid is present in an amount of between 10% and 15% by weight of the amount of the saturated phospholipid.

85. The method of claim 81, wherein the fatty acid is a C₁₂-C₂₄ straight chain saturated fatty acid selected from the group consisting of lauric acid, myristic acid, palmitic acid, stearic acid, arachidic acid, behenic acid, lignoceric acid and mixtures thereof.

86. The method of claim 81, wherein the fatty acid comprises palmitic acid in an amount of between 10% and 15% by weight of the amount of the saturated phospholipid.

87. The method of claim 81, wherein the saturated phospholipid is selected from the group consisting of dimyristoylphosphatidic acid, dimyristoylphosphatidylglycerol, dimyristoylphosphatidylserine, dipalmitoylphosphatidic acid, dipalmitoylphosphatidylglycerol, dipalmitoylphosphatidylserine, distearoylphosphatidic acid, distearoylphosphatidylglycerol, distearoylphosphatidylserine and mixtures thereof.

88. The method of claim 81, wherein the saturated phospholipid comprises distearoylphosphatidylcholine (DSPC) and dipalmitoylphosphatidylglycerol (DPPG).

89. The method of claim 81, wherein the hydrophilic stabilizer comprises PEG 4000.

90. The method of claim 81, wherein the saturated phospholipid comprises distearoylphosphatidylcholine (DSPC) and dipalmitoylphosphatidylglycerol (DPPG), the fatty acid comprises palmitic acid in an amount of between 10% and 15% by weight of the amount of the saturated phospholipid, and the hydrophilic stabilizer comprises PEG 4000.

91. A method of imaging a region of a body comprising: (a) reconstituting a dry formulation of an ultrasound contrast agent comprising a saturated phospholipid, a fatty acid, and a hydrophilic stabilizer, in an aqueous carrier liquid to form a suspension of microbubbles comprising SF₆ in which the amount of saturated phospholipid in the suspension is less than about 0.01% by weight; (b) administering the suspension of gas filled microbubbles to the body; and (c) imaging the body.

92. The method of claim 91, wherein the fatty acid is present in an amount of between 1% and 50% by weight of the amount of the saturated phospholipid.

93. The method of claim 91, wherein the fatty acid is present in an amount of between 5% and 25% by weight of the amount of the saturated phospholipid.

94. The method of claim 91, wherein the fatty acid is present in an amount of between 10% and 15% by weight of the amount of the saturated phospholipid.

95. The method of claim 91, wherein the fatty acid is a C₁₂-C₂₄ straight chain saturated fatty acid selected from the group consisting of lauric acid, myristic acid, palmitic acid, stearic acid, arachidic acid, behenic acid, lignoceric acid and mixtures thereof.

96. The method of claim 91, wherein the fatty acid comprises palmitic acid in an amount of between 10% and 15% by weight of the amount of the saturated phospholipid.

97. The method of claim 91, wherein the saturated phospholipid is selected from the group consisting of dimyristoylphosphatidic acid, dimyristoylphosphatidylglycerol, dimyristoylphosphatidylserine, dipalmitoylphosphatidic acid, dipalmitoylphosphatidylglycerol, dipalmitoylphosphatidylserine, distearoylphosphatidic acid, distearoylphosphatidylglycerol, distearoylphosphatidylserine and mixtures thereof.

98. The method of claim 91, wherein the saturated phospholipid comprises distearoylphosphatidylcholine (DSPC) and dipalmitoylphosphatidylglycerol (DPPG).

99. The method of claim 91, wherein the hydrophilic stabilizer comprises PEG 4000.

100. The method of claim 91, wherein the saturated phospholipid comprises distearoylphosphatidylcholine (DSPC) and dipalmitoylphosphatidylglycerol (DPPG), the fatty acid comprises palmitic acid in an amount of between 10% and 15% by weight of the amount of the saturated phospholipid, and the hydrophilic stabilizer comprises PEG 4000.

101. A method of preparing an ultrasound contrast agent comprising an aqueous suspension of gas filled microbubbles comprising SF₆ and saturated phospholipid, wherein the amount of saturated phospholipid in the suspension is less than about 0.01% by weight, the method comprising: (a) dissolving at least one saturated phospholipid, a fatty acid, and a hydrophilic stabilizer in an organic solvent to form a solution; (b) freeze drying or spray drying the solution to form a dried powder; (c) contacting the dried powder with SF₆; and (d) mixing the freeze dried or spray dried powder with an aqueous carrier phase.

102. A method of preparing a dry formulation of an ultrasound contrast agent, wherein upon dissolution in an aqueous carrier liquid, the dry formulation will form a suspension of microbubbles comprising SF₆ and saturated phospholipid, wherein the amount of the saturated phospholipid in the suspension is less than about 0.01% by weight, the method comprising: (a) dissolving at least one saturated phospholipid, a fatty acid, and a hydrophilic stabilizer in an organic solvent to form a solution; (b) freeze drying or spray drying the solution to form a dried powder; and (c) contacting the dried powder with SF₆.

103. A method of preparing a dry formulation of an ultrasound contrast agent, wherein upon dissolution in an aqueous carrier liquid, the dry formulation will form a suspension of microbubbles comprising SF₆ and saturated phospholipid, wherein the amount of the saturated phospholipid in the suspension is less than about 0.01% by weight, the method comprising: (a) dissolving at least one saturated phospholipid and a hydrophilic stabilizer in an organic solvent to form a solution; (b) freeze drying or spray drying the solution to form a dried powder; (c) mixing the dried powder with a fatty acid to form a mixture; and (d) contacting the mixture with SF₆.
